(19) United States
(12) Patent Application Publication
(10) Pub. No.: US 2026/0306770 A1
(43) Pub. Date: Oct. 8, 2026

(54) DEVICE FOR SUPPLYING BREATING GAS

(71) Applicant: LÖEWENSTEIN MEDICAL TECHNOLOGY S.A., Luxembourg (LU)

(72) Inventors: Peter KREMEIER, Karlsruhe (DE); Lorenz LENHARDT, Griesheim (DE); Andreas SCHOBER, Frankfurt am Main (DE)

(21) Appl. No.: 19/476,963

(22) PCT Filed: Apr. 17, 2024
(86) PCT No.: PCT/EP2024/060450

§ 371 Date: Oct. 20, 2025

(30) Foreign Application Priority Data
Apr. 25, 2023 (DE) 10 2023 110 586.8
Jun. 12, 2023 (LU) 103147

(51) Int. Cl. A61M 16/01; A61M 16/00; A61M 16/08; A61M 16/10; A61M 16/20

(52) U.S. Cl. CPC A61M 16/01; A61M 16/0003; A61M 16/0051; A61M 16/0093; A61M 16/0891; A61M 16/10; A61M 16/204; A61M 16/205; A61M 16/208; A61M 2202/0007; A61M 2202/0014; A61M 2202/0208; A61M 2202/0225; A61M 2202/0241; A61M 2205/18; A61M 2205/3327; A61M 2205/3331; A61M 2205/50; A61M 2205/75; A61M 2205/8206

(57) ABSTRACT

The invention relates to a breathing gas supply device having at least one breathing gas line for conducting a breathing gas mixture, wherein the breathing gas line comprises at least one outlet via which the breathing gas mixture can be at least partially, at least temporarily, discharged, wherein the breathing gas line comprises an inspiratory branch which is designed to conduct breathing gas to a connection for a patient interface, wherein the breathing gas line comprises an expiratory branch which is designed to conduct breathing gas between the connection for a patient interface and the outlet, characterised in that at least one shut-off valve is arranged in the breathing gas line, which is designed and configured to at least periodically establish a breathing-gas-conducting connection from the expiratory branch to the inspiratory branch.

The invention relates to a device for anesthesia and/or ventilation.

Anesthesia workstations are typically constructed so that the breathing gases are guided in a circuit and consumed gases such as oxygen (O2) are supplied and carbon dioxide (CO2) is removed from the circuit. Volatile anesthetic agents can be added and separated in a controlled manner in such a circuit. The gases flow in a defined direction due to a breathing gas source and check valves. The inspiratory air is guided via the inspiratory branch of the breathing gas circuit to the patient. After the inspiration, the expiration air of the patient reaches the circulation system again via the expiratory branch. CO2 is then separated from the expiration air, the breathing gas is mixed with new fresh gases and supplied to the patient again. The CO2 is typically withdrawn from the breathing gas mixture by a chemical CO2 absorber.

It is the object of the present invention to provide a device which can be used both for applying volatile anesthetics and for ventilation or for respiratory support and is to be operated easily, flexibly, and nonetheless safely.

This object is achieved by a device of claim 1. Refinements and advantageous embodiments are the subject matter of the dependent claims. Further advantages and features result from the general description and the description of the exemplary embodiments.

It is to be noted that the features set forth individually in the claims can be combined with one another in any technically reasonable manner and disclose further embodiments of the invention. The description additionally characterizes and specifies the invention in particular in conjunction with the figures. The dependent claims relate to various advantageous refinements of the present invention, which are independent of one another and the features of which can be combined freely with one another by a person skilled in the art in the scope of what is technically reasonable. This also applies across the boundaries of the various claim categories.

The invention relates to a device for supplying breathing gas having at least one breathing gas line for conducting a breathing gas mixture, wherein the breathing gas line comprises at least one outlet, via which the breathing gas mixture can be at least partially, at least temporarily discharged, wherein the breathing gas line comprises an inspiratory branch, which is designed to conduct breathing gas to a fitting for a patient interface, wherein the breathing gas line comprises an expiratory branch, which is designed to conduct breathing gas between the fitting for a patient interface and the outlet, characterized in that at least one shutoff valve is arranged in the breathing gas line, which is designed and configured to at least temporarily establish a connection conducting breathing gas from the expiratory branch to the inspiratory branch.

In some embodiments, the device is characterized in that the device comprises at least one reservoir for the breathing gas mixture and at least one fan, which is configured to provide a conveying energy for the breathing gas mixture, wherein reservoir and fan are arranged in or at the inspiratory branch.

In some embodiments, the device is characterized in that the shutoff valve is designed as a check valve and is configured to establish the connection conducting breathing gas in a flow direction from the expiratory branch to the inspiratory branch.

In some embodiments, the device is characterized in that the shutoff valve is designed as a blockable check valve and is configured to at least temporarily block the breathing gas line in both flow directions.

In some embodiments, the device is characterized in that the device comprises a control unit and at least one power source. In some embodiments, the device is characterized in that the shutoff valve is configured, when energized by the at least one power source, to block the breathing gas line in both flow directions. In some embodiments, the device is characterized in that the shutoff valve is configured, when energized, to suppress the connection conducting breathing gas from the expiratory branch to the inspiratory branch. In some embodiments, the device is characterized in that the breathing gas mixture, when the shutoff valve is energized, can be discharged completely from the expiratory branch via the outlet.

In some embodiments, the device is characterized in that inspiratory branch and expiratory branch, without the shutoff valve being energized, form at least one first circuit in which the breathing gas mixture can be conducted, wherein the breathing gas mixture can be at least partially discharged via the outlet.

In some embodiments, the device is characterized in that the device comprises at least one variable pressure control valve. In some embodiments, the device is characterized in that the pressure control valve is configured to control an inspiration pressure Pinsp and/or an expiration pressure Pexsp. In some embodiments, the device is characterized in that the pressure control valve is manually and/or electrically settable.

In some embodiments, the device is characterized in that the device comprises an APL valve for controlling the inspiration pressure Pinsp. In some embodiments, the device is characterized in that the breathing gas mixture can be discharged via the outlet when the pressure in the breathing gas line exceeds the inspiratory pressure Pinsp. In some embodiments, the device is characterized in that the APL valve is configured as a variably loaded check valve. In some embodiments, the device is characterized in that the APL valve comprises a stepping motor, via which the APL valve is settable. In some embodiments, the device is characterized in that the valve position of the APL valve is set and/or maintained when energized. In some embodiments, the device is characterized in that the valve position of the APL valve remains at the last set value without energizing. In some embodiments, the device is characterized in that the APL valve is manually and/or electrically settable.

In some embodiments, the device is characterized in that the device comprises a pressure control valve for controlling the expiration pressure Pexsp. In some embodiments, the device is characterized in that the pressure control valve is arranged in the expiratory branch. In some embodiments, the device is characterized in that the pressure control valve is configured to control an end-expiratory expiration pressure PEEP. In some embodiments, the device is characterized in that the pressure control valve is configured, without being energized, to passively regulate to a preset expiration pressure Pexsp. In some embodiments, the device is characterized in that the preset expiration pressure Pexsp of the pressure control valve is 3 hPa to 10 hPa, for example 5 hPa.

In some embodiments, the device is characterized in that the breathing gas mixture contains fresh gas and/or oxygen O2 and/or volatile anesthetics. In some embodiments, the device is characterized in that the device comprises at least one anesthetic supply line for introducing volatile anesthetics into the breathing gas line.

In some embodiments, the device is characterized in that a first pressure is applied in the breathing gas line and a second pressure is applied in the anesthetic supply line, wherein the first pressure is less than the second pressure. In some embodiments, the device is characterized in that the second pressure is at least 100 kPa, preferably at least 180 kPa. In some embodiments, the device is characterized in that the first pressure is less than 100 kPa, preferably less than 50 kPa, particularly preferably less than 10 kPa. In some embodiments, the device is characterized in that the first pressure is less than 8 kPa, preferably less than 5 kPa, particularly preferably less than 3 kPa. In some embodiments, the device is characterized in that the device is operable at room temperature.

In some embodiments, the device is characterized in that the volatile anesthetics are selected from the following group: isoflurane, sevoflurane, desflurane, halothane, enflurane, methoxyflurane. In some embodiments, the device is characterized in that the volatile anesthetics can be conducted in the anesthetic supply line in liquid form to the breathing gas line. In some embodiments, the device is characterized in that the volatile anesthetics vaporize upon introduction into the breathing gas line at a vaporization rate of 0 to 2 l/min. In some embodiments, the device is characterized in that the vaporized volatile anesthetics mix in the breathing gas line with the breathing gas mixture.

In some embodiments, the device is characterized in that the device comprises at least one safety valve which is configured and designed to block the supply of volatile anesthetics. In some embodiments, the device is characterized in that the safety valve is designed as a switching valve. In some embodiments, the device is characterized in that the safety valve is electrically and/or manually settable. In some embodiments, the device is characterized in that the safety valve is designed as an electrically operated switching valve, wherein the safety valve is configured without energizing to block the introduction of volatile anesthetics into the breathing gas mixture.

In some embodiments, the device is characterized in that the outlet comprises at least one filter. In some embodiments, the device is characterized in that the filter is exchangeable. In some embodiments, the device is characterized in that breathing gas mixture discharged via the outlet completely passes the filter. In some embodiments, the device is characterized in that the filter is an absorbent and/or comprises an absorbent and is configured and designed to absorb at least volatile anesthetics and/or the metabolites thereof. In some embodiments, the device is characterized in that the filter comprises activated carbon. In some embodiments, the device is characterized in that a double filter system having a first filter and a second filter is arranged at the outlet, wherein the second filter is arranged after the first filter in the flow direction.

In some embodiments, the device is characterized in that the device comprises at least one sensor which is arranged in or at the outlet. In some embodiments, the device is characterized in that the sensor is designed and configured to detect the concentration of volatile anesthetics and/or the metabolites thereof and transmit it to the control unit. In some embodiments, the device is characterized in that the concentration of volatile anesthetics and/or the metabolites thereof is detectable at least before and/or after the first filter in the flow direction and transmittable to the control unit.

In some embodiments, the device is characterized in that the control unit is configured to generate an alarm if the concentration of volatile anesthetics and/or the metabolites thereof exceeds a limiting value.

In some embodiments, the device is characterized in that the device comprises a separating means which is configured to separate at least CO2 from the breathing gas mixture. In some embodiments, the device is characterized in that the separating means is a chemical separating means and/or a mechanical separating means.

In some embodiments, the device is characterized in that the chemical separating means contains at least one CO2-binding absorbent, which is selected from the following group: calcium hydroxide, sodium hydroxide, potassium hydroxide, barium hydroxide, soda lime. In some embodiments, the device is characterized in that expiratory breathing gas exsp can flow through the chemical separating means if the inspiratory branch and the expiratory branch form the circuit.

In some embodiments, the device is characterized in that the mechanical separating means is arranged in the expiratory branch. In some embodiments, the device is characterized in that the mechanical separating means is adjacent to the fitting for a patient interface. In some embodiments, the device is characterized in that the separating means comprises at least one diffusion filter, which is designed as a semipermeable membrane and is permeable at least to CO2 molecules. In some embodiments, the device is characterized in that the diffusion filter is not permeable at least for volatile anesthetics.

In some embodiments, the device is characterized in that the separating means is designed as a two-chamber system, which comprises at least one first chamber and at least one second chamber, wherein the first chamber and the second chamber are gas conducting and are separated from one another by the diffusion filter. In some embodiments, the device is characterized in that the first chamber is configured to receive expiratory breathing gas exsp and the second chamber is configured to receive a sweep gas, wherein the sweep gas at least has a lower CO2 concentration than the expiratory breathing gas exsp. In some embodiments, the device is characterized in that the first chamber is configured to guide the expiratory breathing gas exsp in the direction of a main flow, wherein the second chamber is configured to guide the sweep gas in the direction of a sweep gas flow, wherein the flow direction of the main flow is opposite to the flow direction of the sweep gas flow. In some embodiments, the device is characterized in that the CO2 concentration of the sweep gas upon introduction into the second chamber is less than 10%, preferably less than 5%, particularly preferably is 0%.

In some embodiments, the device is characterized in that the device comprises at least one sweep gas supply line for providing sweep gas for the mechanical separating means, wherein the sweep gas supply line comprises at least one valve for metering sweep gas. In some embodiments, the device is characterized in that the flow rate of the sweep gas is greater than or equal to the flow rate of the expiratory breathing gas exsp. In some embodiments, the device is characterized in that the flow rate of the sweep gas is 0 to 20 l/min, preferably 0 to 10 l/min. In some embodiments, the device is characterized in that the flow rate of the sweep gas is set in relation to the minute volume. In some embodiments, the device is characterized in that the flow rate of the sweep gas is 1.1 to 2 times the minute volume, preferably 1.2 to 1.5 times the minute volume.

In some embodiments, the device is characterized in that the device comprises at least one valve for metering fresh gas and/or oxygen O2. In some embodiments, the device is characterized in that the device comprises at least one valve for metering volatile anesthetics. In some embodiments, the device is characterized in that the metering valves are selected from the following group: needle valve, proportional valve, switching valve, aperture, throttle valve. In some embodiments, the device is characterized in that the metering valves are designed as needle valves. In some embodiments, the device is characterized in that the metering valves each comprise at least one stepping motor, via which the metering valves are settable. In some embodiments, the device is characterized in that the valve position of the metering valves is set and/or maintained when energized. In some embodiments, the device is characterized in that the valve position of the metering valves without energizing remains at the last set value and/or drops to an open base setting. In some embodiments, the device is characterized in that the device comprises at least one sensor for detecting at least one ventilation-specific parameter.

In some embodiments, the device is characterized in that the ventilation-specific parameters comprise at least one of the following parameters: inspiratory patient pressure, inspiratory patient flow, inspiratory tidal volume, inspiratory minute volume, inspiratory breathing rate, inspiratory O concentration, inspiratory CO concentration, inspiratory NO concentration, inspiratory anesthetic gas concentration; expiratory patient pressure, expiratory patient flow, expiratory tidal volume, expiratory minute volume, expiratory breathing rate, expiratory O concentration, expiratory CO concentration, expiratory NO concentration, expiratory anesthetic gas concentration; gas temperature, gas moisture, fresh gas flow, leakage.

In some embodiments, the device is characterized in that the device comprises at least one storage unit which is configured and designed to store at least the ventilation-specific parameters detected during the breathing gas supply.

In some embodiments, the device is characterized in that the storage unit is configured to store patient parameters, wherein the patient parameters comprise at least one of the following parameters: age, weight, height, body mass index (BMI), pre-existing conditions. In some embodiments, the device is characterized in that the flow rate of the sweep gas is dynamically adaptable to the ventilation-specific parameters and/or to the patient parameters.

In some embodiments, the device is characterized in that the breathing gas line comprises a bypass, which branches off from the inspiratory branch and opens into the expiratory branch between the mechanical separating means and the outlet. In some embodiments, the device is characterized in that the bypass is configured to at least temporarily establish a connection conducting breathing gas from the inspiratory branch to the expiratory branch such that a second circuit is formed in which the breathing gas mixture can be conducted. In some embodiments, the device is characterized in that the second circuit at least partially corresponds to the first circuit, wherein the fitting for a patient interface is only arranged in the first circuit.

In some embodiments, the device is characterized in that the mechanical separating means is only arranged in the first circuit.

In some embodiments, the device is characterized in that the bypass comprises at least one bypass shutoff valve. In some embodiments, the device is characterized in that the bypass shutoff valve is designed as a check valve and is configured to at least temporarily establish the connection conducting breathing gas in a flow direction from the inspiratory branch to the expiratory branch. In some embodiments, the device is characterized in that the bypass shutoff valve is designed as a blockable check valve and is configured to at least temporarily block the bypass in both flow directions. In some embodiments, the device is characterized in that the bypass shutoff valve is configured, when energized by the at least one power source, to block the bypass in both flow directions.

In some embodiments, the device is characterized in that the breathing gas mixture, without energizing of the shutoff valve and the bypass shutoff valve, can be conducted at least partially in the second circuit.

In some embodiments, the device is characterized in that the breathing gas mixture can be conducted in the first circuit with the main flow and the breathing gas mixture can be conducted in the second circuit with a bypass flow. In some embodiments, the device is characterized in that the main flow is dependent on the breathing phases of a patient to be ventilated. In some embodiments, the device is characterized in that the bypass flow is independent of the breathing phases of a patient to be ventilated.

In some embodiments, the device is characterized in that at least the bypass flow of the second circuit is conducted through the PEEP valve.

In some embodiments, the device is characterized in that the device is operable in different operating modes. In some embodiments, the device is characterized in that the device is operable in automatic operating modes, in which the fan supplies the conveying energy for the breathing gas mixture. In some embodiments, the device is characterized in that the device is operable in manual operating modes, in which the reservoir supplies the conveying energy for the breathing gas mixture. In some embodiments, the device is characterized in that the reservoir is designed as a manual bag.

In some embodiments, the device is characterized in that the device is operable in operating modes with application of volatile anesthetics and/or in operating modes without application of volatile anesthetics, wherein the safety valve is configured, when energized, to permit the introduction of volatile anesthetics into the breathing gas mixture and, without energizing, to block the introduction of volatile anesthetics into the breathing gas mixture.

In some embodiments, the device is characterized in that the device is operable in an operating mode which is selected from the following group: anesthesia mode with volatile anesthetics; anesthesia mode with intravenously administered anesthetics (TIVA mode); ventilation mode with anesthetics; ventilation mode without anesthetics, O therapy mode, high flow O therapy mode (HFOT mode), CPAP mode, bilevel mode, SIMV mode, emergency mode.

In some embodiments, the device is characterized in that the operating mode is manually and/or automatically settable by the control unit.

In some embodiments, the device is characterized in that the device is operable in an emergency mode without power supply and/or in a power saving manner. In some embodiments, the device is characterized in that the emergency mode is entered automatically and/or is manually settable. In some embodiments, the device is characterized in that the emergency mode is entered automatically when the functions of the power source and/or the control unit and/or the fan are restricted or fail.

In some embodiments, the device is characterized in that the breathing gas mixture can be conducted at least in the first circuit in the emergency mode, wherein the breathing gas mixture can be at least partially discharged via the outlet. In some embodiments, the device is characterized in that the breathing gas mixture can be conducted in the second circuit in the emergency mode. In some embodiments, the device is characterized in that the conveying energy for the breathing gas mixture is provided by the reservoir in the emergency mode.

In some embodiments, the device is characterized in that the APL valve regulates to the last set inspiratory pressure Pinsp in the emergency mode. In some embodiments, the device is characterized in that the pressure control valve passively regulates the expiratory pressure Pexsp in the emergency mode.

In some embodiments, the device is characterized in that the chemical separating means and/or the mechanical separating means is active without energizing.

In some embodiments, the device is characterized in that the safety valve is closed in the emergency mode, wherein the safety valve is manually openable.

In some embodiments, the device is characterized in that the metering valves are not energized in the emergency mode, so that the metering of fresh gas and/or oxygen and/or volatile anesthetics and/or sweep gas is kept at the last set value. In some embodiments, the device is characterized in that the valve for metering sweep gas sets the sweep gas metering to 1.2 to 2 times the last set minute volume in the emergency mode, preferably to 1.5 times. In some embodiments, the device is characterized in that fresh gas and/or oxygen are supplied via the metering valves to the breathing gas mixture in the emergency mode.

In some embodiments, the device is characterized in that the fresh gas and/or the oxygen are each provided in at least one pressurized gas cylinder, wherein the pressure of the pressurized gas cylinders supplies the conveying energy for the fresh gas and/or the oxygen.

In some embodiments, the device is characterized in that the volatile anesthetics are each provided in at least one tank, wherein a pressure is applied in the tanks. In some embodiments, the device is characterized in that the pressure in the tanks is at least 100 kPa, preferably at least 180 kPa. In some embodiments, the device is characterized in that the pressure in the tanks supplies the conveying energy for the volatile anesthetic.

In a further aspect, the invention relates to a method for introducing at least one volatile anesthetic into a breathing gas mixture, wherein the breathing gas mixture is conducted standing under a first pressure in a breathing gas line, characterized in that the volatile anesthetic is conducted in liquid form in an anesthetic supply line standing under a second pressure to the breathing gas line, wherein the first pressure is less than the second pressure, wherein the volatile anesthetic is subjected to the first pressure with entry into the breathing gas line and becomes gaseous.

The device 100 according to the invention is described by way of example in the following exemplary embodiments. Further features and advantages of the present invention will become clear in the following descriptions of exemplary embodiments on the basis of the figures. The invention is not restricted to the exemplary embodiments shown.

The device 100 is designed for breathing gas supply. The device 100 has a ventilation function and/or an anesthesia function. The device 100 can therefore be used as a ventilation device or as an anesthesia device. The device 100 can also be used as a ventilation device and as an anesthesia device. A patient can thus be ventilated or assisted in breathing and alternatively or additionally also kept under anesthesia by use of the device 100.

A ventilation device is to be understood to include all devices which assist a user or patient in the natural breathing and/or take over the ventilation of a user or patient and/or are used for respiratory therapy and/or act in another way on the breathing of a user or patient. The term “ventilation” used herein includes all forms of ventilation, breathing assistance, or respiratory therapy in the sense of the invention. Therefore, anesthesia sometimes also falls under the term “ventilation” herein, namely always for the case that volatile anesthetics or anesthetic gases are added to the breathing gas.

A user or patient can be connectable to the device 100 via a patient interface 91. A patient interface 91 is to be understood in the meaning of the invention as any peripheral device which is designed for interaction with an individual. The patient interface 91 can be designed as a tracheal tube or tracheal cannula. The patient interface 91 can also be designed as a breathing mask, nose mask, nose pad mask, nasal cannula or oxygen cannula, full face or total face mask. The patient interface 91 is preferably configured and designed such that a leak-free supply and discharge of a breathing gas mixture from the device 100 to the patient and/or from the patient to the device 100 can be carried out.

The figures show exemplary embodiments of the device according to the invention:

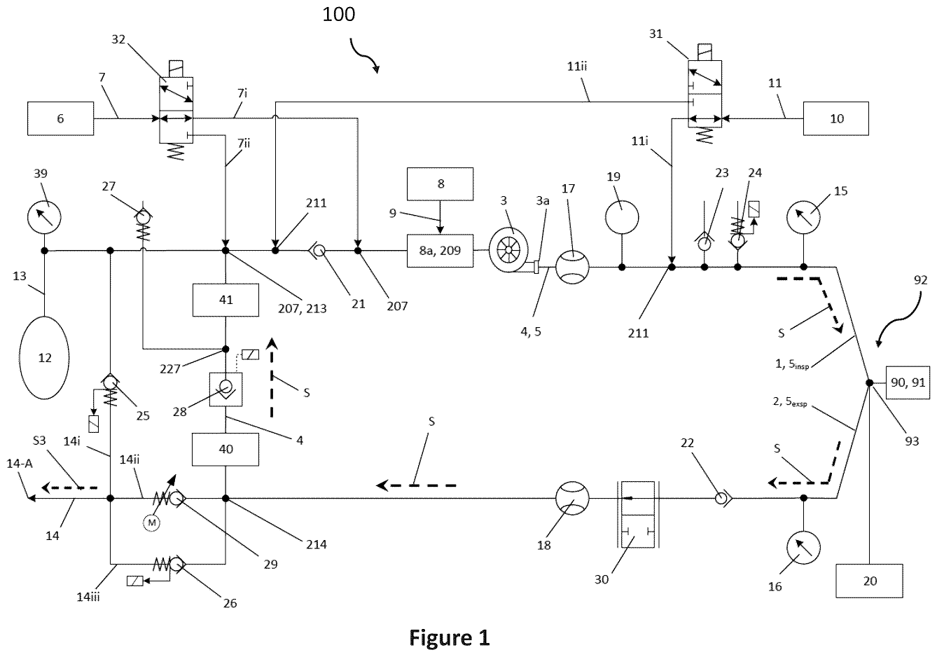

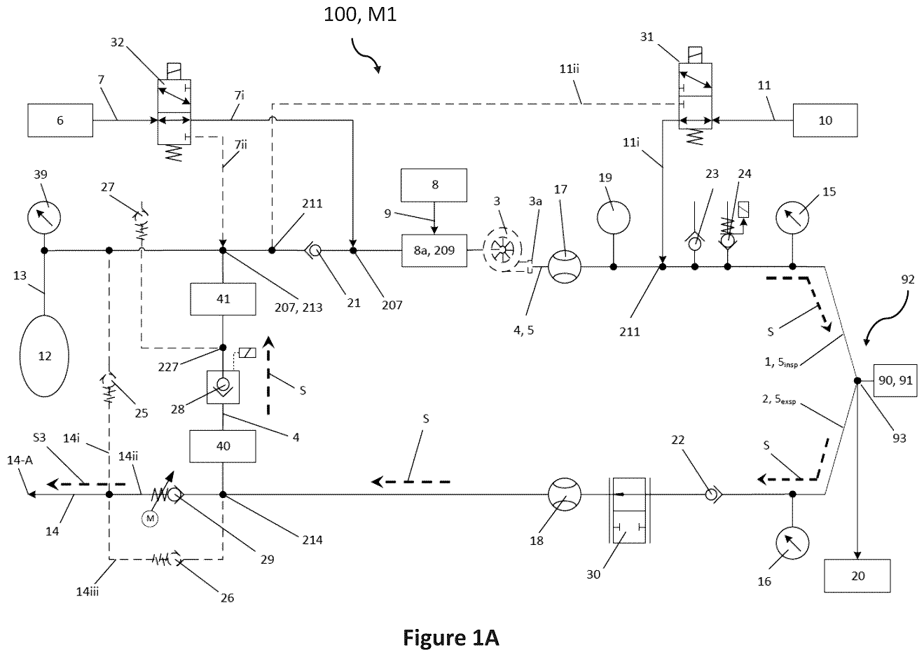

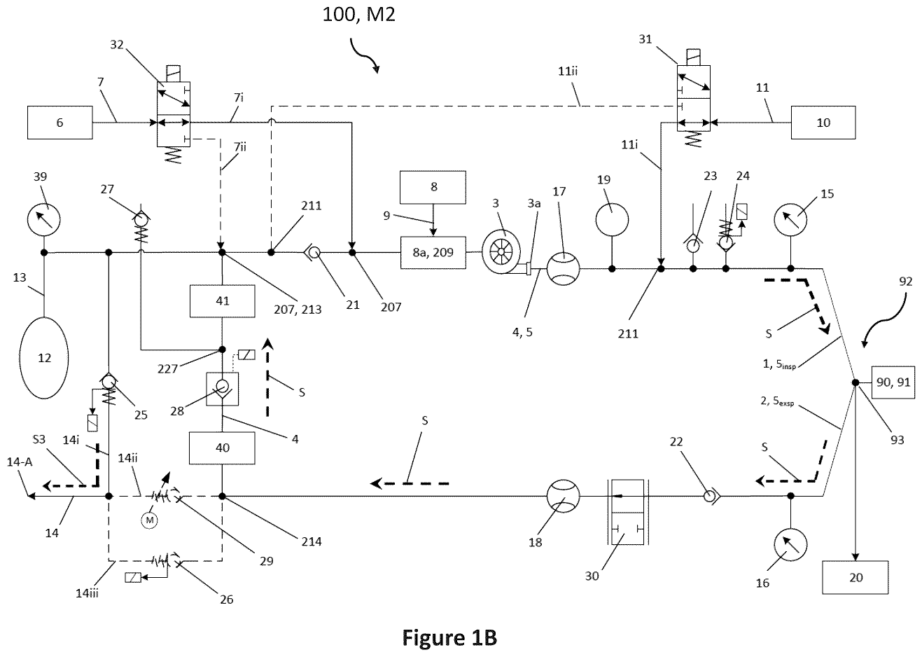

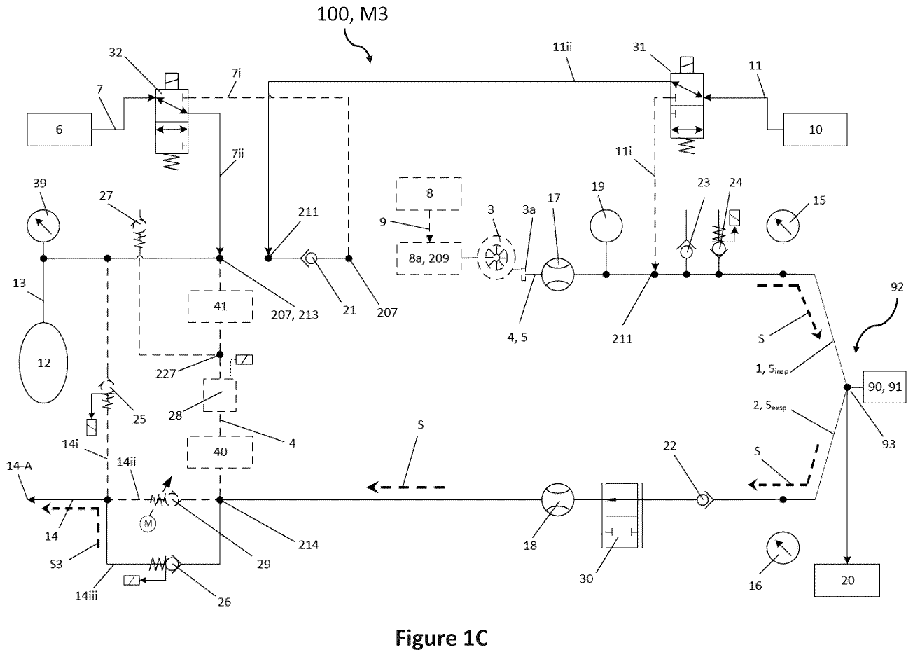

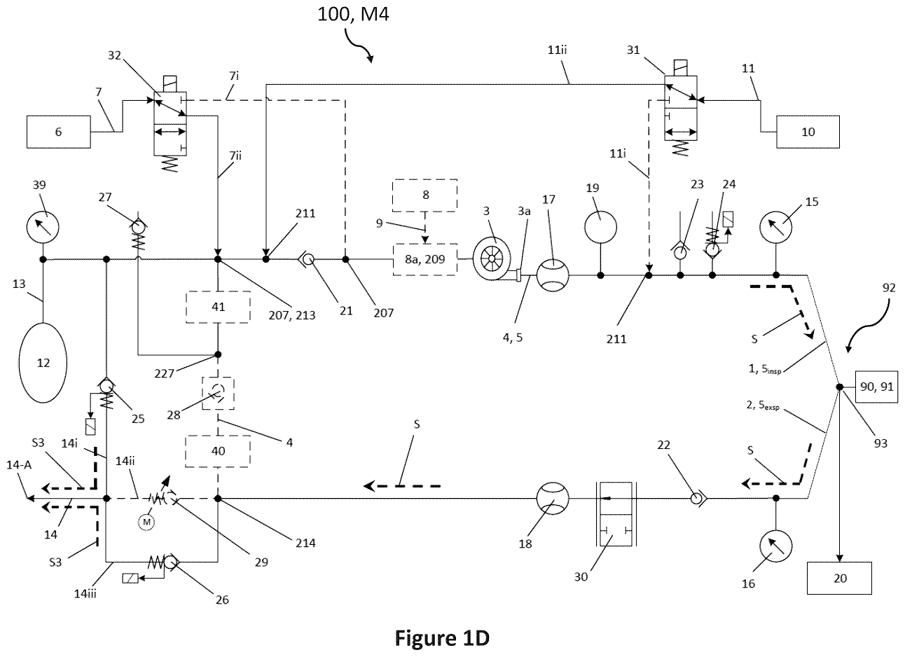

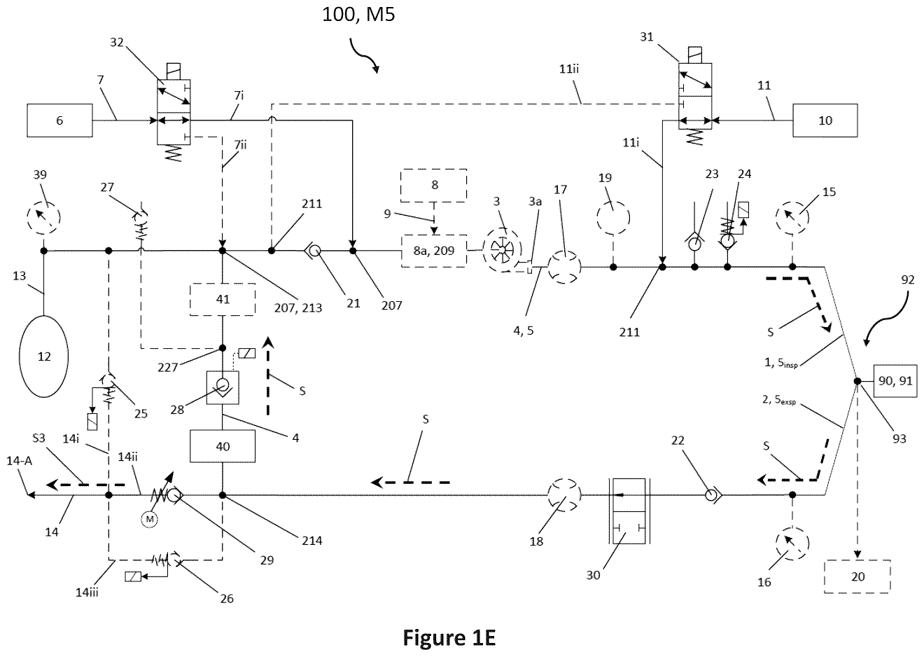

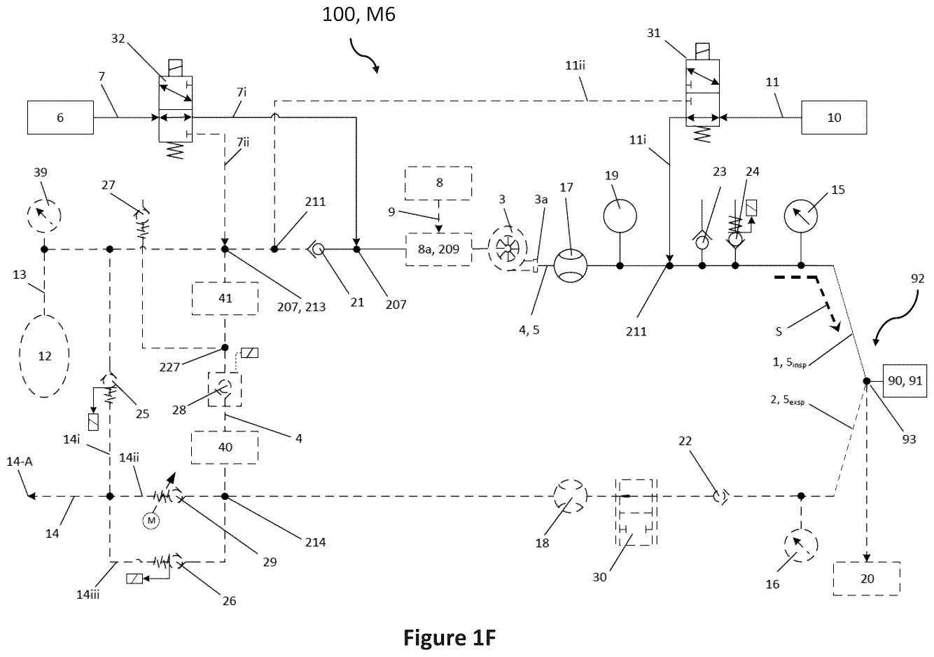

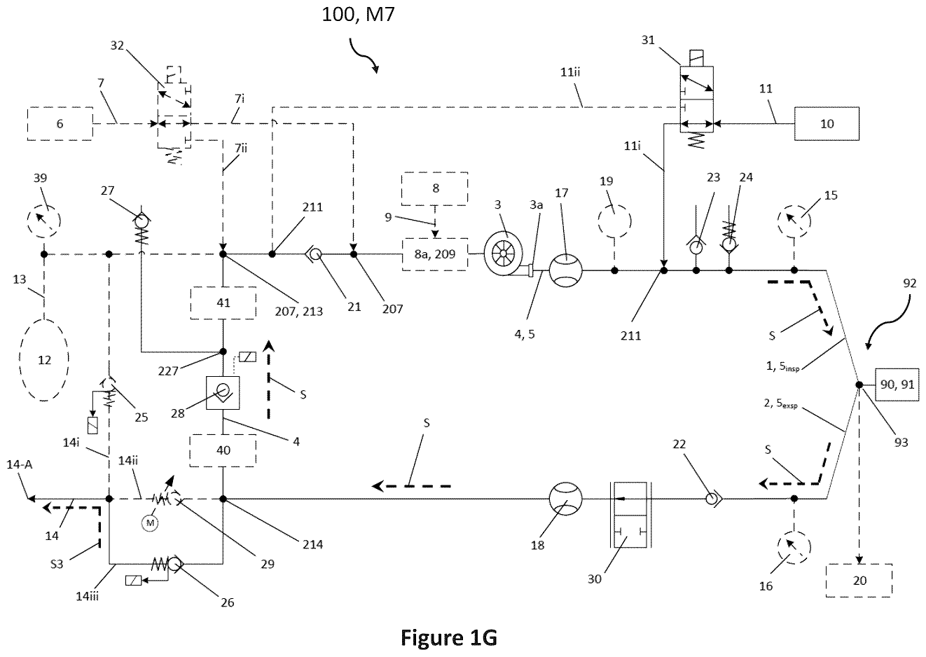

FIG. 1 shows a schematic structure of the device in a first exemplary embodiment FIGS. 1A to 1G show the mode of operation of the device in various operating modes based on the example of the first exemplary embodiment, wherein

FIG. 1A shows the device in a first operating mode for a manual ventilation with application of volatile anesthetics;

FIG. 1B shows the device in a second operating mode for an automatic ventilation with application of volatile anesthetics;

FIG. 1C shows the device in a third operating mode for a manual ventilation without application of volatile anesthetics;

FIG. 1D shows the device in a fourth operating mode for an automatic ventilation without application of volatile anesthetics;

FIG. 1E shows the device in a fifth operating mode for a manual ventilation in a battery and/or error mode;

FIG. 1F shows the device in a sixth operating mode for a constant flow (HFOT), wherein volatile anesthetics can optionally be applied;

FIG. 1G shows the device in a seventh operating mode, which represents a service mode;

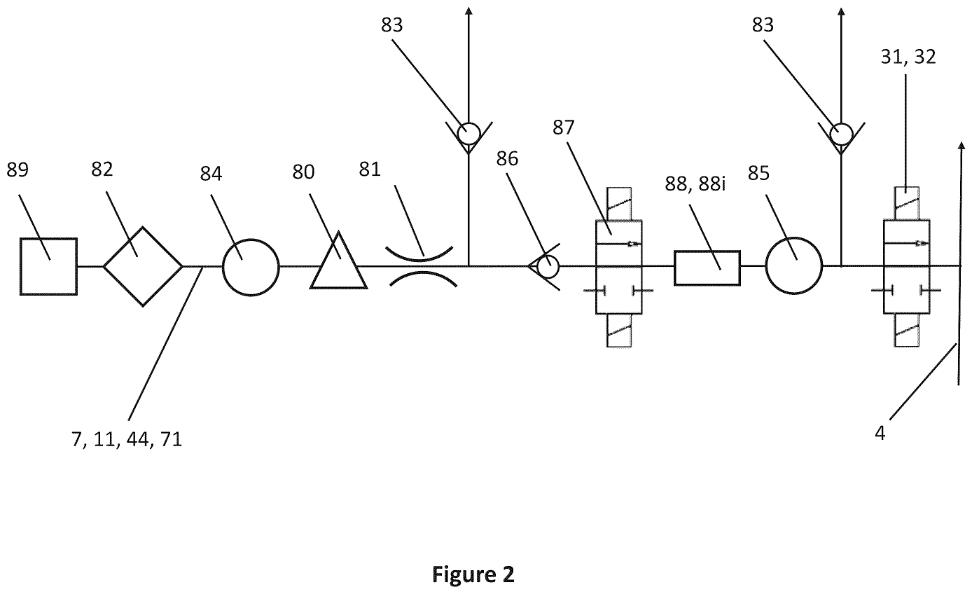

FIG. 2 schematically shows an exemplary structure of the supply lines

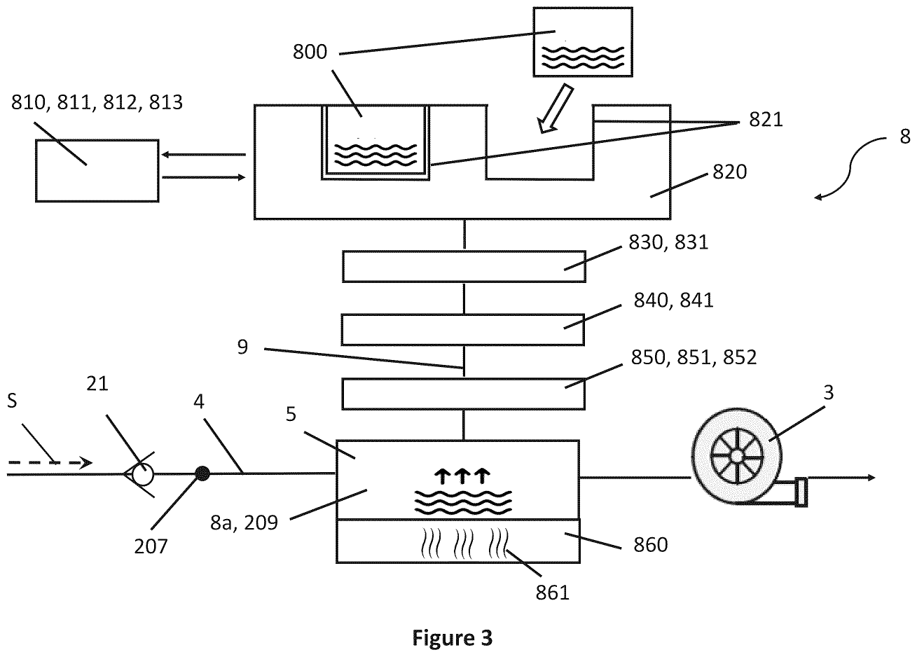

FIG. 3 schematically shows a portion of the breathing gas line, in which the vaporization element is arranged, which is connected via the anesthetic supply line to the anesthetic module.

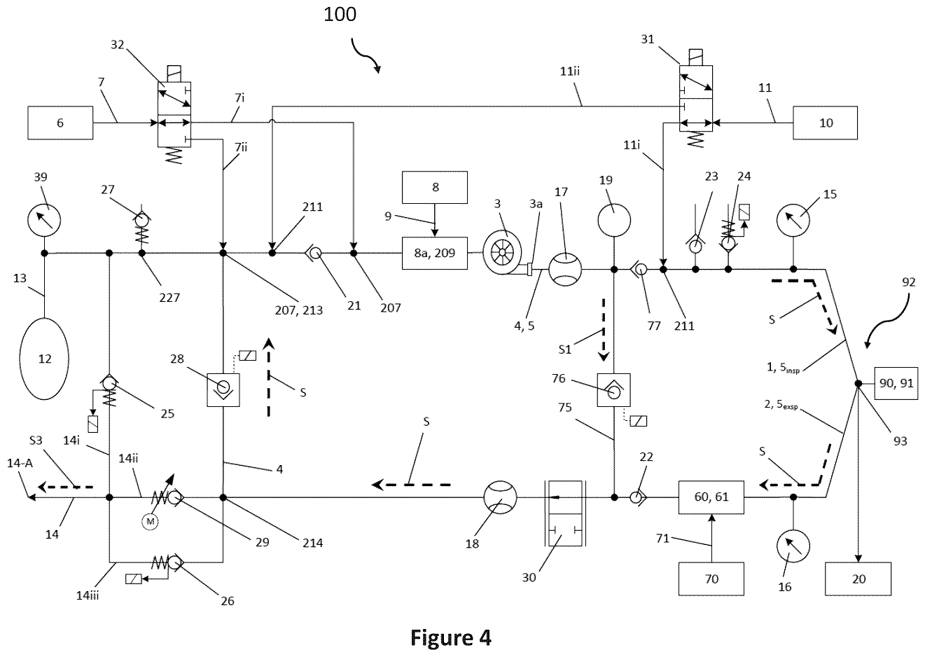

FIG. 4 shows a schematic structure of the device in a second exemplary embodiment

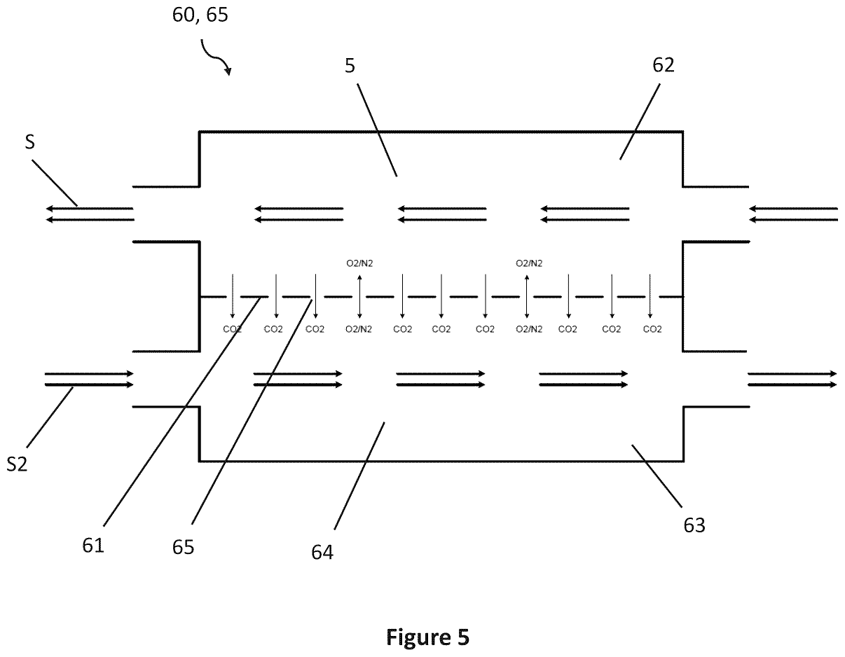

FIG. 5 shows a schematic structure of the mechanical separating means with diffusion filter.

FIG. 1 shows a schematic structure of the device 100 in a first exemplary embodiment. The device 100 according to the invention is configured for supplying breathing gas and comprises at least one breathing gas line 4 for conducting a breathing gas mixture 5, wherein the breathing gas line 4 comprises at least one outlet 14-A, via which the breathing gas mixture 5 can be at least partially, at least temporarily discharged, wherein the breathing gas line 4 comprises an inspiratory branch 1, which is designed to conduct breathing gas to a fitting 93 for a patient interface, wherein the breathing gas line 4 comprises an expiratory branch 2, which is designed to conduct breathing gas between the fitting 93 for a patient interface and the outlet 14-A. The device 100 is characterized in that at least one shutoff valve 28 is arranged in the breathing gas line 4, which is designed and configured to at least temporarily establish a connection conducting breathing gas from the expiratory branch 2 to the inspiratory branch 1.

The device 100 according to the invention can be an anesthesia workstation. The device 100 can be configured to ventilate and/or anesthetize a living being. For this purpose, the device 100 can be configured to provide and/or conduct and/or prepare and/or dispose of a breathing gas mixture or an anesthetic gas mixture. The device 100 is configured and designed to automatically and/or manually ventilate or anesthetize a living being. The device 100 comprises at least one breathing gas line 4 and at least one conduction system 14 for this purpose.

The device 100 has at least one fan unit 3. The fan unit 3 is configured and designed to generate a breathing gas flow for ventilating and/or anesthetizing a patient and to convey it if necessary in the direction of the patient. A breathing gas mixture 5 for ventilation and/or anesthesia can be conveyed using the fan unit 3. The fan unit 3 can therefore supply the conveying energy for the breathing gas mixture 5. The breathing gas mixture 5 can be normal breathing air from the surroundings or pure oxygen O2 or breathing air enriched with oxygen O2. In particular, the breathing gas mixture 5 can also contain at least one anesthetic A. The breathing gas mixture 5 can therefore also in particular be an anesthetic gas. The breathing gas flow can be conveyed from the ambient air and/or from pressurized gas cylinders and/or from a central gas system (CGS) of the hospital (not shown).

The fan unit 3 is preferably designed as at least one fan (blower) 3, which can comprise at least one fan wheel for generating a breathing gas flow. In some embodiments, multiple fans 3 can also be connected in succession or in parallel to one another. The device 100 can in alternative embodiments also have another technical unit instead of the fan, which can generate breathing work or conveying energy for the breathing gas mixture. In such a case, the fan unit 3 can comprise, for example, at least one driven folded bellows or at least one piston motor. In an alternative embodiment, the device 100 can therefore also be operated using a piston motor having, for example, multiple pistons and corresponding valves (not shown). In preferred embodiments, the fan unit 3 is designed as at least one fan 3. The fan unit 3 is therefore sometimes only referred to as a fan 3 hereinafter for the sake of simplicity—this does not exclude the above-mentioned exemplary embodiments having multiple fans, technical units, folded bellows, or piston motors. The device 100 can comprise at least one electrical drive (not shown) for operating the fan 3.

The device 100 can be supplied with power via at least one power source 103, 104 shown here. The device can preferably be supplied with energy via a primary power source 103 and alternatively via at least one secondary power source 104. For this purpose, the device 100 can have a grid plug and accumulators and/or capacitors arranged in the device interior.

The device 100 can be connected via a grid plug to the supply grid and therefore can be supplied with energy. Grid plug and supply grid can then be used as the primary power source 103. Alternatively or additionally, the device 100 can also be supplied with energy via the accumulators/capacitors. The accumulators/capacitors can then be used as a secondary power source 104. The device 100 can therefore be operated in grid operation and/or in battery operation.

The device 100 can comprise at least one display unit (not shown). The display unit can be configured as a monitor, for example as a touchscreen for the display and/or input of medical data. It is also possible to arrange multiple monitors.

The device 100 comprises at least one control unit 101 and at least one storage unit 102. The fan 3 is actuated via the control unit 101. In general, the control unit 101 controls the fan 3 as specified by the configurations stored in the storage unit 102.

For example, the control unit 101 sets a specific speed of the fan wheel or regulates the fan speed to a setpoint value. The fan 3 can specify a flow and/or a pressure and/or a volume of breathing gas in this way. In a preferred embodiment, the fan 3 can generate a defined flow. In a particularly preferred embodiment, the fan 3 can generate a pressure-independent flow. Flow and pressure can be specified decoupled from one another, so that the flow can be generated independently of the pressure. This offers the advantage that upon specification of the required flow, different pressure-controlled breathing patterns can be constructed. For example, the setpoint value of the flow can be set on the basis of treatment specifications and/or sensor signals.

The device 100 is in particular configured and designed to provide the breathing gas mixture 5 in the form of a patient flow. The patient flow is used to supply breathing gas to the patient. The patient flow can be dependent on the breathing phases of the patient.

Alternatively or additionally, the device 100 is configured and designed to provide a permanent byflow. The byflow can exist independently of the breathing phases of the patient. Patient flow and byflow can be formed parallel to one another in the breathing gas line 4.

The device 100 comprises at least one sensor 15, 16, 17, 18, 19, 20, 39. In a preferred embodiment, the device 100 comprises a plurality of sensors 15, 16, 17, 18, 19, 20, 39 (see below).

The fan 3 can be adaptively actuated. An adaptive actuation can take place, for example, based on ventilation parameters ascertained and analyzed during the ventilation. The storage unit 102 is configured to store ventilation-relevant parameters. These ventilation-relevant parameters can be ventilation-specific parameters 110 detected during the breathing gas supply and/or patient parameters 111 stored in the storage unit 102. The patient parameters 111 can be ascertained beforehand. The patient parameters 111 can also be ascertained during the ventilation. The stored patient parameters 111 can be selected from the following group: age, sex, weight, height, pre-existing conditions, body fat proportion, body mass index (BMI), ideal body weight (BWI), health status, nutrition status, metabolism of the patient, tidal volume, indirect calorimetry, and the like.

The fan 3 comprises at least one fan outlet 3a. The fan 3 discharges the breathing gas mixture 5 into the at least one breathing gas line 4 via the fan outlet 3a. The breathing gas mixture 5 is conveyed via the breathing gas line 4. The fan 3 specifies a main flow S of the breathing gas mixture 5 in this case, the direction of which is shown indicated by dashed lines in FIG. 1. The breathing gas line 4 comprises an inspiratory branch 1. Moreover, the breathing gas line 4 can comprise an expiratory branch 2. The breathing gas line 4 can moreover comprise a reservoir line 13.

The inspiratory branch 1 can be designed at least to conduct breathing gas to a fitting 93 for a patient interface. The expiratory branch 2 can be designed at least to conduct breathing gas between the fitting 93 for the patient interface and at least one outlet 14-A. A patient interface, via which a connection to a patient 90 can be established, can be connected to the fitting 93.

The reservoir line 13 can be viewed as part of the inspiratory branch 1. The reservoir 12 is pneumatically connected to the breathing gas line 4 via the reservoir line 13. Gases from the reservoir 12 can be introduced at at least one reservoir feed point 213. The breathing gas line 4 can be pneumatically connected to the conduction system 14 for conducting the breathing gas mixture 5.

The breathing gas line 4 having its inspiratory branch 1 and its expiratory branch 2 can be at least partially formed in the device. The breathing gas line 4 having its inspiratory branch 1 and its expiratory branch 2 can also be at least partially formed outside the device, for example in a tubing system.

In the specific exemplary embodiment according to the figures, the breathing gas line 4 comprises the reservoir line 13, the inspiratory branch 1, and the expiratory branch 2, which are pneumatically connected to one another. The breathing gas line 4 is moreover pneumatically connected to the conduction system 14.

To introduce the breathing gases and/or anesthetics to provide the breathing gas mixture 5, the breathing gas line 4 is pneumatically connected to at least one fresh gas supply line 7 and/or to at least one O2 flush supply line 11 and/or to at least one anesthetic supply line 9 and/or a nitrogen oxide supply line 44. The supply lines 7, 9, 11, 44 generally open in this case into the inspiratory branch 1 of the breathing gas line 4. Breathing gases and/or anesthetics are fed into the breathing gas line 4 via the supply lines 7, 9, 11, 44. It is ensured via check valves in the supply lines 7, 9, 11, 44 that the breathing gas mixture 5 cannot be discharged via the supply lines 7, 9, 11, 44.

The fan 3 is preferably arranged in the inspiratory branch 1. The fan 3 is preferably arranged downstream after the expiratory branch 2. In general, inspiratory breathing gas 5ᵢₙₛₚ is conveyed in the inspiratory branch 1. Fresh gas and/or oxygen and/or anesthetic can be introduced into the inspiratory branch. At least the fan 3 for specifying the breathing gas flow S can be arranged in the inspiratory branch. The inspiratory breathing gas 5ᵢₙₛₚ. can be conveyed via the patient interface to the patient 90 via the inspiratory branch 1.

In general, expiratory breathing gas 5ₑₓₛₚ is conveyed in the expiratory branch 2. The patient 90 can emit expiratory breathing gas 5ₑₓₛₚ. via the patient interface into the expiratory branch 2. The expiratory breathing gas 5ₑₓₛₚ. can be conducted away from the patient via the expiratory branch 2. The expiratory breathing gas 5ₑₓₛₚ. can be conveyed to a device 40 for separating at least CO2 via the expiratory branch 2. The expiratory branch 2 can extend at least from the patient interface to the separating means 40, 60.

The main flow S of the breathing gas mixture 5 can run from the fan outlet 3a along the inspiratory branch 1 to a fitting 93 for a patient interface. The breathing gas mixture 5 can run along the expiratory branch 2 back to the inspiratory branch 1 from the fitting 93. For example, the breathing gas mixture 5 can run along the expiratory branch 2 to the fan inlet (not shown in more detail here) of the fan 3. The breathing gas can thus remain in at least one—substantially—closed first circuit K1.

Inspiratory branch 1 and expiratory branch 2 can therefore form at least the first circuit K1, in which the breathing gas mixture 5 can be conducted. In this case, the breathing gas mixture 5 can be able to be at least partially discharged via the outlet 14-A. The breathing gas mixture 5 can be able to be conducted in the first circuit K1 with the main flow S. In addition to a patient flow, a permanent byflow can advantageously also flow in the circuit K1.

The byflow offers the advantage that the fan 3 does not have to be stopped in operation. The fan 3 always rotates at a minimum speed. A permanent flow prevails in the breathing gas line 4 due to the byflow.

A patient interface 91, via which the patient is supplied with the breathing gas mixture 5, can be connected at the fitting 93. The breathing gas mixture 5 can be supplied to the patient 90 via the inspiratory branch 1. For this purpose, the device 100 has at least one interface for coupling a tubing system 92. The breathing gas mixture 5 can be supplied to the patient 90 via the tubing system 92. For this purpose, a patient interface 91 can be connected to the tubing system 92.

The tubing system 92 can be a two-tube system and can have at least one inspiration tube and at least one expiration tube. In this case, the patient 90 can be supplied with the breathing gas mixture 5 via the inspiration tube of the tubing system 92 and the patient interface 91 and can exhale via the expiration tube of the tubing system 92. The exhalation gas can therefore be returned to the device 100 via the expiration tube and at least one closed circuit can exist. A closed circuit exists if the exhalation gas—after preparation—is used for the inspiration again. If a two-tube system is used, the fitting 93 can be designed as a Y-piece, via which the inspiration tube and the expiration tube are connectable.

In some embodiments, the tubing system 92 can also be a one-tube system and can only have an inspiration tube. In this case, the patient 90 can be supplied with the breathing gas mixture 5 via the tubing system 92 and the patient interface 91 and can exhale into the surroundings. An open circuit without an expiratory branch can thus exist.

In some embodiments, the tubing system 92 can also be a two-tube system and can have an inspiration tube and an expiration tube without the breathing gas running in a closed circuit. This system is referred to herein as a semi-open circuit. A semi-open circuit in the sense of the invention means that the patient 90 can be supplied with the breathing gas mixture 5 via the inspiration tube of the tubing system 92 and the patient interface 91 and can exhale via the expiration tube of the tubing system 92, wherein the exhalation gas is emitted to the surroundings via the expiration tube.

The device 100 is configured and designed to form a closed circuit and/or a semi-open circuit and/or an open circuit depending on the use of the tubing system and depending on the settings in the device 100. The device 100 is therefore to be used flexibly, since the breathing gas line 4 can form a closed circuit and/or a semi-open circuit and/or an open circuit. Depending on the application, it is possible to change between a closed and a semi-open and an open circuit.

A closed circuit in the sense of the invention means that the breathing gas mixture 5 is guided in a circuit in the main flow S, wherein oxygen O2 and/or anesthetics and/or other gases or gas components or substances can be supplied or withdrawn.

A gas exchange takes place in the lungs of the patient. Oxygen (O2) is absorbed in the blood and carbon dioxide (CO2) is eliminated. The patient therefore withdraws gas or gas components from the circuit and adds new and/or modified gases or gas components to the circuit. Therefore, gases or gas components permanently have to be discharged and/or supplied from or to the breathing gas mixture guided in the circuit. For example, oxygen O2 and/or anesthetics and/or other gases can be readjusted depending on use. Carbon dioxide (CO2) and/or other undesired gas components can be withdrawn from the circuit. The gas components can therefore be used and/or withdrawn and disposed of in a defined manner.

The control unit 101 and/or a user can regulate/control the proportions of the components of the breathing gas mixture 5 in this case. For this purpose, fresh gas and/or oxygen O2 and/or anesthetics and/or CO2 are introduced or discharged or separated.

The device 100 can comprise at least one fitting PAUX for pneumatic accessories (not shown). The device 100 preferably comprises more than one fitting, for example two or three or 4 or more. The fittings PAUX can be configured and designed to connect accessories relevant to ventilation and/or anesthesia to the device 100. These accessories can be selected from the following group: larynx mask, tracheal tube, cuff, esophageal catheter, bladder catheter. These accessories can execute their function with the aid of a balloon, which can be supplied with flow and/or pressure and/or volume via the PAUX fittings. The PAUX fittings can be configured and designed such that the functions of the connected accessories can be checked automatically. For this purpose, the device 100 can be designed to supply or discharge a flow and/or pressure to or from the PAUX fittings. Furthermore, sensors can be connected to the PAUX fittings in order to check the functionality of the accessories.

In order to be able to monitor the gas composition of the breathing gas mixture 5, the device 100 can comprise at least one sensor 19. The at least one sensor 19 is arranged in and/or on the breathing gas line 4. The sensor 19 can detect, for example, the oxygen concentration and/or the CO2 concentration (capnometry) and/or the concentration of one or more anesthetics and/or the moisture. The sensor 19 can accordingly be designed as an oxygen sensor and/or CO2 sensor and/or anesthetic gas sensor and/or as a moisture sensor.

The device 100 can alternatively or additionally also comprise a multigas sensor 20. The multigas sensor 20 is configured and designed to detect different gas components in parallel. In one preferred embodiment, the device 100 comprises at least one multigas sensor 20.

The sensors 19 and/or the multigas sensor 20 are arranged on at least one point of the breathing gas line 4. The sensors 19 and/or the multigas sensor 20 detect the gas composition of the breathing gas mixture 5. The sensors 19 and/or the multigas sensor 20 can be arranged in the inspiratory branch 1 and/or in the expiratory branch 2 of the breathing gas line 4.

In one exemplary embodiment according to the figures, at least one multigas sensor 20 can be arranged directly before the patient at or close to the patient interface. For example, the multigas sensor 20 can be connected to the Y piece 93 of the tubing system 92. The multigas sensor 20 can take a sample gas at the Y piece 93 in order to be able to measure inspiratory and expiratory values.

In some embodiments, a switching valve can be provided at the multigas sensor 20 in order to be able to measure the concentration of the anesthetics directly at the vaporization point of the anesthetics. This has a safety aspect if the anesthetic has to be changed. However, it can also be used to accelerate the regulation of the concentration of the anesthetic. This is advantageous since the total gas volume of device and lungs is very large (8-9 L) and the anesthetic has to be mixed in.

In some embodiments, the sensors 19 can also be arranged at multiple points of the breathing gas line 4 in order to monitor the gas composition of the breathing gas mixture 5 in detail. For example, CO2 sensors and/or O2 sensors can be arranged before and after the patient and before and after a device for CO2 separation, namely a chemical separating means 40 and/or a mechanical separating means 60 (see below). The functionality of the separating means 40, 60 can thus be monitored.

In one exemplary embodiment according to the figures, at least one oxygen sensor 19 can be arranged directly after the fan 3 and can detect the oxygen concentration.

The components of the breathing gas mixture 5 are controlled or regulated by the control unit 101. Saved instructions and/or preset treatment conditions and/or user specifications and/or the detected sensor signals can be taken into consideration for this purpose. The user specifications can be carried out manually by the user, for example by trained medical personnel. The user specifications can take place beforehand and/or under the ventilation/anesthesia.

The control unit 101 takes into consideration in particular the detected sensor signals. In particular, the sensor signals of the at least one CO2 sensor 19 and/or O2 sensor 19 and/or anesthetic gas sensor 19 and/or multigas sensor 20 are taken into consideration. The control unit 101 can thus be capable of adaptively adjusting the components of the breathing gas mixture 5 to the respective situation under ventilation or under anesthesia.

In general, the breathing gas mixture 5, after it has flowed through the sensors 19 or the multigas sensor 20 and has been measured, returns to the circuit of the breathing gas line 4. However, it is also possible that breathing gas mixture 5 is withdrawn from the circuit in order to examine it for its gas components without the gas subsequently being returned to the circuit. This can be advantageous to make the evaluation of the measurement signals more sensitive.

The device can preferably comprise at least one flow sensor 17, 18 and/or at least one pressure sensor 15, 16, 39. The device 100 can comprise at least one inspiratory flow sensor 17. The at least one inspiratory flow sensor 17 can be arranged, for example, downstream from the fan 3 in the inspiratory branch 1 of the breathing gas line 4. In the exemplary embodiment according to FIG. 1, the inspiratory flow sensor 17 is arranged between the fan 3 and the O2 flush supply line 11. The inspiratory flow sensor 17 is configured and designed to measure at least one inspiratory flow.

Alternatively or additionally, the device 100 can comprise at least one inspiratory pressure sensor 15. The at least one inspiratory pressure sensor 15 can be arranged, for example, downstream of the fan 3 in the inspiratory branch 1 of the breathing gas line 4. In the exemplary embodiment according to FIG. 1, the inspiratory pressure sensor 15 is arranged directly before the patient 90 or the patient interface. The inspiratory pressure sensor 15 is configured and designed to measure at least one inspiratory pressure.

Alternatively or additionally, the device 100 can comprise at least one pressure sensor 39. The pressure sensor 39 can be arranged, for example, in or on the reservoir line 13. In the exemplary embodiment according to FIG. 1, the pressure sensor 39 is arranged adjacent to the reservoir 12 in the reservoir line 13. The pressure sensor 39 is configured and designed to measure at least one pressure of the reservoir 12. The bag pressure of the reservoir 12 can be detected via the pressure sensor 39. The pressure sensor 39 can transmit the detected pressure values to the control unit 101 in order to monitor the pressure of the reservoir 12.

Furthermore, the device 100 can comprise at least one expiratory flow sensor 18. The at least one expiratory flow sensor 18 can be arranged, for example, in the expiratory branch 2 of the breathing gas line 4. The expiratory flow sensor 18 is configured and designed to measure at least one expiratory flow.

Alternatively or additionally, the device 100 can comprise at least one expiratory pressure sensor 16. The at least one expiratory pressure sensor 16 can be arranged, for example, in the expiratory branch 2 of the breathing gas line 4. In the exemplary embodiment according to FIG. 1, the expiratory pressure sensor 16 is arranged directly after the patient 90 or the patient interface. The expiratory pressure sensor 16 is configured and designed to measure at least one expiratory pressure.

The flow sensors 17, 18 and/or pressure sensors 15, 16, 39 and/or sensors 19, 20 are designed as measuring units and detect at least one ventilation-specific parameter 110 which are selected from the following group: inspiratory patient pressure, inspiratory patient flow, inspiratory tidal volume, inspiratory minute volume, inspiratory breathing rate, inspiratory O2 concentration, inspiratory CO2 concentration, inspiratory N2O concentration, inspiratory anesthetic gas concentration; expiratory patient pressure, expiratory patient flow, expiratory tidal volume, expiratory minute volume, expiratory breathing rate, expiratory O2 concentration, expiratory CO2 concentration, expiratory N2O concentration, expiratory anesthetic gas concentration; gas temperature, gas moisture, leakage, or the like. The ventilation-specific parameters 110 to be detected are not restricted to these examples.

The control unit 101 is configured and designed to adaptively regulate the mode of operation of the device 100 depending on ascertained and analyzed ventilation parameters.

Measured values and parameters of accessories such as cuff pressure (sealing collar tube), pressure of a stomach or bladder catheter, pressure of the seal of the larynx mask, leakage, resistance, bag pressure, or the like can also be detected and incorporated here.

The control unit 101, which is in communication with the measuring units, can define the technical ventilation parameters and in particular also adaptively adjust them on the basis of these ventilation-specific parameters 110. The technical ventilation parameters also comprise, in addition to the pressure or flow or volume, the supply of fresh gas and/or oxygen O2 and/or anesthetics.

A possible leakage can be detected and transmitted to the control unit 101 on the basis of the measuring units. The device 101 can comprise an alarm generator which can trigger an alarm signal upon the detection of a leakage.

The device 100 can have at least one reservoir 12 and at least one reservoir line 13. The reservoir 12 can be designed, for example, as a manual bag. The reservoir 12 can alternatively or additionally also be designed as a breathing bellows. The reservoir 12 can supply the conveying energy for the breathing gas mixture 5. The reservoir 12 can in particular be operated manually by a user. For example, a user can operate the reservoir 12 by compression so that conveying energy is provided for the breathing gas mixture 5.

The reservoir 12 is configured and designed, among other things, to provide a volume for the breathing gas mixture 5 for the purpose of pressure monitoring or pressure compensation. For this purpose, the reservoir 12 is pneumatically connected via the reservoir line 13 to the breathing gas line 4. The reservoir 12 can be used both as a pressure source and as a pressure sink. The reservoir 12 can also be used both as a volume source and as a volume sink.

The reservoir line 13 is configured such that a flow of the breathing gas mixture 5 in the direction toward the reservoir 12 is possible. The reservoir line 13 is moreover also configured such that a flow of the breathing gas mixture 5 in the direction away from the reservoir 12 is possible. During an inspiration, the reservoir 12 can be used as a pressure and/or volume source. During an expiration, the reservoir 12 can be used as a pressure and/or volume sink.

The reservoir 12 comprises, for example, at least the volume of one breath. For example, the reservoir 12 comprises at least 250 mL, preferably at least 500 mL, particularly preferably at least 1 L. In a specific exemplary embodiment, the reservoir comprises between 1 L and 5 L, for example between 2 L and 2.5 L.

The reservoir 12 is configured and designed, among other things, to provide a volume for conveyance to the fan 3. A large part of the breath volume conveyed to the patient 90 can be conveyed from the reservoir 12 via the fan 3 to the patient 90. A gas exchange takes place in the lung during the respiration of the patient 90, due to which the volume per breath decreases. Therefore, volume always has to be added to the closed circuit in order to at least replace the oxygen used.

In some embodiments, at least one of the O2 flush supply lines 11 of the device 100 can be arranged such that the reservoir 12 can be filled directly with oxygen. This can form an additional safety level, since in this way the reservoir 12 can be filled rapidly and directly with a large amount of oxygen and/or an oxygen mixture. This can be advantageous in particular if the device 100 is operated in a TIVA mode and/or a HFOT mode.

In some embodiments, the device 100 can comprise at least one pressure sensor 39. The pressure sensor 39 can be configured and designed to detect the pressure of the reservoir 12. The pressure sensor 39 is preferably arranged in or on the reservoir line 13. The pressure sensor 39 can be configured and designed to detect the pressure of the reservoir 12 and transmit it to the control unit 101. The fill level of the reservoir 12 can be detected and monitored via the pressure sensor 39.

A maximum and/or a minimum pressure of the reservoir 12 can be stored in the control unit 101. If the pressure in the reservoir 12 falls below a critical level and/or the pressure in the reservoir 12 rises above a critical level, an action can be triggered. For example, an alarm can be triggered. For this purpose, the device 100 can comprise an alarm generator, which is controlled by the control unit 101 (not shown). The alarm can signal to the control unit 101 and/or the user, thus the trained medical personnel, that the pressure in the reservoir 12 has to be increased or reduced.

If the pressure falls below the minimal pressure of the reservoir 12, pressure can be built up in the reservoir 12. For example, the fresh gas module 6 and/or the O2 flush 10 can be actuated or operated such that fresh gas and/or oxygen is introduced into the reservoir 12 to increase the pressure in the reservoir 12.

If the pressure exceeds the maximum pressure of the reservoir 12, pressure can be discharged from the reservoir 12. For this purpose, the reservoir line 13 is pneumatically connected to the conduction system 14. The conduction system can comprise at least one overflow valve 25, via which pressure can be emitted from the reservoir 12. Overfilling of the reservoir 12 can thus be prevented.

The device 100 can comprise the at least one conduction system 14, via which excess gases, for example excess breathing gas mixture 5, can be discharged. The conduction system 14 can be configured and designed for discharging at least a part of the breathing gas mixture 5 for the purpose of pressure monitoring or pressure compensation. The conduction system 14 can be configured and designed to regulate the oxygen and/or anesthetic concentration of the breathing gas mixture 5.

The conduction system 14 is designed as a gas-carrying line and is pneumatically connected to the breathing gas line 4. The conduction system 14 comprises at least one outlet 14-A. The conduction system 14 can comprise one or more lines, which all open into the same outlet 14-A (see FIG. 1). Multiple lines having multiple outlets 14-A are also possible (not shown). Breathing gas mixture 5 can be conducted with a flow S3 via the conduction system 14. The direction of the flow S3 always runs from the breathing gas line 4 in the direction of the outlet 14-A.

In the specific exemplary embodiment according to FIG. 1, the conduction system 14 comprises, for example, three lines, namely the first line 14i, the second line 14ii, and the third line 14iii. The lines 14i, 14ii, 14iii are each pneumatically connected at least to the breathing gas line 4 and each open into the outlet 14-A. The breathing gas mixture 5 can be discharged on at least three different paths from the breathing gas line 4 in this way. Multiple lines and multiple outlets 14-A are conceivable. The device 100 is not restricted to the exemplary embodiments shown.

Via the outlet 14-A, the breathing gas mixture 5 can be discharged into the surroundings or remain in the circuit. The conduction system 14 can optionally have a pump (not shown) for this purpose, which can conduct discharged breathing gas mixture 5 from the outlet 14-A via a line (not shown) back to the fresh gas module 6.

The outlet 14-A can comprise at least one filter 94 (not shown). In preferred embodiments, the outlet 14-A can comprise a double filter system. Multiple filters 94 are also conceivable. The filters 94 are preferably arranged replaceably at the outlet 14-A. The discharged breathing gases can be filtered via the filter 94 at the outlet 14-A, so that substances which are potentially harmful to the environment or to health can be prevented from reaching the surroundings in unfiltered form.

The filter 94 can comprise activated carbon, for example. Activated carbon can function as an absorbent for volatile anesthetics and/or the metabolites thereof and/or other substances. The filters 94 can therefore be designed as activated carbon filters, using which volatile anesthetics VA and/or the metabolites thereof and/or other substances potentially harmful to the environment or health can be filtered out of the discharged breathing gas mixture 5. The absorbed substances are enriched due to absorption in the activated carbon until they have to be replaced or cleaned in order to produce the filter property again.

At least one sensor 95 (not shown) can be arranged in or at the outlet 14-A in preferred embodiments. The sensor 95 is preferably arranged after the filter 94 in the flow direction. The sensor 94 can be designed, for example, as an optical sensor and can be configured to detect the anesthetic concentration of the discharged breathing gas mixture 5 and/or to transmit it to the control unit 101. The filter properties of the activated carbon filters can therefore be determined in this way.

In an exemplary embodiment, a first filter 94i and a second filter 94ii can be arranged in or at the outlet 14-A. The first filter 94i and the second filter 94ii can form a double filter system having magazine function, which can effectively prevent an escape of anesthetics. In a double filter system, for example, two activated carbon filters can be arranged in succession. Sensors 95 can be arranged before and/or after a first filter 94i. Alternatively or additionally, sensors can also be arranged before and/or after the second filter 94ii. The sensors can then detect the respective anesthetic concentrations before and after the filters. If the anesthetic gas concentration after the first filter increases above a predefined limiting value, the second filter can continue to filter the breathing gas mixture.

Such a double filter system having sensors offers the advantage that the first filter only has to be replaced when it can detectably no longer filter anesthetics from the breathing gas. The second filter still ensures the effective filtering of the breathing gas mixture. Costs for disposal and filter material can be saved.

The conduction system 14 can therefore be configured and designed to dispose of the anesthetic gas in a controlled manner and/or prepare it again for renewed use.

The device 100 is configured and designed to be able to guide the breathing gas mixture 5 in a closed circuit. This offers the advantage that the volatile anesthetics VA, which are usually harmful to the environment and climate, remain in the system and are not discharged in an uncontrolled manner to the surroundings. Furthermore, the device 100 according to the invention enables a frugal and therefore cost-effective use of anesthetics, since unconsumed anesthetics are not discharged into the surroundings, but rather can be reused.

Since the expiration air of the patient 90 remains in the breathing gas line 4 of the closed circuit, there is the necessity of withdrawing carbon dioxide (CO2) from the breathing gas mixture 5. The device 100 therefore has at least one separating means 40, 60 for separating CO2 from the breathing gas mixture 5.

In a first exemplary embodiment according to FIG. 1, the device comprises the at least one separating means 40. The separating means 40 can be designed as a chemical CO2 absorber. The separating means 40 can therefore contain one or more chemically active absorbents, which bind CO2. The separating means 40 is also referred to herein as a chemical separating means 40. The separating means 60 is also referred to herein as a mechanical separating means 60 (see below).

Chemical absorbents can be selected from the following group: calcium hydroxide, sodium hydroxide, potassium hydroxide, barium hydroxide. For example, a mixture made up of one or more of these components in the form of soda lime is used, through which the breathing gas mixture 5 is conducted after the expiration. In the chemical reaction between the chemical absorbents and CO2, the CO2 is bound and water (H2O) results. The reaction is exothermic, so that temperatures between 6° and 70° C. can develop. The chemical absorbents partially also bind anesthetics, so that the anesthetic concentration is to be checked after the separating means 40, so that the anesthetic can be additionally dosed depending on use. The CO2 binding of the chemical separating means 40 is very sensitive and is active even at very low CO2 concentrations of the breathing gas mixture 5.

Since water precipitates in the chemical absorption of CO2 in the chemical absorber, the breathing gas mixture 5 is enriched with H2O after it has passed through the chemical separating means 40. For this reason, the device 100 can comprise a moisture control module 41. The moisture control module 41 is configured and designed to control the moisture of the breathing gas mixture 5. The moisture control module 41 can be configured, for example, as a cold trap and can contain a water reservoir in which excess precipitated water can be collected and disposed of.

The device 100 can optionally comprise one or more moisture sensors (not shown). Moisture sensors are advantageous in particular if the device 100 is operated using a chemical separating agent 40. The moisture sensors can be configured and designed to determine the moisture of the breathing gas mixture 5 and to give feedback to the control unit 101 and/or to the moisture control module 41. The moisture sensors can preferably be arranged in the inspiratory branch 1 and can determine the moisture in the inspiratory breathing gas mixture 5.

The device 100 can have at least one module 6, 8, 10 for introducing fresh gas and/or oxygen O2 and/or anesthetics. The modules 6, 8, 10 are each connected via at least one supply line 7, 9, 11 to the breathing gas line 4. The supply lines can be pneumatically connected to the breathing gas line 4, wherein the supply lines can be permanently open or can be opened and/or closed in a manner controlled via valves. The modules 6, 8, 10 can be used as a pressure and/or volume source and the breathing gas line 4 can be used as a pressure and/or volume sink.

The device 100 can comprise at least one fresh gas module 6 and at least one fresh gas supply line 7. Fresh gas can be added to the breathing gas mixture 5 via the fresh gas supply line 7. The fresh gas supply line 7 is pneumatically connected to the breathing gas line 4 for this purpose. The fresh gas can be introduced at at least one fresh gas feed point 207.

Fresh gas in the meaning of the invention comprises any fluid, breathing gas, and/or gas mixture which is suitable for and can be used for breathing, ventilation, and/or respiratory therapy. Fresh gas can be, for example, conveyed ambient air or a gas mixture made up of the ambient air and an auxiliary gas or an auxiliary gas alone. The fresh gas can be conveyed directly from the ambient air and/or can be acquired from pressurized gas cylinders and/or from a central gas system (CGS) of the hospital. Fresh gas can also be oxygen or air enriched with oxygen. In some embodiments, the fresh gas can also contain at least one anesthetic gas.

The fresh gas supply line 7 generally opens into the inspiratory branch 1 of the breathing gas line 4. The fresh gas can be introduced into the breathing gas line 4 upstream before the fan 3 or downstream after the fan 3.

An oxygen-containing gas mixture and/or oxygen can be introduced into the breathing gas line 4 via the fresh gas module 6 in order to maintain or keep controlled the oxygen concentration of the breathing gas mixture 5. The breathing gas mixture 5 is conveyed in the breathing gas line 4 so that it can be supplied with fresh gas via the fresh gas module 6 and thus can be enriched with oxygen.

The oxygen content of the breathing gas mixture 5 is controlled or regulated by the control unit 101. The oxygen content of the breathing gas mixture 5 can be controlled or regulated automatically on the basis of the detected sensor signals. The oxygen content of the breathing gas mixture 5 can in particular also be controlled or regulated manually by a user, for example by trained medical personnel. The trained medical personnel can thus supply fresh gas or oxygen as needed to the patient at any time under the ventilation and/or anesthesia.

The oxygen introduction can be regulated such that the oxygen concentration of the breathing gas mixture 5 is generally in a range between 10 and 60%, preferably between 20 and 50%. An oxygen saturation of 40 to 50% can generally be used for continuous ventilation and/or anesthesia.

In preferred embodiments according to the figures, the fresh gas is introduced upstream before the fan 3 into the breathing gas mixture 5. The at least one fresh gas supply line 7 then opens upstream before the fan 3 into the breathing gas line 4.

In some embodiments, the fresh gas can be introduced at more than one point into the breathing gas line 4, for example, at at least two points. For example, the device 100 can be configured such that the fresh gas module 6 can be connected via at least two supply lines 7i, 7ii to the breathing gas line 4.

In the specific embodiment according to FIG. 1, the fresh gas supply line 7 can divide into at least two fresh gas supply lines 7i, 7ii, namely, for example, a first fresh gas supply line 7i and a second fresh gas supply line 7ii.

The first fresh gas supply line 7i and the second fresh gas supply line 7ii can be pneumatically connected to the breathing gas line 4. The first fresh gas supply line 7i and the second fresh gas supply line 7ii can preferably open at different points into the breathing gas line 4.

In some embodiments, the fresh gas supply lines 7i, 7ii can each simultaneously introduce fresh gas into the breathing gas line (not shown).

In preferred embodiments, the fresh gas supply lines 7, 7i, 7ii can be regulated via at least one fresh gas switching valve 32 such that in each case only one of the fresh gas supply lines 7i or 7ii can introduce fresh gas into the breathing gas line.

It can be regulated via the fresh gas switching valves 32 via which of the fresh gas supply lines 7, 7i, 7ii fresh gas is introduced into the breathing gas line 4. The location of the feed of fresh gas into the breathing gas line 4 can therefore be regulated via the fresh gas switching valves 32.

The fresh gas switching valves 32 can be electronically and/or mechanically switched. The fresh gas switching valves 32 can be switched automatically by the control unit 101 and/or manually by the user.

The fresh gas switching valve 32 can be designed as a monostable or as a bistable way valve. In preferred embodiments, the fresh gas switching valve 32 is designed as a monostable way valve. For example, the fresh gas switching valve 32 comprises a solenoid and a spring, using the interaction of which the valve can be switched. Energizing the solenoid actively guides the valve 32 into a first switching position. The solenoid is inactive when deenergized, so that the spring relaxes and brings the valve into the second switching position. The valve 32 is in its base position when deenergized. It is apparent from FIG. 1 that fresh gas is introduced via the first fresh gas supply line 7i when the valve 32 is in its base position, thus is deenergized. The second fresh gas supply line 7ii is operated when the valve 32 is energized.

In the exemplary embodiment according to FIG. 1, the fresh gas module 6 is connected to the fresh gas supply line 7. The fresh gas supply line 7 is connected to the fresh gas switching valve 32. The fresh gas switching valve 32 can be designed, for example, as a 2/3-way valve and can therefore have three fittings and two switching positions. The fresh gas switching valve 32 can then moreover be connected to the first fresh gas supply line 7i and to the second fresh gas supply line 7ii and depending on the switching position can supply either the first fresh gas supply line 7i or the second fresh gas supply line 7ii with fresh gas from the fresh gas module 6.

In the exemplary embodiment according to FIG. 1, the first fresh gas supply line 7i and the second fresh gas supply line 7ii open at different points into the breathing gas line 4. The device 100 can therefore be configured to regulate the location of the fresh gas introduction into the breathing gas line 4.

For example, the first fresh gas supply line 7i can open into the breathing gas line 4 in the flow direction before the first check valve 21. For example, the second fresh gas supply line 7ii can open into the breathing gas line 4 in the flow direction after the first check valve 21. For example, the second fresh gas supply line 7ii can in particular open into the breathing gas line 4 between the first check valve 21 and the vaporization element 8a (FIG. 1).

In alternative exemplary embodiments, it is also conceivable that the device 100 comprises at least two fresh gas 2/2-way valves instead of the one fresh gas 2/3-way valve 32. For example, the first fresh gas supply line 7i can comprise a first fresh gas way valve, which can be designed as a 2/2-way valve having two fittings and two switching positions. For example, the second fresh gas supply line 7ii can comprise a second fresh gas way valve, which likewise can be designed as a 2/2-way valve having two fittings and two switching positions (not shown).

The 2/2-way valves can thereafter be switched in parallel by the control unit 101 so that either the first fresh gas supply line 7i or the second fresh gas supply line 7ii can be opened to introduce fresh gas into the breathing gas mixture 5.

Alternatively or additionally, the device 100 can comprise at least one oxygen module 10. The oxygen module 10 can be designed as an O2 flush 10 and can comprise at least one O2 flush supply line 11. Oxygen O2 or an oxygen-containing gas mixture can be introduced into the breathing gas line 4 via the O2 flush supply line 11. The O2 flush supply line 11 is pneumatically connected for this purpose to the breathing gas line 4. The oxygen can be introduced at at least one O2 flush feed point 211.

The oxygen can be acquired from pressurized gas cylinders and/or from a central gas system (CGS) of the hospital. The device 100 is preferably connected to the central gas system (CGS) and additionally has fittings on at least one pressurized gas cylinder. The oxygen can therefore be acquired either from the central gas system (CGS) or from pressurized gas cylinders. The oxygen supply can thus be ensured at least over a certain period of time via the pressurized gas cylinders in the event of a failure of the central gas supply.

The O2 flush 10 can be designed and configured so that a user, for example trained medical personnel, can fill the breathing gas line 4 (the circuit part) and/or the reservoir 12 rapidly with oxygen.

The O2 flush 10 can be used:
to refill the breathing gas line 4 which has run empty after a leak and/or the reservoir 12
to wash anesthetics out of the lungs of the patient
to ventilate the patient for a certain time using pure oxygen

In particular, the O2 flush of the oxygen module 10 is designed and configured to flood the breathing gas line 4 and/or the reservoir 12 and/or the lungs of the patient with oxygen rapidly in emergency situations. The flow rate of the oxygen or the oxygen-containing gas mixture can in this case be, for example, 60 L/min. The flow rate is advantageously particularly high so that the breathing gas line 4 and/or the reservoir 12 can be refilled again quickly after a leak and/or the anesthetic can be flushed out quickly.

The breathing gas line 4 and/or the reservoir 12 can run empty intentionally or unintentionally due to a leak, for example after a hose change, a filter change, a sensor exchange, or an improper use of the tubing system 92 or the patient interface 91. In such cases, the loss of breathing gas mixture 5 in the breathing gas line 4 and/or the reservoir 12 cannot be refilled rapidly enough using the normal fresh gas flow. The O2 flush then offers a rapid option for refilling the breathing gas line 4 and/or the reservoir 12 with a breathable gas.

Furthermore, patients can be ventilated using undiluted oxygen via the O2 flush as needed at least for a short time, for example for several breaths. The patient can be supplied with at least 90% oxygen via the O2 flush, preferably with at least 93% oxygen. Via the O2 flush of the oxygen module 10, the oxygen can be fed undiluted into the breathing gas line 4; as it comes from the supply (namely the central gas system or the pressurized gas cylinder).

The oxygen introduction via the O2 flush 10 can be regulated such that the oxygen concentration of the breathing gas mixture 5 is up to 100% at least for a defined short time span. Ventilation using up to 100% oxygen over a short time span can be necessary under an operation. For example, a patient is briefly ventilated using up to 100% oxygen during initiation of anesthesia.

The oxygen introduction via the O2 flush 10 can also be regulated such that the oxygen concentration of the breathing gas mixture 5 is, for example, at least 60% at least for a defined short time span. An oxygen saturation of 60% or more, preferably 80% or more, particularly preferably at least 90%, can be used to flush out anesthetics. Under an operation, it can occur that the anesthetics have to be removed quickly from the body of the patient, for example in the event of an allergic reaction of the patient to the anesthetics, for example upon the occurrence of a malign hyperthermia. The rapid removal of anesthetics from the lungs of the patient can be carried out via flooding using pure or highly concentrated oxygen. Such an oxygen flooding of the lungs of the patient can rapidly reduce the anesthetic concentration.

The O2 flush supply line 11 can open into the inspiratory branch 1 of the breathing gas line 4. The oxygen can be introduced upstream before the fan 3 or downstream after the fan 3. In preferred embodiments according to the figures, the oxygen can be introduced downstream after the fan 3 into the breathing gas mixture 5. The O2 flush supply line 11 then opens into the breathing gas line 4 downstream after the fan 3. This offers the advantage that the breathing gas mixture 5 enriched with oxygen does not have to be conducted through the fan 3, which offers enhanced safety. An excessively high oxygen concentration in the fan 3 can result in complications such as a short-circuit, fire, or damage to individual components of the fan 3. In some embodiments, multiple O2 flush supply lines 11 can be comprised, which can open at different points into the breathing gas line 4.

In some embodiments, oxygen can be introduced into the breathing gas line 4 at more than one point via the O2 flush 10, for example, at at least two points. For example, the device 100 can be configured such that the O2 flush 10 can be connected via at least two O2 flush supply lines 11i, 11ii to the breathing gas line 4.

In preferred embodiments according to the figures, the O2 flush can be introduced upstream before the fan 3 and/or downstream after the fan 3 into the breathing gas mixture 5. The at least one O2 flush supply line 11 then opens upstream before the fan 3 and/or downstream after the fan 3 into the breathing gas line 4.

In some embodiments, the oxygen of the O2 flush 10 can be introduced at more than one point into the breathing gas line 4, for example at at least two points. For example, the device 100 can be configured such that the O2 flush 10 can be connected via at least two O2 flush supply lines 11i, 11ii to the breathing gas line 4.

In the specific embodiment according to FIG. 1, the O2 flush supply line 11 can divide into at least two O2 flush supply lines 11i, 11ii, namely, for example, into a first O2 flush supply line 11i and a second O2 flush supply line 11ii.

The first O2 flush supply line 11i and the second O2 flush supply line 11ii can be pneumatically connected to the breathing gas line 4. The first O2 flush supply line 11i and the second O2 flush supply line 11ii can preferably open at different points into the breathing gas line 4.

In some embodiments, the O2 flush supply lines 11i, 11ii can each introduce oxygen simultaneously into the breathing gas line 4 (not shown).

In preferred embodiments, the O2 flush supply lines 11, 11i, 11ii can be regulated via at least one O2 flush switching valve 31 such that in each case only one of the O2 flush supply lines 11i or 11ii can introduce oxygen into the breathing gas line 4. It can be regulated via the O2 flush switching valves 31 via which of the O2 flush supply lines 11, 11i, 11ii oxygen is introduced into the breathing gas line 4. The location of the feed of oxygen into the breathing gas line 4 can therefore be regulated via the O2 flush switching valves 31.

The O2 flush switching valve 31 can be designed as a monostable or as a bistable way valve. In preferred embodiments, the O2 flush switching valve 31 is designed, like the switching valve 32, as a monostable way valve having solenoid and spring. Energizing the solenoid guides the valve 31 actively into a first switching position. The solenoid is inactive when deenergized, so that the spring relaxes and brings the valve into the second switching position. When deenergized, the valve 31 is in its base position.

The O2 flush switching valves 31 can be electronically and/or mechanically switched. The O2 flush switching valves 31 can be switched automatically by the control unit 101 and/or manually by the user. In particular, the O2 flush switching valves 31 can be switched manually so that a user (trained medical personnel) can supply oxygen as needed and can also define the location of the feed into the breathing gas line 4.

It is apparent from FIG. 1 that oxygen is introduced via the first O2 flush supply line 11i when the valve 31 is in its base position, thus is deenergized. The second O2 flush supply line 11ii is supplied when the valve 32 is energized.

In the exemplary embodiment according to FIG. 1, the O2 flush 10 is connected to the O2 flush supply line 11. The O2 flush supply line 11 is connected to the O2 flush switching valve 31. The O2 flush switching valve 31 can be designed, for example, as a 2/3-way valve and can therefore have three fittings and two switching positions. The O2 flush switching valve 31 can then moreover be connected to the first O2 flush supply line 11i and to the second O2 flush supply line 11ii and depending on the switching position can either supply the first O2 flush supply line 11i or the second O2 flush supply line 11ii with oxygen from the O2 flush 10.

In the exemplary embodiment according to FIG. 1, the first O2 flush supply line 11i and the second O2 flush supply line 11ii open at different points into the breathing gas line 4. The device 100 can therefore be configured to regulate the location of the oxygen introduction into the breathing gas line 4.

For example, the first O2 flush supply line 11i can open in the flow direction after the fan 3 into the breathing gas line 4. In some exemplary embodiments it can be particularly advantageous if the O2 flush supply line 11 opens as close to the patient as possible into the breathing gas line 4.

For example, the second O2 flush supply line 11ii can open in the flow direction before the fan 3 into the breathing gas line 4. For example, the second O2 flush supply line 11ii can open in the flow direction before the first check valve 21 into the breathing gas line 4 (FIG. 1).

In alternative embodiments, it is also conceivable that the device 100 comprises, instead of the 2/3-way valve 31, at least two 2/2-way valves 31 (not shown). For example, the first O2 flush supply line 11i can comprise a first O2 flush switching valve, which can be designed as a 2/2-way valve having two fittings and two switching positions. For example, the second O2 flush supply line 11ii can comprise a second O2 flush switching valve, which can likewise be designed as a 2/2-way valve having two fittings and two switching positions.

The two 2/2-way valves can then be switched in parallel by the control unit 101 such that either the first O2 flush supply line 11i or the second O2 flush supply line 11ii can be opened in order to introduce oxygen of the O2 flush into the breathing gas mixture 5.

The device 100 can optionally comprise a separate nitrogen oxide module 43 and at least one nitrogen oxide supply line 44 (not shown). Nitrogen oxides, for example nitrous oxide N2O, can be used, for example, to strengthen the anesthesia and/or alleviate pain. Nitrous oxide N2O can therefore be administered in addition to the at least one volatile anesthetic. The nitrogen oxide module 43 can be configured and arranged in the device in parallel and equivalent to the fresh gas module 6 and/or the oxygen module 10. The nitrogen oxide module 43 can have a separate nitrogen oxide supply line 44. In some embodiments, the nitrogen oxide (nitrous oxide) can also be introduced together with the fresh gas and/or the other anesthetic into the breathing gas line 4. The nitrogen oxide supply line 44 can have a further safety valve in order to monitor the nitrogen oxide concentration. In case of a failure of the oxygen module 10 and/or the fresh gas module 6, the nitrogen oxide supply can be suppressed quickly and reliably by way of the further safety valve.

The supply lines, in particular the fresh gas supply line 7, the O2 flush supply line 11, and the nitrogen oxide supply line 44, can each comprise at least one element in order to ensure the functionality and the safety of the supply lines 7, 11, 44. The elements can be comprised once or multiple times and can vary in their arrangement. The supply lines 7, 11, 44 can be constructed identically or can vary from one another. In particular, the fresh gas supply lines 7, the O2 flush supply lines 11, and the nitrogen oxide supply lines 44 can in principle be constructed nearly identically.

FIG. 2 schematically shows an exemplary structure of the supply lines 7, 11, 44, 71.

The oxygen and/or the oxygen-containing gas mixture and/or the fresh gas and/or the nitrogen oxide can each be acquired from at least one supply source 89. Preferably, at least two supply sources 89 are comprised in each case for reasons of safety. For example, the oxygen and/or the oxygen-containing gas mixture and/or the fresh gas and/or the nitrogen oxide can each be acquired from at least one pressurized gas cylinder 89 and/or from a gas system, for example a central gas system CGS of a hospital, and can each be introduced via the supply lines 7, 11, 44 into the breathing gas line 4.

Fresh gas, oxygen, and nitrogen oxide can in advantageous exemplary embodiments be acquired both from a CGS and also from pressurized cylinders 89. In this case, the device 100 comprises at least two supply lines 7, 11, 44. Oxygen can therefore be fed via a first supply line 11 from a CGS and optionally via a second supply line 11 from a pressurized gas cylinder 89 into the breathing gas line 4. Correspondingly, fresh gas can be fed via a first supply line 7 from a CGS and optionally via a second supply line 7 from a pressurized gas cylinder 89 into the breathing gas line 4. Correspondingly, nitrogen can be fed via a first supply line 44 from a CGS and optionally via a second supply line 44 from a pressurized gas cylinder 89 into the breathing gas line 4. In this case, the control unit 101 can control the supply lines 7, 11, 44 such that the respective feed from the CGS is prioritized. A gas feed from the pressurized gas cylinders 89 is first activated if the CGS fails or should or can no longer be used for other reasons. The pressurized gas cylinders 89 can therefore be used, for example, as a reserve for an emergency situation. The arrangement of multiple pressurized gas cylinders 89 is likewise conceivable.

At least one filter 82 can be arranged in each of the supply lines 7, 11, 44. The filters 82 are configured and designed to filter out possible solid particles from the introduced fluid. The filters 82 are configured as an optional safety element. The filters 82 can preferably be arranged downstream directly after the supply sources 89. Optionally and additionally, moreover water separators and/or oil separators can be arranged (not shown) in or at the supply lines 7, 11, 44.

At least one sensor 84, 85 can be arranged in or at each of the supply lines 7, 11, 44. The sensors 84, 85 can be configured and designed to detect or monitor the pressure and/or the volume and/or the flow in the supply lines 7, 11, 44. For example, at least one pressure sensor 84 can be arranged in each of the supply lines 7, 11, 44. The pressure sensors 84 can detect how much pressure and/or volume is withdrawn from the pressurized gas cylinders 89. The fill level of the pressurized gas cylinders 89 can be monitored and transmitted to the control unit 101 via the pressure sensors 84. If the fill level falls below a critical level, an alarm can be triggered. For this purpose, the device can comprise an alarm generator which is controlled by the control unit 101 (not shown).

The pressure sensors 84 can preferably be arranged downstream directly after the pressurized gas cylinders 89 and the filter 82. For example, at least one flow sensor 85 can be arranged in each of the supply lines 7, 11, 44. The flow sensors 85 can detect the gas flow in the supply lines 7, 11, 44. The flow in the supply lines 7, 11, 44 can be monitored via the flow sensors 85 and transmitted to the control unit 101. The flow sensors 85 can preferably be arranged downstream directly before the opening into the breathing gas line 4. A fine regulation of the flow can be implemented with the aid of the flow sensors 85.

At least one pressure regulator 80 and/or a stenosis 81 can be arranged in or on each of the supply lines 7, 11, 44. The pressure regulator 80 can be designed as a pressure reducer 80. The pressure reducer 80 and/or the stenosis 81 can be configured to regulate the pressure from the supply sources 89 such that the pressure is safely below the lowest specified supply pressure. The supply pressures of the central gas supplies and the pressurized gas cylinders can vary strongly. The dosing valves for the gases require a constant pilot pressure, however, since their characteristic curve depends thereon. Therefore, the pressure regulators 80 and/or the stenoses 81 regulate the pressures to a value which is safely below the lowest specified supply pressure. The dosing valves can thus always operate identically.

There can be pressures up to approximately 20 MPa in the pressurized gas cylinders 89. The pressure reducers 80 and/or the stenoses 81 are configured and designed to regulate the pressure from the pressurized gas cylinders 89 to at least half, preferably at least one-fourth, particularly preferably at least one-tenth of the pressure originally prevailing in the pressurized gas cylinders 89. For example, the pressure reducers 80 and/or the stenoses 81 are configured and designed to regulate the pressure from approximately 20 MPa, preferably to typically 450+/−50 kPa. The pressure reducers 80 and/or the stenoses 81 can be configured and designed to set the pressure such that it is less than the lowest operating pressure of the device 100.

At least one valve 83, 86, 87, 88, 31, 32 can be arranged in or at each of the supply lines 7, 11, 44. The supply lines 7, 11, 44 preferably each comprise at least one overpressure valve 83, preferably at least two overpressure valves 83. The overpressure valves 83 can be configured as opening pressure valves and can be designed to discharge the corresponding gas from the supply line 7, 11, 44 in the event of excessively high pressures in the supply lines 7, 11, 44.

A first overpressure valve 83 can be arranged downstream after the pressure regulator 80 and/or the stenosis 81. The first overpressure valve 83 can be configured and designed to discharge pressure. This offers special protection for the case that the pressure regulator 80 fails and an excessively high pressure from the supply source 89 prevails in the supply lines 7, 11, 44.

In some embodiments, at least one second overpressure valve 83 can be arranged in the supply lines 7, 11, 44. For example, the second overpressure valve 83 can be arranged upstream directly before the introduction into the breathing gas line 4. The overpressure valves 83 offer the advantage of an additional safeguard. Excess gas can be discharged into the surroundings. In some embodiments, the excess gas can also be deliberately discharged and reused (not shown).

Overpressure valve 83 and stenosis 81 are configured and designed to limit the maximum flows. In particular, overpressure valve 83 and stenosis 81 can limit the maximum flows in case of fault. In case of a defect of the pressure regulator 80, thus for the case that the pressure regulator 80 lets through all gas from the supply sources 89, the overpressure valve 83 can discharge the excess gas. Upon failure of the pressure regulator 80, the stenoses 81 can be configured to limit the maximum flow such that the overpressure valve 83 is protected and the overpressure valve 83 can discharge the flow.

Moreover, the supply lines 7, 11, 44 preferably each comprise at least one check valve 86. In some embodiments, the check valve 86 can be designed as a simple check valve. In preferred embodiments, the check valve is designed as a spring-loaded check valve 86. The check valve 86 is configured to supervise the flow direction in the supply lines 7, 11, 44 and in particular to prevent a back flow of gas into the supply sources 89. It is ensured via the check valve 86 that the supply sources 89 are not contaminated by the breathing gas mixture 5 from the breathing gas line 4.

The supply lines 7, 11, 44, 71 can moreover each comprise at least one switching valve 87, 31, 32 and/or at least one dosing valve 88, 88i. Switching valves 87, 31, 32 and/or dosing valves 88, 88i can be designed in preferred exemplary embodiments as bistable valves. The dosing valves 88, 88i can be designed, for example, as needle valves.

The bistable switching valves 87 and/or dosing valves 88 are preferably arranged before the opening into the breathing gas line 4 and are configured to control the supply and/or the supply amount into the breathing gas line 4. For example, the bistable switching valves 87 and/or dosing valves 88 can supervise the flow and/or volume and/or pressure introduced into the breathing gas line 4. In some embodiments, in particular the flow can be supervised via the bistable switching valves 87 and/or dosing valves 88.

For example, at least one switching valve 87 can be arranged in each of the supply lines 7, 11, 44. The, for example, at least one switching valve 87 in each of the supply lines 7, 11, 44 can be designed, for example, as a bistable valve.

For example, at least one bistable switching valve 87 can be arranged in the fresh gas supply lines 7. Moreover, at least one bistable switching valve 87 can be arranged in the nitrogen oxide supply lines 44. Moreover, at least one bistable switching valve 87 can be arranged in the O2 flush supply lines 11. Preferably, all supply lines 7, 11, 44 have at least one bistable switching valve 87. The bistable switching valves 87 can each be arranged upstream directly after the check valves 86.

The bistable switching valves 87 can preferably be switched into an open position or into a closed position. An open position releases the gas through flow unrestrictedly. A closed position can hermetically seal the supply line and prevent the gas flow into the supply line. Preferably, the bistable switching valves 87 can be adjusted from an open position into a closed position. The position of the bistable switching valves 87 can be controlled by the control unit 101.

It can be regulated via the position of the switching valves 87 which of the lines 7, 11, 44 is open and which is closed. It can therefore be regulated via the position of the switching valves 87 from which supply sources gas is conducted into the breathing gas line 4. For example, it can be regulated via the position of the switching valves 87 whether the respective gas is introduced from the CGS or from the pressurized gas cylinders or not at all into the breathing gas line 4.

The bistable switching valves 87 can be configured such that power is required for the change between an open and a closed state and vice versa. This switching energy can be selected to be very low here. The bistable switching valves 87 then remain open or closed without additional energy being required. The bistable switching valves 87 can therefore remain without power in the predetermined position. This offers the advantage that in an emergency situation, for example in the event of a power failure or low-power availability or a software crash or another technical fault, the last selected functions and settings are maintained and ventilation is still possible. The bistable switching valves 87 can moreover be configured here so that manual switching is possible. The medical personnel therefore remain capable of action in an emergency situation.

The device 100 offers the possibility of manual ventilation to preserve the safety for the patients. In the event of power and/or software failure, the device 100 switches to a state, without action of the user, in which the user can still supply the patient with the manual ventilation in an emergency. Pressure changes have to be possible here. The fresh gas has to continue to flow, the CO2 has to be absorbed and/or diffuse away. For this reason, all valves in the software module 10 and/or fresh gas module 6 remain in their last switching position in the event of power or software failure.

In some embodiments, the bistable switching valves 87 can also be configured and designed such that the closed position is only maintained with energy consumption. Therefore, gas can only be introduced into the breathing gas line 4 when the bistable switching valves 87 are deenergized. This offers the advantage that the gas introduction into the breathing gas line 4 is ensured in an emergency situation, for example upon power failure. It can therefore be ensured that the oxygen and/or fresh gas supply can remain. Moreover, it can be ensured that the anesthetic gas supply can remain and the anesthesia is maintained.

In some embodiments, the bistable switching valves 87 can also be configured and designed such that the open position is only maintained with energy consumption. Therefore, gas can only be introduced into the breathing gas line 4 when the bistable switching valves 87 are supplied with power. This can offer the advantage that the gas introduction into the breathing gas line 4 is interrupted in an emergency situation, for example upon power failure, and manual ventilation is enabled, for example ventilation using a manual bag.

Alternatively or additionally, at least one dosing valve 88, 88i can be arranged in each of the supply lines 7, 11, 44, 71. The respective at least one dosing valve 88, 88i in the supply lines 7, 11, 44, 71 can be designed, for example, as a needle valve 88, 88i.

In alternative embodiments, the dosing valve 88 can also be designed as a proportional valve. In some embodiments, the dosing can also take place via the arrangement of multiple switching valves having different apertures, so that the flow is settable via different aperture sizes/powers.

In preferred embodiments, however, the dosing valve 88 is designed as a needle valve. Needle valves offer the advantage that nearly infinitely many switching positions can be implemented. Very exact dosing is possible via needle valves. Needle valves offer the advantage of a very large control range, in which both very small flow amounts and also very large flow amounts are exactly settable.

For example, at least one needle valve 88 can be arranged in the fresh gas supply lines 7. Moreover, at least one needle valve 88 can be arranged in the nitrogen oxide supply lines 44. Moreover, at least one needle valve 88 can be arranged in the O2 flush supply lines 11. Preferably, all supply lines 7, 11, 44 each have at least one needle valve 88.

Needle valves 88 offer the advantage that a very fine pressure regulation is enabled with small flows. At the same time, needle valves 88 can permit very large flows. The needle valves 88 can be configured and designed such that the flow can be set both very small (0-30 l/min) and also very large (80-120 l/min) and also in any moderate level. The device is therefore suitable both for ventilation under anesthesia, in which rather smaller flows are required, and also for ventilation using large flows, for example high flow ventilation.

For the same reason, the device 100 is in particular suitable both for the ventilation/anesthesia of adults, who require larger flows, and also for the ventilation/anesthesia of children, toddlers, newborns, and premature infants, who require smaller flows.

The needle valve 88 can be configured and designed such that a flow of 0 to 100 l/min is introduced into the breathing gas line 4. Preferably, a flow of 0 to 60 l/min is introduced into the breathing gas line 4.

The device 100 can be operated in different operating modes, for which different flow rates can be set. The flow rate is generally set automatically via the control unit 101. The flow rate can be set, for example, via the settings of the needle valves 88 in the supply lines 7, 11, 44.

The needle valves 88 can be configured and designed such that a flow of 0 to 120 l/min can be generated. In a specific exemplary embodiment, the needle valves 88 can be configured and designed such that a flow of 0 to 60 l/min can be generated.

In an operating mode for a high flow ventilation, a flow of up to 120 l/min can be generated, for example of up to 100 l/min. for a high flow therapy, the flow can be conducted through the inactivated fan 3, so that the flow can be generated solely by the oxygen module 10 and/or the O2 flush supply line 11.

In an operating mode for a total intravenous anesthesia, a flow of less than 60 l/min can be generated, for example of less than 30 l/min, preferably less than 20 l/min. For example, in an operating mode for a total intravenous anesthesia, a flow of 18 l/min is generated.

In an anesthesia mode using volatile anesthetics VA, a flow of less than 60 l/min can be generated, for example of less than 30 l/min, preferably less than 20 l/min. For example, in an anesthesia mode using volatile anesthetics VA, a flow of 18 l/min is generated.

In an operating mode for a CPAP ventilation, a flow of less than 60 l/min can be generated, for example of less than 30 l/min, preferably less than 20 l/min. For example, in a CPAP mode, a flow of 15 l/min is generated.

In some embodiments, the needle valves 88 can be designed as bistable needle valves having stepping motors. The needle valves 88 can thus function as dosing valves. A combination of the needle valves with stepping motors offers the advantage that very small and also very large flows can thus be dosed. Needle valves having stepping motors also have very good resolution with very small flows.

The bistable needle valves 88 having stepping motors can be configured and designed so that they remain in the respective last set position without energy supply. This offers the device 100 a special safety function in the event of a power failure, since the fresh gas and/or oxygen and/or nitrogen oxide feed can then remain.

It is advantageous if the device 100 has at least one accumulator. The needle valves 88 can then be adjusted via the energy of the accumulators in the event of a power failure of the power grid. The needle valves 88 can moreover be configured and designed such that a manual adjustment is possible in the event of a power failure. The trained medical personnel are therefore also capable of action in emergency situations.

The supply lines 7, 11, 44 can moreover each comprise at least the switching valve 31, 32, which was already described above herein for FIG. 1. The switching valves 31, 32 are preferably arranged directly before the opening of the supply line into the breathing gas line 4. The point of the breathing gas line 4 at which the corresponding gas is introduced is controlled via the switching valves 31, 32. The switching valves 31, 32 are configured and designed to regulate the feed point of the gases (fresh gas, oxygen).

The device 100 can optionally comprise at least one anesthetic module 8 and at least one anesthetic supply line 9. The anesthetic module 8 is configured and designed to provide anesthetics to the system via the anesthetic supply line 9. The anesthetic module 8 can be actuated via the control unit 101.

The device 100 can be used for inhalation anesthesia or inhalation narcosis. Volatile anesthetics VA can be used for inhalation anesthesia or inhalation narcosis. For example, the volatile anesthetics VA can be selected from the following group: isoflurane, sevoflurane, desflurane, halothane, enflurane, methoxyflurane. Particularly preferred volatile anesthetics VA are selected from the following group: isoflurane, sevoflurane, desflurane, halothane.

For inhalation anesthesia or inhalation narcosis, alternatively or additionally gaseous anesthetics can also be used, such as xenon, argon, or dinitrogen monoxide N2O (nitrous oxide). In particular nitrous oxide can be added in some applications to the breathing gas mixture 5 in order to assist or positively influence the efficacy of the volatile anesthetics VA.

The volatile anesthetics VA are used to create optimum conditions for the patient with respect to at least the following parameters
unconsciousness (patient sleeps/hypnosis)
freedom from pain/pain perception (analgesia)
relaxation of the muscle tension
damping of vegetative reflexes and of defensive reflexes

The volatile anesthetics VA have low molecular weight and have a high vapor pressure and a relatively low boiling point.

Overview of volatile anesthetics VA

| | | | | Vapor | | | Setting |
|---|---|---|---|---|---|---|---|
| | | Molar | Boiling | pressure | MAC - | MAC - | range upon |
| | | mass | point | (ambient | Awake | Immobile | introduction |
| Name | Structure | [g] | [° C.] | 20° C.) | [%] | [%] | max. [%] |
| isoflurane | CF3—CHCl—O—CHF2 | 185 | 48.5 | 313 hPa | 0.43 | 1.28 | 5 |
| | | | | (31.3%) | | | |
| sevoflurane | CH—CF3—CF3—O—CH2F | 200 | 58.6 | 207 hPa | 0.63 | 2.05 | 8 |
| | | | | (20.7%) | | | |
| desflurane | CF3—CHF—O—CHF2 | 168 | 22.8 | 873 hPa | 2.40 | 6.00 | 18 |
| | | | | (87.3%) | | | |
| halothane | CF3—CHBrCl | 197 | 50.2 | 242 hPa | 0.52 | 0.75 | 5 |
| | | | | (24.2%) | | | |
| enflurane | CHF2—O—CF2—CHFCl | 184 | 56.5 | 233 hPa | — | 1.58 | 5 |
| | | | | (23.3%) | | | |
| methoxyflurane | CH3—O—CF2—CHCl2 | 165 | 104.7 | 26 hPa | — | 0.2 | 4 |
| | | | | (2.6%) | | | |

MAC = minimum alveolar concentration
1 MAC = 50% of the patients are ideally anesthetized

In some embodiments, the anesthetics can be introduced in gaseous form into the breathing gas line 4 (not shown). For a gaseous introduction, the anesthetics VA or the gaseous anesthetics can be mixed beforehand with fresh gas. For this purpose, the device 100 can have a mixing chamber (not shown). The gaseous anesthetics mixed with fresh gas can then be conducted via the anesthetic supply line 9 into the breathing gas line 4, where they mix with the breathing gas mixture 4.

In preferred embodiments, the volatile anesthetics VA are introduced via liquid dosing into the breathing gas line 4. For this purpose, the device 100 can have at least one vaporization element 8a. The vaporization element 8a can be arranged in or at the breathing gas line 4. The vaporization element 8a can be formed as an integral component of the breathing gas line 4. The volatile anesthetics VA can then be conducted via the anesthetic module 8 and the anesthetic supply line 9 into the vaporization element 8a of the breathing gas line 4. The anesthetic module 8 is pneumatically connected for this purpose via the anesthetic supply line 9 to the vaporization element 8a of the breathing gas line 4. The volatile anesthetics VA introduced in liquid form can vaporize in the vaporization element 8a and combine with the breathing gas mixture 5.

A liquid dosing of the volatile anesthetics VA can be implemented in that the anesthetics VA are cooled in a tank and/or placed under pressure. In preferred embodiments, a first pressure P1 is applied in the breathing gas line 4 and a second pressure P2 is applied at least in the anesthetic supply line 9, wherein the first pressure P1 is less than the second pressure P2. The second pressure P2 is, for example, at least 100 kPa. In preferred exemplary embodiments, the second pressure P2 can be at least 180 kPa. The volatile anesthetics VA can therefore be introduced via the anesthetic module 8 and the anesthetic supply line 9 in liquid form into the vaporization element 8a. The anesthetics VA can first vaporize therein and combine with the breathing gas mixture 5.

The concentration of anesthetics VA is generally selected such that the anesthetic concentration in the breathing gas mixture 5 is, for example, in a range between 0 and 25%. The device 100 is configured such that a user, for example trained medical personnel such as the anesthetist, can set the concentration of the anesthetics VA very accurately and variably. The anesthetist can set the concentration of the anesthetics depending on the anesthetic used and depending on the patient and/or type of the operation and can adjust adaptively to the conditions under the operation.

For a sedation of the patient 90, the concentration of the individual volatile anesthetics VA in the breathing gas mixture 5 is typically selected as follows: isoflurane 0 to 5%; halothane 0 to 5%; sevoflurane 0 to 8%; desflurane 0 to 18%. The volatile anesthetics VA are generally not used together. Higher concentrations are possible and are at the discretion of the user, thus in particular at the discretion of the anesthetist. The device 100 is configured and designed to permit the setting of all concentrations.

The liquid dosing of the volatile anesthetics offers the following advantages over the gas dosing with prior mixing with fresh gas, for example:
more exact and therefore more frugal dosing of anesthetics is possible
the supply of anesthetics is independent of the supply of fresh gas or oxygen
the anesthetic concentration is independent of the fresh gas concentration and/or the oxygen concentration
the pressure from anesthetic pressurized gas cylinders can supply the conveying energy for the anesthetics

The anesthetic supply line 9 can open into the vaporization element 8a. The physical state of the volatile anesthetics VA changes in the vaporization element 8a. Upon entry into the vaporization element 8a of the breathing gas line 4, the volatile anesthetics VA are subjected to the pressure P1 of the breathing gas line 4 and thus become gaseous. The vaporization element 8a can be arranged in the inspiratory branch 1 of the breathing gas line 4. The anesthetics VA can therefore be introduced into the inspiratory branch 1 of the breathing gas line 4. The vaporization element 8a can be arranged upstream before the fan 3 or downstream after the fan 3. In preferred embodiments according to the figures, the vaporization element 8a can be arranged upstream before the fan 3. The vaporization element 8a can preferably be arranged downstream after the fresh gas supply lines 7i, 7ii. In the specific exemplary embodiments according to the figures, the vaporization element 8a is arranged between the opening of the first fresh gas supply line 7i into the breathing gas line 4 and the fan 3.

The device 100 is advantageously configured such that at least one permanent flow, the byflow, is formed in the breathing gas line 4. The byflow ensures that the breathing gas mixture 5 in the breathing gas line 4 is always in motion. The byflow has positive effects on the vaporization rate of the volatile anesthetics VA in the vaporization element 8a.

The byflow in the vaporization element 8a can be generated and maintained by the reservoir 12 and/or by the fan 3 and/or by the fresh gas module 6 and/or by the oxygen module 10. The volatile anesthetics VA fed in liquid form can vaporize due to the byflow in the vaporization element 8a and mix optimally with the breathing gas mixture 5.

FIG. 3 schematically shows a portion of the breathing gas line 4, in which the vaporization element 8a is arranged, which is connected via the anesthetic supply line 9 to the anesthetic module 8.

It is apparent from FIG. 3 that the vaporization element 8a can be arranged in the breathing gas line 4 between the first check valve 21 and the fan 3. The vaporization element 8a can preferably be arranged upstream directly before the fan 3. The vaporization element 8a can preferably be arranged downstream after the fresh gas feed point 207 of the first fresh gas supply line 7i. The vaporization element 8a can preferably be arranged downstream after the first check valve 21. The check valve 21 can then prevent the breathing gas mixture 5 conveyed in the breathing gas line 4 from flowing against the main flow direction 5.

The anesthetic module 8 is connectable via the anesthetic supply line 9 to the vaporization element 8a. An anesthetic feed 209 into the breathing gas mixture 5 can take place via the vaporization element 8a. Different volatile anesthetics VA can be supplied to the breathing gas mixture 5 via the anesthetic module 8. In general, only one anesthetic at a time is added in each case to the patient. It is also possible via the anesthetic module 8 to exchange the respective anesthetic under the operation. At least one volatile anesthetic VA can be introduced in liquid form into the vaporization element 8a via the anesthetic module 8. The volatile anesthetic VA can vaporize in the vaporization element 8a and mix with the breathing gas mixture 5.

To provide the volatile anesthetics VA in liquid form, the anesthetic module 8 and the anesthetic supply line 9 can be cooled and/or kept under pressure. Anesthetic module 8 and anesthetic supply line 9 are preferably kept under the second pressure P2. The pressure P2 is preferably at least 180 kPa. At this pressure, the volatile anesthetics VA are in the liquid physical state, as long as the temperature is below 40° C. Preferred volatile anesthetics VA are selected, for example, from the following group: isoflurane, sevoflurane, desflurane, halothane.

The anesthetic module 8 can comprise at least one of the following units: pressure supply unit 810, receptacle unit 820, selection unit 830, dosing unit 840, safety unit 850, temperature unit 860. The anesthetic module 8 is to be understood as a pneumatic unit. All units of the module 8 are pneumatically connected (directly or indirectly) to one another. The connection can exist via the at least one anesthetic supply line 9. The anesthetic module 8 and the anesthetic supply line 9 are preferably under the pressure P2 in order to keep the volatile anesthetic VA to be conveyed in liquid form. The storage and/or dispensing and/or supervision of anesthetics from the anesthetic module 8 can be controlled by the control unit 101.

The anesthetic module 8 can be connectable to at least one device for storing volatile anesthetics 800. The devices for storing volatile anesthetics can be designed as anesthetic tanks 800. The at least one tank 800 is configured and designed to accommodate and/or store and/or dispense volatile anesthetics VA. In this case, an individual tank 800 is preferably used in each case for a single volatile anesthetic VA.

The tanks 800 are configured and designed to each accommodate and/or store and/or dispense a volatile anesthetic VA. In this case, the tanks 800 are preferably under pressure such that the stored volatile anesthetics are present in a liquid physical state at room temperature. The pressure in the tank 800 can be in a range between 100 kPa and 500 kPa, in order to keep the anesthetics liquid. The pressure in the tanks 800 is preferably in a range between 150 kPa and 300 kPa. The pressure in the tanks 800 is, for example, at least 180 kPa. The tanks 800 are therefore also configured and designed to accommodate and/or retain and/or dispense a pressure.

The tanks 800 can be refillable with anesthetics VA. The tanks 800 can be configured and designed such that the fill level of the anesthetic is detectable (not shown). The detection of the fill level can take place visually. For this purpose, the tanks 800 can be at least partially transparent. For example, the tanks 800 can have a viewing window, optionally having a scale, through which the filling of the tanks 800 can be visually read. The detection of the fill level can also be carried out by a sensor. For this purpose, the tanks 800 can comprise a float element which floats on the anesthetic and is magnetic, for example. The field strength can be detected via a magnetic sensor. It can be determined on the basis of the field strength where the float element is located. The fill level of the anesthetic can then be determined on the basis of the location of the float element.

The tanks 800 can moreover have elements which permit a unique assignment (not shown). For example, a code can be stored at or in the tanks 800, which permits a unique assignment of the individual tank 800 or the anesthetic contained therein. The code can be produced, for example, mechanically and/or sensorially and/or visually.

To connect the tanks 800 to the device 100, the anesthetic module 8 can comprise the at least one receptacle unit 820. The receptacle unit 820 comprises at least one receptacle bay 821. The receptacle unit 820 can accommodate at least one tank 800 via the receptacle bays 821. The receptacle unit 820 preferably comprises more than one receptacle bay 821 for accommodating multiple different tanks 800, for example two (see FIG. 3) or more (not shown).

The device 100 can have a special receptacle bay 821 for each individual tank 800, thus for each anesthetic. In some embodiments, it is also conceivable that a receptacle bay 821 is designed to accommodate different individual tanks 800 having different anesthetics.

The receptacle bays 821 can have at least one device (not shown here) for recognizing the individual tanks 800. This offers an additional safety aspect, since therefore confusing the different anesthetics can be precluded. The recognition can take place mechanically and/or visually and/or sensorially. It can thus be ensured that the correct tank 800 having the correct anesthetic is accommodated in each case in the receptacle bay 821 intended for it. The receptacle bays 821 can have, for example, tank-specific connection devices for mechanical recognition of the individual tank 800. The recognition can alternatively or additionally also be visually coded, for example via a color coding of the individual tanks 800 and their respective receptacle bay 821. The recognition can alternatively or additionally also be carried out in a sensorially or electrically coded manner. For this purpose, the tanks 800 can have, for example, a barcode or the like and the receptacle bays 821 can have a corresponding sensor. The receptacle unit 820 is configured and designed to accommodate one or more tanks 800 such that the tanks 800 are under pressure. The receptacle unit 820 is preferably configured to keep the pressure in the tanks 800 at, for example, at least 180 kPa. For this purpose, the receptacle unit 820 is configured and designed to supply pressure to the tanks 800 and/or discharge pressure from the tanks 800. For this purpose, the receptacle unit 820 can be connected to at least one pressure supply unit 810.

The pressure supply unit 810 is configured and designed to supply the receptacle unit 820 and therefore the tanks 800 with pressure or to relieve the pressure. The pressure supply unit 821 can supply pressure and/or remove pressure. For example, the pressure supply unit 810 can be connected to the fresh gas module 6 and/or the oxygen module 10 in order to acquire fresh gas and/or oxygen to provide a pressure. In one preferred embodiment, the pressure supply unit 810 acquires fresh gas having a regulated pilot pressure in order to provide the pressure.

The pressure supply unit 810 can preferably comprise a pressure regulator to regulate the pressure from the fresh gas module 6 to the pressure level required in the tanks 800. The pressure supply unit 810 can moreover comprise at least one pressure sensor to detect the pressure in the respective tanks 800. The pressure supply unit 810 can moreover comprise at least one temperature sensor to detect the temperature. The device 100 can comprise at least one alarm generator (not shown). The anesthetic module 8 can interact with the alarm generator via the control unit 101. The alarm generator can thus output an alarm, for example, if the temperature increases above the value at which it cannot be ensured that all volatile anesthetics can still be kept liquid. The alarm generator can alternatively or additionally also output an alarm if the pressure falls below a value at which it cannot be ensured that all volatile anesthetics can still be kept liquid.

The pressure supply unit 810 can preferably have its own reservoir for storing fresh gas (not shown), so that the pressure can be maintained for a certain time even without an access to the fresh gas module 6. The pressure supply unit 810 can moreover comprise at least one check valve, so that the fresh gas supply can only run in the direction from the fresh gas module 6 to the pressure supply unit 810 and not vice versa.

The pressure supply unit 810 can comprise at least one valve for loading 811 and at least one valve for relieving 812 the receptacle unit 820 and/or the tanks 800. The receptacle unit 820 and/or the tanks 800 can thus be supplied a pressure via the fresh gas module 6 and the valve 811. Pressure can be discharged from the receptacle unit 820 and/or the tank 800 via the valve 812. For this purpose, the anesthetic module 8 is connected to the outlet 14-A, so that the pressure can be discharged via the valve 812 and the outlet 14-A.

The pressure supply unit 810 can comprise at least one switching valve 813 to select the respective tank 800 for supply. In specific embodiments, a plurality of valves 811, 812, 813 is advantageous, which can be actuated by the control unit 101 or directly by a user such that the pressure in all tanks 800 in use can be regulated.

The receptacle unit 820 is furthermore configured and designed to accommodate one or more tanks 800 such that the tanks 800 can accommodate and/or store and/or dispense anesthetics. The accommodation of the anesthetic in the respective tank 800 can take place inside the receptacle bays 821 or outside. For this purpose, the tanks 800 can each comprise a filling valve (not shown). The accommodation of anesthetics can take place when the tanks 800 are vented. The accommodation of anesthetics can also take place when the tanks 800 are under pressure. This can be advantageous in particular for desflurane, since the vapor pressure at 40° C. nearly reaches the pressure of 1.8 bar.

The receptacle unit 820 is furthermore configured and designed to ensure controlled dispensing of anesthetics from the tanks 800. The anesthetic module 8 is configured and designed such that the dispensing of anesthetics from the tanks 800 into the anesthetic supply line 9 can only take place if the respective tank 800 and the anesthetic supply line 9 are loaded. The dispensing of the volatile anesthetic VA then takes place in liquid form.

The valves of the receptacle unit 820 and/or the tanks 800 and/or the pressure supply unit 810 are configured and designed such that they permit various functions in cooperation, such as:
tanks 800 are inserted into the receptacle bays and are under pressure and supply volatile anesthetics VA
tanks 800 are inserted into the receptacle bays and are under pressure and do not supply volatile anesthetics VA
tanks 800 are inserted into the receptacle bays and are under pressure and can be filled with volatile anesthetics VA
tanks 800 are inserted into the receptacle bays and are not under pressure and can be filled with volatile anesthetics VA
tanks 800 are inserted into the receptacle bays and are not under pressure and can be removed

The receptacle unit 820 can be pneumatically connected to the selection unit 830 via the anesthetic supply line 9. The selection unit 830 of the anesthetic module 8 is configured and designed to select the respective anesthetic. In this case, in general only one volatile anesthetic VA at a time is always used, in order to avoid undesired interactions between the volatile anesthetics VA. The anesthetic is selected by the trained medical personnel. An input can be performed on the device 100 for this purpose.

The selection unit 830 (not shown in more detail here) comprises at least one selection valve 831. The selection valve 831 can be designed, for example, as a 2/2-way switching valve. In a specific exemplary embodiment, the selection unit 830 can comprise multiple selection valves 831, in particular one for each individual anesthetic.

The selection valves 831 are preferably designed as bistable 2/2 way switching valves. The selection valves 831 can each be configured and designed such that they obstruct the path to the dosing unit 840 and/or to the vaporization element 8a in a deenergized state. Energizing can switch one of the selection valves 831 in each case such that the path to the dosing unit 840 and/or to the vaporization element 8a is released. It is preferably stored in this case in the control unit 101 that only one selection valve 831 at a time can be opened in each case. It is thus ensured that only one of the anesthetics is fed into the breathing gas line 4 at a time. Check valves can be arranged behind each of the selection valves 831 to prevent a backflow of anesthetics (not shown).

The selection unit 830 can be pneumatically connected to the dosing unit 840 via the anesthetic supply line 9. The dosing unit 840 of the anesthetic module 8 is configured and designed to dose the respective selected anesthetic.

The dosing unit 840 (not shown in more detail here) can comprise at least one dosing valve 841 for this purpose. The dosing valves 841 are configured and designed to enable dosing of volatile anesthetics. The dosing can take place in a liquid or gaseous physical state of the anesthetics. In preferred exemplary embodiments, the dosing takes place in a liquid physical state of the anesthetics. The dosing valves 841 can be configured and designed to apply a flow and/or a volume and/or a pressure. The dosing valves 841 can preferably specify a specific flow. The dosing valves 841 can preferably be configured and designed to permit continuous dosing. The dosing valves 841 can be designed in some exemplary embodiments as needle valves. The dosing valves 841 can be designed in some exemplary embodiments as needle valves having stepping motors. Needle valves offer the advantage of reliable and exact dosing. Moreover, position coding is possible.

The dosing valves 841 can be designed and configured to dose different anesthetics VA via position coding. For example, more than one anesthetic can be dispensed in dosed form into the circuit, for example more than two, for example at least three different volatile anesthetics VA. For example, isoflurane and/or sevoflurane and/or desflurane can be dosed in a controlled manner into the circuit via position coding of the dosing valves 841.

The dosing valves 841 can be configured and designed such that the valve position without energizing remains at the last set value and/or falls on an open base setting and/or falls on a closed base setting. In preferred embodiments, the dosing valves 841 can be configured and designed such that the valve position without energy supply remains in the respective last set position. The dosing valves 841 can be configured and designed such that they dose the respective last set flow without energy supply. This offers a special safety function to the device 100 in the event of a power failure, since the anesthesia feed can thus remain, on the one hand, in that the dosing valves 841 remain in their position and, on the other hand, the conveying energy of the anesthetics VA comes from the pressurized gas cylinders.

The dosing unit 840 can additionally comprise at least one flow sensor and/or at least one pressure sensor and/or at least one temperature sensor (not shown). Using these sensors, the flow and/or the pressure and/or the temperature can be detected in the dosing unit 840 and supervised by the control unit 101.

The dosing unit 840 can be pneumatically connected via the anesthetic supply line 9 to the safety unit 850. The safety unit 850 of the anesthetic module 8 is configured and designed to supervise the provision of the anesthetic into the vaporization element 8a. The provision of the anesthetic into the vaporization element 8a can be permitted and/or stopped via the safety unit 850. The safety unit 850 can be pneumatically connected via the supply line 9 to the vaporization element 8a. Furthermore, the safety unit 850 can be pneumatically connected to the outlet 14-A (not shown).

The safety unit 850 comprises at least one valve 851, 852 for this purpose. In some exemplary embodiments, the safety unit 850 can comprise at least two valves 851, 852. The valves 851, 852 can be connected in succession, for example. The valves 851, 852 can preferably be designed as 2/2-way switching valves and/or as 3/2-way switching valves. The valves 851, 852 can preferably be designed as monostable.

In a specific exemplary embodiment, the safety unit 850 comprises a first valve 851 and a second valve 852. The valves 851, 852 can be arranged, for example, between the dosing valve 841 and the vaporization element 8a in the anesthetic supply line 9.

The first valve 851 can be designed, for example, as a monostable 2/2 way switching valve and can be configured to allow or close the path to the vaporization element 8a. It can be provided that the valve 851 is closed without power and is open when energized or vice versa. An anesthetic flow into the vaporization element 8a is preferably only possible when the first valve 851 is energized. The first valve 851 can therefore be used as a safety valve so that the introduction of anesthetics into the vaporization element 8a can only take place actively. In an emergency situation (power and/or software failure), the valves 851 can therefore automatically interrupt the introduction of volatile anesthetics. However, the first valve 851 can preferably also be configured so that it can be manually switched. The introduction of volatile anesthetics VA can thus also be established in an emergency situation, since the dosing valves 841 remain in their position and the conveying energy of the anesthetics VA comes from the pressurized gas cylinders and the first valve 851 is open, so that the path into the vaporization element 8a is open.

The second valve 852 can be designed, for example, as a monostable 3/2 way switching valve and can be configured to allow or close the path to the vaporization element 8a and/or to the outlet 14-A. It can be provided that the valve 852 releases the path to the vaporization element 8a without power and releases the path to the outlet 14-A when energized or vice versa. An anesthetic flow into the outlet 14-A is preferably only possible when the second valve 852 is energized. Anesthetic can therefore be actively discharged into the outlet 14-A via the second valve 852. The safety unit 850 can therefore in particular also be configured and designed to flush anesthetics VA out of the anesthetic supply line 9. This can be advantageous in particular upon a change of the anesthetic. The flushing out of the anesthetic supply line 9 can prevent more than one anesthetic at a time from being located in the anesthetic supply line 9.

The safety unit 850 can additionally comprise an inclination sensor (not shown). The spatial orientation of the vaporization element 8a and/or the device 100 as a whole can be ascertained via the inclination sensor.

The temperature unit 860 of the anesthetic module 8 is configured and designed to detect and/or regulate the temperature in or at the vaporization element 8a. The temperature unit 860 can comprise at least one temperature sensor to detect the temperature in or at the vaporization element 8a. Furthermore, the temperature unit 860 can comprise at least one heating element 861. The at least one heating element 861 can be configured and designed to influence the temperature in the vaporization element 8a. For example, the heating elements 861 can heat the vaporization element 8a, preferably to a temperature above the room temperature. For example, temperatures of up to 40° C. can be set in order to increase the vaporization rate of the anesthetic in the vaporization element 8a. The temperature of the vaporization element 8a affects the vaporization rate of the anesthetic. Basically: The higher the temperature, the higher the vaporization rate. The vaporization rate can in this way be up to 2 L per minute.

At the beginning of narcosis, vaporization rates up to 2 l/min can be necessary to initiate the narcosis. With saturated fatty tissue, lower vaporization rates can be necessary to maintain the narcosis. The device 100 can then be set such that the vaporization rate is less than 2 l/min, for example less than 1 l/min or less than 0.5 l/min. With saturated fatty tissue, a vaporization rate of 1 ml/min to 100 ml/min can sometimes be sufficient to maintain the narcosis. The vaporization rate can be set variably via the anesthetic module 8.

The device 100 can comprise a plurality of further functional valves 21, 22, 23, 24, 25, 26, 27, 28, 29, 30, which are described hereinafter. Designations “first”, “second”, etc. which are sometimes used herein serve solely to distinguish the various valves and have no technical importance, in particular no prioritization or the like.

It is apparent from FIG. 1 that the device 100 can comprise at least one pressure control valve 30, which is arranged in the breathing gas line 4. The pressure control valve 30 can be configured to influence or regulate the flow direction of the main flow S. The pressure control valve 30 is configured to permit or regulate variable volume flows.

In some embodiments, the pressure control valve 30 can be designed as a bistable switching valve. The pressure control valve 30 can be switched into an open position or into a closed position or into at least one middle position. An open position releases the gas flow unrestrictedly. A closed position can hermetically seal the supply line and prevent the gas flow into the breathing gas line 4. A middle position can release the gas flow with restrictions.

In preferred embodiments, the pressure control valve 30 can be designed as a proportional valve. For example, the pressure control valve 30 can be designed as a proportional 2/2-way valve (see figures).

The pressure control valve 30 is preferably configured to selectively block the flow in at least one direction. The flow direction of the breathing gas mixture 5 is not reversible due to the arrangement of the pressure control valve 30 in the breathing gas line 4.

The pressure control valve 30 is preferably designed as an adjustable pressure control valve 30 for regulating an expiration pressure Pexsp. The pressure control valve 30 can be designed as a PEEP valve and can be configured to set or maintain at least the positive end-expiratory pressure (PEEP). The pressure control valve 30 can prevent the pressure drop during the exhalation from reaching the ambient air pressure. The pressure control valve 30 represents an adjustable stenosis.

The pressure control valve 30 can be electrically set to a value and/or passively regulated to a preset value. The pressure control valve 30 can be set to a patient-specific PEEP. The patient-specific PEEP can be determined and set beforehand. In healthy adults, the PEEP is generally between 15 and 20 hPa. With previously damaged lungs and/or during the operation, a higher PEEP can be necessary. The PEEP is pre-settable and/or settable during use.

The operating pressure of the pressure control valve 30 can be electrically settable. The operating pressure of the pressure control valve 30 is preferably settable to a PEEP of 0 to 100 hPa, preferably of 0 to 80 hPa, particularly preferably of 3 to 80 hPa. The pressure control valve 30 is configured and designed to adopt arbitrary switch positions in its operating range. The setting of the pressure control valve 30 can be controlled manually and/or by the control unit 101.

The pressure control valve 30 can be configured such that power is only required to change the operating pressure. The pressure control valve 30 can therefore be configured to remain in its preset position without power being required.

In some embodiments, the pressure control valve 30 can be configured such that it returns to a basic state having a defined pressure in the deenergized state. In the deenergized basic state, the PEEP valve 30 can passively regulate, for example, to a range between 3 and 10 hPa. In a specific exemplary embodiment, the PEEP valve 30 can passively regulate, for example, to a PEEP of 5 hPa. The device 100 can therefore maintain a PEEP of, for example, 5 hPa even in an emergency situation, for example in the event of a power failure.

At least the byflow flows permanently through the PEEP valve 30 and seals it off. The byflow has a positive effect on the functionality of the PEEP valve 30.

The device 100 is configured and designed to perform complex maneuvers for determining the optimum ventilation parameters. For example, the PEEP and/or the inspiration pressure can be automatically determined. For example, loops about pressures and/or flows and/or volumes can also be applied, such as pressure-volume loops (P-V loops). The device can therefore advantageously be used for ventilation and anesthesia and nonetheless can perform complex maneuvers such as loops or PEEP finder.

The device 100 can comprise an array of further valves which are used for safety and/or the function of the device. For example, the device 100 can comprise valves 21, 22, 23, 24, 25, 26, 27, 28, 29. The valves 21, 22, 23, 24, 25, 26, 27, 28, 29 can be designed, for example, as check valves. Check valves can supervise and/or restrict and/or prevent the flow of a gas, for example the breathing gas mixture 5, in one direction. Opening the check valve releases the flow. Closing the check valve blocks the flow selectively in at least one direction. Check valves can restrict or prevent a backflow of the gas counter to the flow direction.

The valves 21, 22, 23, 24, 25, 26, 27, 28, 29 can each be designed as a
simple check valve or as a
blockable check valve having a solenoid, for example, or as a
simple loaded check valve having, for example, a spring or as an
adjustable loaded check valve having, for example, a solenoid and a spring or as a
continuously adjustable loaded check valve having, for example, a solenoid, a spring, and a stepping motor.

Simple check valves only release the flow in one direction. In particular the valves 21, 22, 23 can be designed, for example, as simple check valves.

Blockable check valves in the idle position can release the flow in one direction and block it in one direction. Blockable check valves in an active position, for example, upon energizing of the solenoid, can block the flow in both directions. The valve 28 can preferably be designed as a blockable check valve.

Loaded check valves release the flow in only one direction and are closed in an idle position. Simple loaded check valves are loaded using a spring, for example, and open at a predefined pressure (operating pressure) depending on the spring setting. The operating pressure in simple spring-loaded check valves can be uniform. The valve 27 can preferably be designed as a simple loaded check valve.

The operating pressure in adjustable spring-loaded check valves can be adjustable. The adjustment of the operating pressure of loaded check valves and the blocking of blockable check valves can take place automatically and/or manually. The operating pressure of the loaded or blockable check valves can be adjusted, for example, via the spring tension and/or using a solenoid and/or using a stepping motor. Adjustable loaded check valves can also comprise, for example, in addition to the spring, a solenoid, which can at least partially or completely counteract the spring tension. The spring tension is reduced or canceled out entirely upon energizing of the solenoid, so that the flow can be increased or released. The spring is active when deenergized, so that the flow is reduced or closed. The valves 24, 25, 26 can be designed, for example, as adjustable loaded check valves having spring and solenoid. An additionally comprised stepping motor permits a continuous adjustment of the flow. The APL valve 29 can be designed, for example, as a continuously adjustable loaded check valve having spring, solenoid, and stepping motor.

The device 100 can comprise, for example, at least one simple check valve 21, 22. For example, the device 100 can have at least one first simple check valve 21 in the inspiratory branch 1 and additionally or alternatively at least one second simple check valve 22 in the expiratory branch 2.

In the specific exemplary embodiment according to FIG. 1, the first check valve 21 and/or the second check valve 22 can therefore be arranged in the breathing gas line 4. It can also be possible and reasonable in some embodiments to arrange multiple simple check valves in the breathing gas line 4 (see FIG. 4).

The first check valve 21 can be arranged in the breathing gas line 4 in the flow direction before the fan 3. In the specific exemplary embodiment according to FIG. 1, the first check valve 21 is arranged in the flow direction before the opening of the first fresh gas supply line 7i into the breathing gas line 4. In the specific exemplary embodiment according to FIG. 1, the first check valve 21 is arranged in the flow direction after the opening of the second fresh gas supply line 7ii into the breathing gas line 4.

In the specific exemplary embodiment according to FIG. 1, the first check valve 21 is arranged in the flow direction before the opening of the first O2 flush supply line 11i into the breathing gas line 4. In the specific exemplary embodiment according to FIG. 1, the first check valve 21 is arranged in the flow direction after the opening of the second O2 flush supply line 11ii into the breathing gas line 4.

For example, the first check valve 21 is designed as a simple check valve. It is also conceivable in some embodiments that the first check valve 21 is designed as a spring-loaded check valve (not shown).

The second check valve 22 can be arranged in the expiratory branch 2. The second check valve 22 can be arranged in the breathing gas line 4 in the flow direction after the patient interface.

For example, the second check valve 22 is designed as a simple check valve. It is also conceivable in some embodiments that the second check valve 22 is designed as a spring-loaded check valve (not shown).

The check valves 21, 22 can be configured as simple check valves and can be designed to supervise at least the flow direction of the main flow S. The first check valve 21 can be configured and designed to supervise at least the flow direction of the main flow S and in particular to prevent a backflow of the breathing gas mixture 5 into the reservoir 12. The second check valve 22 can be configured and designed to supervise at least the flow direction of the main flow S and in particular to prevent a backflow of the breathing gas mixture 5 to the patient 90. It is possible to arrange further simple check valves in the breathing gas line 4, as shown in FIG. 4, for example (see below).

The device 100 can additionally comprise the valves 23, 24, which are arranged in or at the breathing gas line 4. The device 100 can, according to the specific exemplary embodiments of FIGS. 1 and 4, comprise at least one first safety valve 23 and/or at least one second safety valve 24.

The first safety valve 23 and/or the second safety valve 24 can be arranged in the breathing gas line 4. The first safety valve 23 and/or the second safety valve 24 can be arranged in the breathing gas line 4 after the fan 3. In the specific exemplary embodiments of FIGS. 1 and 4, the safety valves 23 and 24 are arranged in the breathing gas line 4 directly after the opening of the first O2 flush supply line 11i into the breathing gas line 4 and therefore directly before the patient interface. First safety valve 23 and/or second safety valve 24 are therefore preferably arranged in the inspiratory branch 1.

The safety valves 23, 24 are preferably arranged opposed to one another. The safety valves 23, 24 can be configured and designed to ensure the safety of the device 100 in an emergency situation. An emergency situation can occur, for example, in the event of a power failure, upon low power availability, or in the event of a technical defect of the device 100 or parts of the device 100. The safety valves 23, 24 can moreover ensure the safety of the device 100 if excessively high and/or excessively low pressures prevail in the breathing gas line 4.

It is possible via the safety valves 23, 24 that the patient can independently inhale and/or exhale. It is also possible via the safety valves 23, 24 that the patient can be ventilated manually, for example via manual bag. The independent respiration and/or manual ventilation can then solely take place via the inspiratory branch 1 of the device 100 and the safety valves 23, 24, while bypassing the remaining elements of the device 100.

The first safety valve 23 can be configured as a free breathing valve or as an inspiratory deficiency valve. The first safety valve 23 is preferably designed as a simple check valve. The patient can inhale via the first safety valve 23, for example, if the fan 3 fails and does not supply conveying energy. There is therefore the possibility that the inspiration can take place, for example, via the first safety valve 23. In this case, the inspiration gas can be taken directly from the ambient air.

The second safety valve 24 can be configured as a static overpressure valve. The second safety valve 24 is preferably designed as a spring-loaded check valve. For example, the second safety valve 24 can be an adjustable spring-loaded check valve. The second safety valve preferably additionally has a solenoid, using which the spring can be actuated.

The second safety valve 24 can be subjected to a constant pressure, so that the safety valve 24 remains closed during normal operation of the device 100. The second safety valve 24 can then open if the pressure in the breathing gas line 4 is higher than the operating pressure of the safety valve 24, so that pressure and/or volume can escape via the safety valve 24. The second safety valve 24 can also be opened electronically, for example via energizing the solenoid. The second safety valve 24 can therefore be designed as a mechanical and/or electrical overpressure valve.

If the pressure in the breathing gas line 4 increases, the second safety valve 24 can open passively and/or actively. The pressure in the breathing gas line 4 can be increased, for example, if the expiratory branch is blocked, for example due to a tubing system 92 blocked by soiling or kinking.

There is therefore the possibility that the expiration can take place via the second safety valve 24 in emergency situations. The expiratory breathing gas 5ₑₓₛₚ can in this case be discharged via the inspiratory branch 1 and the second safety valve 24 directly into the ambient air.

The device 100 can comprise at least one further, preferably a plurality of, valves 25, 26, 27, 28, 29. The device 100 can comprise, in the specific exemplary embodiments according to the figures, for example, five further differently arranged valves 25, 26, 27, 28, 29. The valves 25, 26, 27, 28, 29 are designated hereinafter for better clarity as overflow valve 25, discharge valve 26, inlet valve 27, shutoff valve 28, APL valve 29.

The device 100 can comprise at least one overflow valve 25. Alternatively or additionally, the device 100 can comprise at least one discharge valve 26. Alternatively or additionally, the device 100 can comprise at least one inlet valve 27. Alternatively or additionally, the device 100 can comprise at least one shutoff valve 28. Alternatively or additionally, the device 100 can comprise at least one APL valve 29 (airway pressure limiting valve).

Overflow valve 25, discharge valve 26, inlet valve 27, and APL valve 29 are preferably designed as spring-loaded check valves. The shutoff valve 28 is preferably designed as a blockable check valve.

Overflow valve 25, discharge valve 26, inlet valve 27, shutoff valve 28, and APL valve 29 can be set using one or using different adjustable operating pressures. The operating pressures can be set manually and/or automatically. The operating pressures can be preset or adjusted—manually or automatically—in running operation of the device 100. The operating pressures of overflow valve 25, discharge valve 26, inlet valve 27, shutoff valve 28, and APL valve 29 can be predefined using at least one setting.

The inlet valve 27 can be set such that the operating pressure is greater than 0 hPa, for example in a range from 0.1 hPa to 10 hPa. The operating pressure of the inlet valve 27 can preferably be predefined using a setting. In some embodiments, the operating pressure of the inlet valve 27 can also have at least two settings. In the specific exemplary embodiment according to FIG. 1, the inlet valve 27 can be set at a relatively low operating pressure. For example, the operating pressure of the inlet valve 27 can be set to 2 hPa. In such a setting, complete emptying of the reservoir 12 during machine ventilation is avoided since the inlet valve 27 opens and can introduce ambient air into the breathing gas line 4 and/or into the reservoir line 13.

Shutoff valve 28 and/or discharge valve 26 and/or overflow valve 25 can likewise be predefined using a setting. In preferred embodiments, the operating pressures of shutoff valve 28 and/or discharge valve 26 and/or overflow valve 25 can preferably be operated using at least two different operating pressures and accordingly in at least two settings. Operating pressures of shutoff valve 28 and/or discharge valve 26 and/or overflow valve 25 can be set such that the operating pressure is in each case in a range from 0 hPa to 200 hPa. For example, the operating pressures of shutoff valve 28 and/or discharge valve 26 and/or overflow valve 25 can be operated either using at least one relatively high operating pressure or using at least one relatively low operating pressure.

Shutoff valve 28 and/or discharge valve 26 and/or overflow valve 25 can be configured as adjustable between the at least two settings. The operating pressure can preferably be changed manually and/or automatically at least between a first setting and a second setting. The operating pressure can preferably be changed between a rather low operating pressure and a rather high operating pressure. A low operating pressure exists by definition at operating pressures less than 50 hPa. A higher operating pressure exists at operating pressures greater than 50 hPa.

For example, the operating pressure of the shutoff valve 28 and/or the discharge valve 26 and/or the overflow valve 25 can be set in a first setting to 10 hPa or lower, preferably to 5 hPa or lower, for example 2 hPa or lower.

For example, the operating pressure of the shutoff valve 28 and/or the discharge valve 26 and/or the overflow valve 25 can be set in a second setting to 50 hPa or higher, preferably to 100 hPa or higher.

A setting to a rather low operating pressure of, for example, 2 hPa or lower has the effect in the device that the corresponding valve stays in a quasi-open position and therefore enables a gas passage.

A setting to a rather high operating pressure of, for example, 100 hPa or higher has the effect in the device that the corresponding valve stays in a quasi-closed position and therefore does not permit gas passage.

The operating pressure of the APL valve 29 can permit at least one, preferably multiple, particularly preferably a large number of settings. For this purpose, the APL valve 29 can be equipped with a stepping motor, which permits a preferably continuous setting of the spring tension.

Overflow valve 25 and/or discharge valve 26 and/or APL valve 29 can be arranged in or at the conduction system 14. Therefore, in general only expiratory breathing gases 5ₑₓₛₚ are conducted through the overflow valve 25, the discharge valve 26, and the APL valve 29.

In the specific exemplary embodiment according to FIG. 1, the arrangement of the valves is as follows:

The overflow valve 25 can be arranged, for example, in the first line 14i of the conduction system 14. The overflow valve 25 can be arranged between the reservoir 12 and the outlet 14-A. The overflow valve 25 can be configured and designed to supervise the pressure of the reservoir 12. The overflow valve 25 is preferably designed as a spring-loaded check valve. The overflow valve 25 can be configured and designed to discharge pressure from the reservoir 12. The APL valve 29 can be arranged, for example, in the second line 14ii of the conduction system 14. The discharge valve 26 can be arranged, for example, in the third line 14iii of the conduction system 14. Discharge valve 26 and APL valve 29 can be arranged, for example, in lines 14ii, 14iii of the conduction system 14 arranged in parallel. The discharge valve 26 can be configured to conduct gases while bypassing the APL valve.

The inlet valve 27 can be arranged in or at the breathing gas line 4. Ambient air can be fed into the breathing gas line 4 via the inlet valve 27. The inlet valve 27 can be arranged, for example, downstream after the separating means 40 on the breathing gas line 4. The inlet valve 27 can be arranged, for example, downstream before the moisture control module 41 on the breathing gas line 4. The inlet valve 27 can preferably branch off between the chemical separating means 40 and the moisture control module 41. The inlet valve 27 can, for example, optionally feed ambient air into the breathing gas line 4 between the chemical separating means 40 and the moisture control module 41. The feed of the ambient air via the inlet valve 27 can take place at a feed point for ambient air 227 into the breathing gas line 4.

The shutoff valve 28 can be arranged in the breathing gas line 4. The shutoff valve 28 can be arranged, for example, downstream after the separating means 40. The shutoff valve 28 can be arranged, for example, downstream before the moisture control module 41. The shutoff valve 28 can preferably be arranged between the chemical separating means 40 and the moisture control module 41.

The valves 25, 26, 27, 28, 29 are in particular configured and designed to influence the function of the device 100. Via different functionalities and/or settings of the valves 25, 26, 27, 28, 29, for example, the flow path of the breathing gas mixture 5 can be influenced. Via different functionalities and/or settings of the valves 25, 26, 27, 28, 29, for example, it can be controlled whether the breathing gas mixture 5 remains in the circuit and/or is conducted via the conduction system 14 and/or whether ambient air is introduced into the breathing gas mixture 5.

In particular the shutoff valve 28 and/or the APL valve 29 and/or the discharge valve 26 can be configured and designed to supervise the path of the breathing gas mixture 5. Depending on the setting of the operating pressure of the shutoff valve 28 and/or the APL valve 29 and/or the discharge valve 26, the breathing gas mixture 5 can be guided in a closed circuit or in a semi-open circuit.

A closed circuit can be reasonable, for example, if the device 100 is operated in an anesthesia mode using volatile anesthetics VA. A semi-open circuit can be reasonable, for example, if the device 100 is operated in a TIVA mode. A semi-open circuit can also be reasonable if the device 100 is operated in a service mode, for example in a drying mode.

The device 100 can therefore be operated in different operating modes via the settings of the valves 25, 26, 27, 28, 29 and optionally also via the settings of the fan 3 and/or the pressure relief valve 30 and via the operation of the reservoir 12, as described hereinafter. The operating mode of the device 100 can be set manually and/or automatically specified by the control unit 101.

Using the device 100, volatile anesthetics VA can be applied, conducted, and in particular also discharged deliberately for disposal or for reuse. However, ventilation without volatile anesthetics can also be achieved using the device 100.

The device 100 can be operated, for example, in an operating mode which is selected from the following group: anesthesia mode with volatile anesthetics VA; anesthesia mode with intravenously administered anesthetics (TIVA mode); ventilation mode with anesthetics; ventilation mode without anesthetics such as O2 therapy, high flow O2 therapy (HFOT), CPAP, BiLevel, SIMV (synchronized intermittent mandatory ventilation). In principle, all ventilation modes which are typically used in intensive medicine can be implemented using the device 100.

The device 100 offers the advantage that lung-protective ventilation can be implemented even under anesthesia. For example, pressure-controlled ventilation can be carried out, which means a relatively low stress for the lungs. Pressure-controlled ventilation is also advantageous in particular for lung-damaged patients.

The control unit 101 can regulate the device 100 such that the device 100 can be operated in different operating modes. The regulation can take place, for example, via the different setting capability of the components fan 3 and/or valves 25, 26, 27, 28, 29 and/or pressure relief valve 30. In particular, the flow path of the breathing gas mixture 5 and therefore the operating mode of the device 100 as a whole can be influenced via the settings of the operating pressure level of the spring-loaded check valves 25, 26, 27, 28, 29.

The operating mode of the device 100 can be set before start-up or changed in running operation. The change from one to another operating mode can be performed manually by a user, for example by trained medical personnel. The change from one to another operating mode can also be carried out automatically by the control unit 101 if the conditions under the ventilation and/or the anesthesia change.

For example, the device 100 can be operated in an anesthesia mode or in a ventilation mode or a combined anesthesia and ventilation mode. The operating modes can each be operated in different ways, for example as automatic ventilation (machine ventilation), as manual ventilation (hand ventilation), or in the error mode.

Manual ventilation can be or become necessary, inter alia, upon initiation of and/or recovery from a narcosis, during operation in the neck or throat area, and/or in emergency situations.

Machine ventilation can be applied, for example, during an operation using volatile anesthetics and/or intravenously administered anesthetics. Machine ventilation can also be used for ventilation and/or respiratory support of patients who cannot breathe independently or require respiratory support.

Operation in the error mode can be used for current reduction and/or can be necessary in the event of power failure, software crash, or another technical fault. Operation in the error mode offers a safe state in which manual ventilation using at least one fresh gas flow is enabled.

FIGS. 1A to 1G show the device 100 of the first exemplary embodiment in various switches and settings to implement various operating modes. Inactive and/or blocked elements and connections are shown by dashed lines, active and/or free elements and connections are identified by solid lines.

With respect to elements, active means herein that the elements can act as described and/or gases flow through them. With respect to elements, inactive means herein that the elements cannot exert their described function and/or gases cannot flow or can only flow passively through them. With respect to lines, active means herein that gases can be conducted through this line and that the path is not blocked, or at least is not blocked completely. With respect to lines, blocked means herein that gases cannot be conducted through this line and the line is preferably hermetically sealed.

The different functional valves, in particular the overflow valve 25, the discharge valve 26, the inlet valve 27, the shutoff valve 28 can each be set at relatively high operating pressures or at relatively low operating pressures to implement the different operating modes.

A high operating pressure in the sense of the invention means that the respective valve is closed and the gas flow is blocked in the line in which it is located. A high operating pressure can exist, for example, if the corresponding valve is subjected to an operating pressure of greater than 50 hPa, for example 100 hPa.

A low operating pressure in the sense of the invention means that the respective valve is open and can permit the gas flow in the line in which it is located. A low operating pressure can exist, for example, if the corresponding valve is subjected to an operating pressure of less than 10 hPa, for example 2 hPa, or less than 0.5 hPa.

The operating pressure of the APL valve 29 can preferably be set continuously and can permit a large number of settings. The APL valve 29 can therefore restrict and/or prevent the gas flow in the corresponding line.

The APL valve 29 is preferably designed as an electrically settable valve. The APL valve 29 is configured to regulate an inspiration pressure Pinsp. The APL valve 29 can cause breathing gas mixture 5 to be able to be discharged via the outlet 14-A if the pressure in the breathing gas line 4 exceeds the expiratory pressure Pinsp. In preferred embodiments, the APL valve 29 is configured as a controllable loaded check valve having a stepping motor. The APL valve 29 is preferably configured to remain in its last set position without energizing. The APL valve 29 can therefore regulate to the last set inspiration pressure Pinsp without energizing. This is advantageous in particular in the event of a power failure, since the APL valve 29 can thus continue to exert its function.

In preferred exemplary embodiments, the APL valve 29 can be designed as a digital APL valve. The APL valve 29 can preferably be regulated automatically by the software or by the control unit 101. The APL valve can alternatively or additionally also be manually operable. The medical personnel thus have the possibility of operating the APL valve if needed by hand, for example: on the basis of a haptic encoder (rotating handle) and of loading or relieving the APL.

FIG. 1A shows the device 100 in a first operating mode M1 for manual ventilation with application of volatile anesthetics. It is apparent from FIG. 1A that the fan 3 is inactive in the first operating mode M1. An inactive fan 3 means that the fan 3 does not supply conveying energy. The fan wheel of the fan 3 is not driven in the inactive fan. The breathing gas mixture 5 can flow through the fan 3 in an inactive state. In the first operating mode M1, the reservoir 12 supplies the conveying energy for the breathing gas mixture 5. The reservoir 12, which can be designed, for example, in the form of a manual bag, is operated manually or automatically in the first operating mode M1. The reservoir 12 can therefore supply the breathing energy in the first operating mode M1.

Upon operation of the reservoir 12, the breathing gas mixture 5 is conveyed into the reservoir line 13. The overflow valve 25 is subjected to a high operating pressure and blocks the first line 14i. The breathing gas mixture 5 can therefore be introduced from the reservoir line 13 into the inspiratory branch 1 of the breathing gas line 4. The check valve 21 prevents a backflow of the breathing gas mixture 5 into the reservoir line 13.

In the first operating mode M1, the fresh gas module 6 is active. In the first operating mode M1, the fresh gas switching valve 32 is in its base position, so that fresh gas can be introduced via the first fresh gas supply line 7i into the breathing gas line 4. In the first operating mode M1, the first fresh gas supply line 7i is active. In the first operating mode M1, the second fresh gas supply line 7ii is inactive. The fresh gas introduction can then take place in the flow direction after the check valve 21. The fresh gas feed point 207 can then be arranged between the first check valve 21 and the vaporization element 8a (and therefore the anesthetic feed 209). The fresh gas introduction can preferably take place using a constant flow. The fresh gas can then form at least one byflow.

In the first operating mode M1, the anesthetic module 8 is active. In the first operating mode M1, volatile anesthetics VA can be introduced via the anesthetic module 8 and the anesthetic supply line 9 into the vaporization element 8a and thus added to the breathing gas mixture 5 as needed. The breathing gas mixture 5 can be enriched optimally with volatile anesthetics VA via the fresh gas byflow.

In the first operating mode M1, the oxygen module 10 is active. An active oxygen module 10 means that oxygen introduction can take place optionally via the O2 flush 10. In the first operating mode M1, the O2 flush switching valve 31 is in its base position, so that oxygen can optionally be introduced via the O2 flush supply line 11i into the breathing gas line 4. In the first operating mode M1, the first O2 flush supply line 11i is active. In the first operating mode M1, the second O2 flush supply line 11ii is inactive. The oxygen introduction via the O2 flush 10 can optionally take place after (manual or automatic) operation of the O2 flush 10. The oxygen introduction then takes place via the O2 flush 10 and the first O2 flush supply line 11i in the flow direction after the fan 3 and before the safety valves 23, 24. The O2 flush feed point 211 can then be arranged between the fan 3 and the safety valves 23, 24. This offers the advantage that the volatile anesthetics can be washed out rapidly as needed via oxygen flooding of the lungs. The O2 flush 10 can also be used to fill the reservoir 12, for example, after a leakage. The oxygen then flows from the O2 flush 10 via the feed point 211 into the breathing gas line 4 and from there in the flow direction S through the inspiratory branch 1 and the expiratory branch 2 to the reservoir 12.

In the first operating mode M1, the patient can be manually supplied via the inspiratory branch 1 with inspiratory breathing gas 5ᵢₙₛₚ and optionally with volatile anesthetics VA. In the first operating mode M1, moreover the expiratory branch 2 can be active with its elements and can discharge expiratory breathing gas 5ₑₓₛₚ. In the first operating mode M1, the expiratory breathing gas 5ₑₓₛₚ can be conducted away from the patient via the expiratory branch 2. The check valve 22 prevents a backflow of the expiratory breathing gas 5ₑₓₛₚ to the patient. The PEEP valve 30 is active and is set at an individual PEEP.

The main flow direction S of the breathing gas mixture 5 runs via the inspiratory branch 1 in the direction toward the patient interface and from the patient interface into the expiratory branch 2. The sensors 16,17,18,19,20, 39 are active in the first operating mode M1.

The valves 26,28,29 are switched in the operating mode M1 such that the breathing gas mixture 5 can take two different paths in the expiratory branch 2: The breathing gas mixture 54 or components of the breathing gas mixture 5 can be conducted in the direction of the main flow S in the circuit and/or can be discharged from the circuit through the conduction system 14.

The shutoff valve 28 is subjected to a low operating pressure in the first operating mode M1 and releases the breathing gas line 4. The shutoff valve 28 can act as a simple check valve in the first operating mode without action of the solenoid. The breathing gas mixture 5 can thus be conducted at least partially in the direction of the main flow S in the circuit in the operating mode M1. The breathing gas mixture 5 is then introduced from the expiratory branch 2 back into the inspiratory branch 1. The breathing gas mixture 5 passes the separating means 40 and the moisture control module 41 in this case, which are active in the first operating mode M1. CO2 is separated in the separating means 40 and moisture can be withdrawn from the breathing gas mixture 5 in the moisture module 41. Fresh gas and/or oxygen and/or anesthetics can be supplied to the breathing gas mixture 5 again in the inspiratory branch 1.

It is possible to influence whether the second line 14ii and the third line 14iii are active and are therefore gas-carrying by way of the settings of the discharge valve 26 and the APL valve 29. It is apparent from FIG. 1A that in the first operating mode M1, the second line 14ii is active and the third line 14iii is blocked.

The discharge valve 26 is subjected to a high operating pressure in the first operating mode M1 and blocks the third line 14iii. the APL valve 29 is subjected to an operating pressure in the first operating mode M1 which releases the gas flow through the second line 14ii to a restricted extent. The breathing gas mixture 5 can thus be discharged from the circuit at least partially through the second line 14ii of the conduction system 14.

The APL valve 29 can be set to a patient-specific inspiration pressure Pinsp. The patient-specific inspiration pressure Pinsp can be determined and set beforehand. In healthy adults, the inspiration pressure Pinsp is generally between 20 and 25 hPa. With previously damaged lungs and/or during the operation, a higher inspiration pressure Pinsp can be or become necessary. The inspiration pressure Pinsp is pre-settable and also settable during use. The setting of the APL valve 29 can be controlled manually and/or by the control unit 101.

The APL valve 29 can limit the maximum pressure during the inspiration by discharging a partial gas from the circuit. Moreover, it can be ensured via the discharge of a partial gas that a gas exchange takes place. Metabolites of the lungs (such as methane or ammonia) can be removed from the circuit via the gas exchange. Fresh gas and/or oxygen and/or anesthetics can be supplied to the circuit depending on need.

The reservoir 12 is actively operated during the inspiration. The breathing gas mixture 5 reaches the patient via the Y-piece 93 until the inspiration pressure Pinsp set at the APL valve 29 is reached in the patient. An inspiratory plateau can then be maintained in which no flow flows from or to the patient.

During the expiration, the operation of the reservoir 12 is ended. The breathing gas mixture 5 reaches the expiratory branch 2 from the patient via the Y-piece 93, the pressure drops to the PEEP set at the pressure control valve 30. There is then an expiratory plateau, in which no flow flows from or to the patient.

An excess fresh gas flow is always used during a manual ventilation. The excess volume can now be released in the inspiration into the conduction system 14. The reservoir 12 then always has to be used more than only for the patient volume.

FIG. 1B shows the device 100 in a second operating mode M2 for automatic ventilation with application of volatile anesthetics. It is apparent from FIG. 1B that the fan 3 is active in the second operating mode M2. The active fan 3 supplies the conveying energy for the breathing gas mixture 5. The fan wheel of the fan 3 is driven and conveys the breathing gas mixture 5. The fan 3 can generate the required patient flow and a constant byflow in the second operating mode M2. The fan 3 can generate a set flow pattern in the second operating mode M2. In the second operating mode M2, the reservoir 12 is used as a store for a part of the breathing gas mixture 5. In the second operating mode M2, the reservoir 12 does not supply conveying energy for the breathing gas mixture 5. In the second operating mode M2, the reservoir 12 can at least partially supply the volume for the fan 3.

In the second operating mode M2, the fresh gas switching valve 32 and the O2 flush switching valve 31 are in the base position, so that fresh gas can be introduced via the first fresh gas supply line 7i and optionally oxygen can be introduced via the first O2 flush supply line 11i into the breathing gas line 4. The fresh gas introduction can take place in the second operating mode M2 in the flow direction after the check valve 21. The fresh gas introduction can preferably take place with a constant flow. The fresh gas forms a byflow together with the fan flow.

The anesthetic module 8 is active in the second operating mode M2. In the second operating mode M2, volatile anesthetics VA can be introduced via the anesthetic module 8 and the anesthetic supply line 9 into the vaporization element 8a and thus added to the breathing gas mixture 5 as needed. The breathing gas mixture 5 can be enriched optimally with volatile anesthetics VA via the byflow.

In the second operating mode M2, the patient can be supplied via the inspiratory branch 1 with inspiratory breathing gas 5ᵢₙₛₚ and optionally with volatile anesthetics. In the second operating mode M2, the expiratory branch 2 can moreover be active, so that the expiratory breathing gas 5ₑₓₛₚ is conducted away from the patient via the expiratory branch 2. The check valves 21, 22 prevent a backflow of the breathing gas mixture 5 against the main flow direction S. The PEEP valve 30 is active and is set at an individual PEEP.

The main flow direction S of the breathing gas mixture 5 runs via the inspiratory branch 1 in the direction toward the patient interface and from the patient interface via the expiratory branch 2. The breathing gas mixture 5 is then introduced from the expiratory branch 2 back into the inspiratory branch 1 and/or the reservoir 12.

The breathing gas mixture 5 largely flows into the patient in the inspiration, until an inspiratory plateau is reached. The byflow flows through the expiratory branch 2. Upon reaching the inspiratory plateau, the fan 3 predominately conveys the byflow which flushes the circuit K1. The byflow refills the reservoir 12 in this phase.

The valves 26, 28, 29 are switched in the second operating M2 such that the breathing gas mixture 5 essentially runs in the circuit along the main flow direction S: The shutoff valve 28 is subjected to a low operating pressure in the second operating mode M2 and releases the breathing gas line 4. The APL valve 29 is subjected to a higher operating pressure than the shutoff valve 28 and thus blocks the second line 14ii. the discharge valve 26 is subjected to a high operating pressure and blocks the third line 14iii.

The breathing gas mixture 5 can thus be conducted in the circuit in the direction of the main flow S in the second operating mode M2. The breathing gas mixture 5 is then introduced from the expiratory branch 2 back into the inspiratory branch 1. The breathing gas mixture 5 passes the separating means 40 and the moisture control module 41 in this case. CO2 is separated in the separating means 40 and moisture can be withdrawn from the breathing gas mixture 5 in the moisture module 41. Fresh gas and/or oxygen and/or anesthetics can be supplied to the breathing gas mixture 5 again in the inspiratory branch 1.

In the second operating mode M2, a low operating pressure is applied to the overflow valve 25 and it can release the first line 14i. The breathing gas mixture 5 or components of the breathing gas mixture 5 can thus be conducted through the first line 14i of the conduction system 14 to the outlet 14-A and emitted to the surroundings. When the reservoir 12 is full, thus when the pressure of the reservoir 12 exceeds the set pressure of the overflow valve 25, breathing gas can be discharged from the reservoir 12 to the outlet 14-A via the first line 14i and through the overflow valve 25. Overfilling 12 of the reservoir 12 can thus effectively be prevented. Overfilling of the reservoir 12 could damage it and/or cause it to burst.

The inlet valve 27 can be designed as a volume and/or flow deficiency valve. The inlet valve 27 can be set to a low operating pressure. The inlet valve 27 is preferably set such that outside air can be supplied to the circuit in case of a negative pressure in the breathing gas line 4. In the second operating mode M2, a negative pressure can arise in the breathing gas line 4, for example in the event of an (undesired or desired) leakage. Operating the fan 3 when a leakage is present in the system can cause the reservoir 12 to run empty and a negative pressure to arise in the breathing gas line 4. A negative pressure in the breathing gas line 4 can open the inlet valve 27, so that ambient air can be supplied to the breathing gas line 4 via the inlet valve 27. For example, the inlet valve 27 is set at an operating pressure of 2 hPa. This corresponds to the pressure of the filled reservoir 12.

If one considers FIGS. 1A and 1B, for example, it is shown that the breathing gas mixture 5 can be conducted in an essentially closed circuit and/or in an essentially semi-open circuit. The cooperation of the shutoff valve 28, the discharge valve 26, and the APL valve 29 is essential for this purpose. The cooperation of the valves 26, 28, 29 can take place as follows:

With a low set operating pressure of the shutoff valve 28 and a high set operating pressure of the discharge valve 26 and the APL valve 29, the breathing gas mixture 5 can be guided in an essentially closed circuit. The closed circuit exists when the operating pressure of the shutoff valve 28 is less than the operating pressure of the discharge valve 26 and the APL valve 29. Because the respective operating pressure of discharge valve 26 and APL valve 29 is higher than the operating pressure of the shutoff valve 28, the path to the outlet 14-A is blocked. Because the respective operating pressure of discharge valve 26 and APL valve 29 is higher than the operating pressure of the shutoff valve 28, the path of the breathing gas line 4 to the separating means 40 and/or to the moisture control module 41 is open.

With a high operating pressure of the shutoff valve 28 and low operating pressures in each case of discharge valve 26 and/or APL valve 29, the breathing gas mixture 5 can be guided in a semi-open circuit. The semi-open circuit exists when the operating pressure of the shutoff valve 28 is greater than the respective operating pressure of discharge valve 26 and APL valve 29. In this case, the shutoff valve 29 blocks the path to the fan 3. The flow direction of the breathing gas mixture 4 then runs—roughly outlined—from the fan 3 via the inspiratory branch 1 to the patient interface and to the patient 90 and from the patient 90 via the expiratory branch 2 to the outlet 14-A. Because the operating pressure of the shutoff valve 28 is higher than the respective operating pressures of discharge valve 26 and APL valve 29, the path to the chemical separating means 40 or to the fan 3 is blocked. Because the operating pressure of the shutoff valve 28 is higher than the respective operating pressures of discharge valve 26 and APL valve 29, the path to the outlet 14-A is open.

FIG. 1C shows the device 100 in a third operating mode M3 for manual ventilation without application of volatile anesthetics. FIG. 1D shows the device 100 in a fourth operating mode M4 for automatic ventilation without application of volatile anesthetics.

Alternatively or additionally to the use with volatile anesthetics, the device 100 can be operated in operating modes without an application of volatile anesthetics. The device 100 is therefore also designed and configured to ventilate the patient without volatile anesthetics. The device 100 can therefore also be used with application of total intravenous anesthesia (TIVA) and/or with application of local anesthesia such as a spinal anesthesia. In a TIVA, anesthetics, such as propofol, are injected into the blood circulation. Additional anesthesia using inhalation narcotics is possible. The device 100 offers the advantage that it permits ventilation in combination with total intravenous anesthesia, wherein alternatively or additionally volatile anesthetics VA can also be supplied via the airways. In operating modes without the addition of inhalation anesthetics VA, the device 100 can be operated, for example, in an open and/or semi-open circuit. In operation without inhalation anesthetics VA, the breathing gas can be discharged into the surroundings. In operating modes without the addition of inhalation anesthetics VA, the breathing gas mixture can be completely exchanged in each breathing phase (inspiration and expiration). The minute volume is thus always newly provided. For the treatment of an adult, this means that on average approximately 6 L/min of breathing gas mixture are provided and applied.

FIG. 1C shows that the fan 3 is inactive in the third operating mode M3, so that only the breathing gas mixture 5 flows through the fan 3. In the third operating mode M3, the reservoir 12 supplies the conveying energy for the breathing gas mixture 5 and therefore the breathing energy.

In the third operating mode M3, the anesthetic module 8 is generally inactive. In the third operating mode M3, in general no volatile anesthetics VA are added to the breathing gas mixture 5.

During operation of the reservoir 12, the breathing gas mixture 5 is conveyed in the reservoir line 13. From the reservoir line 13, the breathing gas mixture 5 is introduced into the inspiratory branch 1 of the breathing gas line 4. The check valve 21 prevents a backflow of the breathing gas mixture 5 into the reservoir line 5.

in the third operating mode M3, the fresh gas switching valve 32 can preferably be switched such that fresh gas can be introduced via the second fresh gas supply line 7ii into the breathing gas line 4. The fresh gas introduction can then take place in the flow direction before the first check valve 21.

In the third operating mode M3, the O2 flush switching valve 31 can preferably be switched such that oxygen can optionally be introduced via the second O2 flush supply line 11ii into the breathing gas line 4. The oxygen introduction via the O2 flush 10 can optionally take place after (manual or automatic) operation of the O2 flush 10. The oxygen introduction via the O2 flush 10 and the second O2 flush supply line 11ii then takes place in the flow direction before the fan 3. The oxygen introduction via the O2 flush 10 preferably takes place in the third operating mode M3 in the flow direction before the first check valve 21.

In the third operating mode M3, fresh gas and/or oxygen can therefore be fed before the first check valve 21 into the breathing gas line 4 and therefore fill the reservoir 12 if needed.

In the third operating mode M3, the patient can be manually supplied with inspiratory breathing gas 5ᵢₙₛₚ at least via the inspiratory branch 1. In the third operating mode M3, the expiratory branch 2 with its elements can moreover be active. In the third operating mode M3, the expiratory breathing gas 5ₑₓₛₚ can be conducted away from the patient via the expiratory branch 2. The PEEP valve 30 is active in this case and is set at an individual PEEP. The PEEP valve 30 can regulate to the set PEEP pressure.

The main flow direction S of the breathing gas mixture 5 runs via the inspiratory branch 1 in the direction toward the patient interface and from the patient interface via the expiratory branch 2. In the operating mode M3, in general volatile anesthetics VA are not applied, so that the breathing gases do not have to be guided in a closed circuit. In the operating mode M3, the expiratory breathing gas 5ₑₓₛₚ can be discharged via the expiratory branch 2 and the conduction system 14 into the surroundings. The breathing gas mixture 5 is guided in the third operating mode M3 in a semi-open circuit, i.e. the inspiration runs via the inspiratory branch 1 and the expiration runs via the expiratory branch 2, wherein the expiratory breathing gas 5ₑₓₛₚ is discharged via the conduction system 14 and is not supplied to the inspiratory branch 1 again.

For this purpose, the shutoff valve 28 can be subjected to a high operating pressure in the third operating mode M3 and block the connection between inspiratory branch 1 and expiratory branch 2 such that the expiratory breathing gas 5ₑₓₛₚ cannot be introduced back into the inspiratory branch. Breathing gases do not flow through the separating means 40 and the moisture control module 41 in the third operating mode M3 and they are inactive.

In the third operating mode M3, the discharge valve 26 is subjected to a low operating pressure and releases the third line 14iii. The APL valve 29 is subjected to a higher operating pressure than the discharge valve 26 and therefore blocks the second line 14ii.

Due to the low operating pressure of the discharge valve 26, the expiratory breathing gas 5ₑₓₛₚ is discharged via the third line to the outlet 14-A and into the surroundings while bypassing the APL valve 29. In the third operating mode M3, the APL valve 29 is inactive, so that the PEEP valve 30 also specifies the inspiration pressure Pinsp. The PEEP corresponds in the third operating mode M3 to the inspiration pressure Pinsp. During the expiration, the PEEP valve 30 regulates to the PEEP pressure. During the inspiration, the PEEP valve 30 regulates the inspiratory patient pressure.

In the third operating mode M3, the breathing gas mixture 5 is not processed, but rather all of the gas used is released to the surroundings via the conduction system 14.

FIG. 1D shows that the breathing gas mixture 5 is guided in a semi-open circuit in the fourth operating mode M4 similarly to the third operating mode M3. The flow path of the fourth operating mode M4 essentially corresponds to that of the third operating mode M3.

In contrast to the third operating mode, the fan 3 is active in the fourth operating mode M4 and supplies the conveying energy for the breathing gas mixture 5. In the fourth operating mode M4, the fan 3 can generate the required patient flow and a constant byflow. The fan 3 can dynamically generate a set patient flow pattern in the fourth operating mode M4. In the fourth operating mode M4, the reservoir 12 can at least partially supply the volume for the fan 3. The first gas introduction can preferably take place using a constant flow. The fresh gas forms a byflow together with the fan flow.

A negative pressure is generated before the fan 3 in the inspiratory branch 1 by the active fan 3, so that in the fourth operating mode M4, the inlet valve 27 can be activated. If the fresh gas and/or oxygen feed is too low, ambient air can therefore be conveyed in the breathing gas line 4 via the inlet valve 27.

In the fourth operating mode M4, the overflow valve 25 is subjected to a low operating pressure and can release the first line 14i. The breathing gas mixture 5 can therefore be conducted at least partially through the first line 14i to the outlet 14-A and discharged to the surroundings. The overflow valve 25 can be configured and designed to protect the reservoir 12 from distention and/or destruction.

In the fourth operating mode M4, fresh gas can be fed via the fresh gas module 6 and/or oxygen can be fed via the O2 flush 10 before the first check valve 21 into the breathing gas line 4 and rapidly fill the reservoir 12 if needed. Overfilling of the reservoir 12 can be effectively prevented by the overflow valve 25.

FIG. 1E shows the device 100 in a fifth operating mode M5 for manual ventilation in an emergency mode.

The device 100 is configured and designed to also be able to be safely operated in emergency situations using the fifth operating mode M5. An emergency situation can occur, for example, if the primary power source 103 and/or the secondary power sources 104 fail. An emergency situation can also occur, for example, if the primary power source 103 and/or the secondary power sources 104 can only provide energy to a restricted extent. In an emergency situation, the power supply can be available to a reduced extent or can fail entirely. In the fifth operating mode M5, the device 100 can be operated without power and/or in a power-saving manner. In the fifth operating mode M5, the device 100 can be operated without power source and/or only using the secondary power sources 104.

An emergency situation can alternatively or additionally also occur if critical components of the device 100, such as the control unit 101, the software, hardware, or the fan 3, fail in parts or as a whole.

In the fifth operating mode M5, the device 100 can preferably maintain the last selected functions and settings and continue to enable ventilation. The medical personnel remain capable of action even in emergency situations via the fifth operating mode M5. The device 100 is configured and designed to switch automatically to the fifth operating mode M5 upon occurrence of an emergency situation in order to secure an emergency supply. It is also possible to manually set the fifth operating mode M5. This can be advantageous to establish power-saving operation.

In the fifth operating mode M5, the monitor can be set dark to save power. In the fifth operating mode M5, all actuators can be deenergized. In the fifth operating mode M5, the sensors can be inactive. In the fifth operating mode M5, the fan 3 can also be put into power-saving operation. For example, the fan can be operated less dynamically.

The device 100 can be configured and designed to apply volatile anesthetics VA in the fifth operating mode M5, since the fresh gas flow can remain.

In preferred embodiments, no volatile anesthetics VA are applied in the fifth operating mode M5. The anesthetic module 8 and the vaporization element 8a are inactive, so that the patient can still be ventilated using the device 100, but without application of volatile anesthetics. Anesthesia using intravenous anesthetics is then possible.

It is apparent from FIG. 1E that the fan 3 can be inactive in the fifth operating mode M5. In the fifth operating mode M5, the reservoir 12 can supply the conveying energy for the breathing gas mixture 5. The breathing gas mixture 5 can (passively) flow through the fan 3. Upon operation of the reservoir 12, the breathing gas mixture 5 is conveyed in the reservoir line 13. From the reservoir line 13, the breathing gas mixture 5 is introduced into the inspiratory branch 1 of the breathing gas line 4. The check valve 21 prevents a backflow of the breathing gas mixture 5 into the reservoir line 13.

When deenergized, the fresh gas switching valves 32 are in their base position. Therefore, in the fifth operating mode M5, fresh gas can be fed via the first fresh gas supply line 7i into the breathing gas line 4. The feed of fresh gas can therefore take place at the feed point 207 and in the flow direction after the check valve 21.

The fresh gas can be fed in the operating mode M5 with a constant flow into the breathing gas line, since the switching valve 87 and/or the dosing valve 88 of the fresh gas supply line 7 can remain when deenergized in their last set positions. The dosing of fresh gas can therefore remain at the last set value.

In the fifth operating mode M5, the O2 flush 10 can be manually operated so that oxygen can optionally be fed into the breathing gas line 4. When deenergized, the O2 flush switching valves 31 are in their base position. The feed of oxygen can therefore take place via the first O2 flush supply line 11i. The feed of oxygen can therefore take place at the feed point 211 and in the flow direction before the safety valves 23, 24.

The patient can therefore be supplied with breathing gas and/or fresh gas and/or oxygen in the fifth operating mode M5 via an operation of the reservoir 12 and via the fresh gas module 6 and/or the oxygen module 10.

FIG. 1E shows that the expiratory breathing gas 5ₑₓₛₚ is guided similarly in the fifth operating mode M5 as in the first operating mode M1. The flow path of the fifth operating mode M5 essentially corresponds to that of the first operating mode M1: The valves 26, 28, 29 are switched in the fifth operating mode M5 such that the breathing gas mixture 5 can take two different paths in the expiratory branch 2: The breathing gas mixture 5 or components of the breathing gas mixture 5 can be conducted in the direction of the main flow S in the circuit and/or discharged from the circuit by the conduction system 14. The breathing gas mixture 5 can be able to be conducted at least in the first circuit K1 in the emergency mode M5. The breathing gas mixture 5 can be able to be at least partially discharged via the outlet 14-A in the emergency mode M5.

The shutoff valve 28 is deenergized and therefore inactive in the fifth operating mode M5. Therefore, the shutoff valve 28 acts as a simple check valve and releases the breathing gas line 4 in the direction of the main flow S. The expiratory breathing gas 5ₑₓₛₚ can therefore be conducted at least partially in the circuit in the direction of the main flow S. In this case, the expiratory breathing gas 5ₑₓₛₚ passes the separating means 40, in which CO2 can be chemically separated. The moisture management 41 is inactive when deenergized and only has flow through it. Fresh gas and/or oxygen can be supplied to the breathing gas mixture 5 again in the inspiratory branch 1.

The third line 14iii of the conduction system 14 is blocked by the discharge valve 26 in the fifth operating mode M5. When deenergized, the discharge valve 26 is subjected to a high operating pressure. When deenergized, the discharge valve 26 can have an operating pressure of 100 hPa, for example, due to the spring tension.

The second line 14ii of the conduction system 14 can be active in the fifth operating mode M5, since the APL valve 29 remains at the last set position when deenergized. The APL valve 29 can therefore still regulate to the (last set) inspiration pressure Pinsp in the emergency mode M5.

Since the operating pressure of the APL valve 29 in the fifth operating mode M5 is lower than the operating pressure of the discharge valve 26, the expiratory breathing gas 5ₑₓₛₚ can be discharged at least partially through the second line 14ii and the outlet 14-A.

The gas flow through the second line 14ii can therefore be released to a restricted extent in the fifth operating mode M5 by the APL valve 29. Excess breathing gas mixture 5 which cannot be guided in the circuit of the breathing gas line 4 can be discharged during the inspiration via the second line 14ii.

In the fifth operating mode M5, the pressure control valve 30 is deenergized and therefore no longer adjustable. In the fifth operating mode M5, the pressure control valve 30 can passively regulate the expiration pressure Pexsp. When deenergized, the pressure control valve 30 drops to its passive operating pressure. The pressure control valve 30 can in the fifth operating mode M5 regulate the PEEP passively to 5 hPa, for example.

In contrast to the first operating mode M1, the sensors 15, 16, 17, 18, 19, 20, 39 and the moisture control module 41 are deenergized and therefore inactive in the fifth operating mode M5.

Alternatively or additionally to the above-described operating modes, the device 100 can also be operated in at least one sixth operating mode M6, which can provide a large, essentially constant flow. A constant flow can be used, for example, to carry out a high flow therapy (HFOT). The device 100 can be used to carry out a HFOT. For this purpose, humidifiers and/or heating elements (not shown here) can optionally be connected to the device 100 in order to humidify and/or heat the breathing air.

The sixth operating mode M6 can be used in combination with a spinal anesthesia and/or with other local anesthesia. The sixth operating mode M6 can be operated with conscious patients who are to receive respiratory assistance.

FIG. 1F shows the device 100 in the sixth operating mode M6 for a constant flow (HFOT), wherein optionally volatile anesthetics VA can be applied.

The sixth operating mode M6 can be implemented both without addition of volatile anesthetics (see FIG. 1F) and with an application of volatile anesthetics (not shown). The sixth operating mode M6 can also be implemented in an error mode (not shown).

FIG. 1F shows the device 100 in the sixth operating mode for constant flow and without application of volatile anesthetics. The device 100 is configured and designed to generate a nearly constant flow without generating or specifying a breathing pattern. The flow is applied in the sixth operating mode M6 independently of the breathing phases of the patient.

In the HFOT modes, flows of up to 80 l/min can be achieved. In the HFOT mode, in general constant flows of 60 l/min are generated. The HFOT can take place via an open and/or semi-open circuit.

The HFOT is preferably implemented via an open circuit without expiratory branch 2 (see FIG. 1F). The device 100 can then only have an inspiration tube. In this case, the patient 90 can be supplied with the breathing gas mixture 5 via the inspiration tube and the patient interface 91 and can exhale into the surroundings. The patient interface 91 is preferably a nasal cannula to implement a HFOT.

It is apparent from FIG. 1F that in the sixth operating mode M6 neither is the fan 3 active nor does the reservoir 12 supply the conveying energy. The breathing gas mixture 5 is exclusively driven by the fresh gas module 6.

In the sixth operating mode M6, the fresh gas switching valves 32 are in their base position. Therefore, in the sixth operating mode M6 fresh gas can be fed via the first fresh gas supply line 7i into the breathing gas line 4. The feed of fresh gas can therefore take place at the feed point 207 and in the flow direction after the check valve 21. The feed of fresh gas can preferably supply the flow for the HFOT. The constant fresh gas flow can preferably be conducted without influence directly to the patient interface.

Optionally, in the sixth operating mode M6, the O2 flush 10 can be manually operated and can optionally feed additional oxygen into the breathing gas line 4. In the sixth operating mode M6, the O2 flush switching valves 31 are in their base position. The feed of oxygen can therefore take place via the first O2 flush supply line 11i. The feed of oxygen can therefore take place at the feed point 211 and in the flow direction before the safety valves 23, 24.

The inspiratory pressure sensor 15 and/or the inspiratory flow sensor 17 and/or the oxygen sensor 19 is preferably active in the sixth operating mode M6. The second safety valve 24 is electrically activatable in the sixth operating mode M6 and offers additional protection.

The device 100 is alternatively or additionally configured to provide a constant flow and apply volatile anesthetics VA simultaneously (not shown) in the sixth operating mode M6.

A constant flow is also generated with an application of volatile anesthetics, without generating or specifying a breathing pattern and independently of the breathing phases of the patient. In the sixth operating mode M6, the anesthetic module 8 and/or the vaporization element 8a can optionally be activated. A constant fresh gas flow is fed via the fresh gas module 6 and the first fresh gas supply line 7i, which can be enriched with anesthetics VA after the feed into the vaporization element 8a and can be conducted without further influence directly to the patient interface.

The device 100 can then be configured and designed to be connected to a manual bag 35 designed for this purpose, so that the volatile anesthetics are not emitted into the surroundings, but rather can be reused via the manual bag 35 (not shown).

During an application of volatile anesthetics in the sixth operating mode M6, the multigas sensor 20 can moreover preferably be active. Moreover, for reasons of safety an O2 flush close to the patient is possible.

The device 100 is alternatively or additionally configured to provide a constant flow in an error mode without an application of volatile anesthetics (not shown).

In the error mode of the sixth operating mode M6, thus, for example, in a deenergized state, the fresh gas feed can be continued after the first check valve 21, since the switching valve 87 and/or the dosing valve 88 of the fresh gas supply line 7 can remain when deenergized in their last set positions.

In the error mode of the sixth operating mode M6, the sensors 15, 17, 19 can be inactivated. In the error mode, no measurements of the inspiratory flow sensor 17 and/or the O2 sensor 19 and/or the inspiratory pressure sensor 15 then take place.

In the error mode of the sixth operating mode M6, the anesthetic module 8 can be inactivated manually or automatically so that anesthetics are no longer fed. The O2 flush 10 can still be manually operated in the error mode of the sixth operating mode M6, so that oxygen can optionally be fed close to the patient into the breathing gas line 4.

FIG. 1G shows the device 100 in a seventh operating mode M7, which represents a service mode. In the seventh operating mode M7, the device 100 is generally not connected to a patient 90.

The device 100 can be operated alternatively to the above-described operating modes in the seventh operating mode M7, which permits maintenance and/or service and/or drying. For example, the device 100 can also be operated in a standby mode in this way. The device 100 can advantageously be operated in the seventh operating mode M7 to enable drying of the device 100 or at least parts of the device 100. The moisture condensed due to the use of the chemical separating means 40 can be effectively removed from the breathing gas line 4 in this way.

In the seventh operating mode M7, the fresh gas module 6 is inactive. In the seventh operating mode M7, the fresh gas module 6 can be inactivated by closing the dosing valve 88 and/or the switching valve 87 in the fresh gas supply line 7.

In the seventh operating mode M7, the fan 3 is active. The fan 3 can generate a constant pressure and/or a constant flow and/or a constant volume. The fan 3 can generate a negative pressure in the breathing gas line 4 before the fan 3 in the flow direction. Since the fresh gas module 6 is inactive, it does not supply fresh gas for the fan 3. Therefore, in the seventh operating mode M7, the inlet valve 27 can open. Therefore, in the seventh operating mode M7, air can be aspirated from the surroundings into the device 100 via the inlet valve 27. The feed of the ambient air via the inlet valve 27 can take place at the feed point for ambient air 227 into the breathing gas line 4.

The feed point for ambient air 227 can be located in the flow direction after the separating means 40 in the specific exemplary embodiment according to FIG. 1. In this exemplary embodiment, the feed point for ambient air 227 can preferably be located in the flow direction after the shutoff valve 28. The feed point for ambient air 227 is located, for example, in the flow direction before the moisture control module 41. In the specific exemplary embodiment according to FIG. 1, the feed point for ambient air 227 is located between the shutoff valve 28 and the moisture control module 41.

The aspirated ambient air is therefore conducted through the moisture control module 41. In the seventh operating mode M7, the moisture control module 41 is inactive and the aspirated ambient air can flow passively through it. The ambient air can absorb moisture in the moisture control module 41. The ambient air then flows through the inspiratory branch 1 and expiratory branch 2. The Y-piece 93 is closed on the patient side in the seventh operating mode M7. The ambient air can moreover flow through the first check valve 21, the fan 3, the inspiratory flow sensor 17, the tubing system 92, the second check valve 22, the PEEP valve 30, the expiratory flow sensor 18, and/or the discharge valve 27, wherein the ambient air can absorb moisture and can be discharged via the outlet 14-A.

In the seventh operating mode M7, the discharge valve 26 is subjected to a low operating pressure and releases the third line 14iii. The APL valve 29 is subjected to a higher operating pressure than the discharge valve 26 and therefore blocks the second line 14ii.

Due to the low operating pressure of the discharge valve 26, the moisture-saturated ambient air is discharged via the third line 14iii to the outlet 14-A and into the surroundings while bypassing the APL valve 29.

FIG. 4 shows a schematic structure of the device in a second example embodiment.

The exemplary embodiment of FIG. 4 corresponds in its essential structure to that of the exemplary embodiment of FIG. 1, but can comprise at least the at least one mechanical separating means 60. The separating means 60 can be designed as a mechanical CO2 absorber 60. The CO2 separation can take place mechanically via at least one diffusion filter 61 as described below with respect to FIG. 4. The mechanical separating means 60 can be comprised alternatively or additionally to the chemical separating means 40. However, the device 100 preferably comprises either the chemical separating means 40 (see FIG. 1) or the mechanical separating means 60 (see FIG. 4).

The mechanical separating means 40 can be arranged in or at the breathing gas line 4. The mechanical separating means 40 can be arranged downstream after the patient interface. The mechanical separating means 40 is preferably arranged in the expiratory branch 2 of the breathing gas line 4. The mechanical separating means 40 can be arranged downstream after the multigas sensor 20. The mechanical separating means 40 can be arranged downstream after the expiratory pressure sensor 16.

The mechanical separating means 40 can be arranged upstream before the conduction system 14. The mechanical separating means 40 can be arranged upstream before the PEEP valve 30. The mechanical separating means 40 can be arranged upstream before the opening of the bypass 75 into the expiratory branch 2. The mechanical separating means 40 can be arranged upstream before the second safety valve 22.

In the specific exemplary embodiment according to FIG. 3, the mechanical separating means 40 can be arranged between the opening of the bypass 75 into the expiratory branch 2 and the patient interface.

The device 100 in the exemplary embodiment of FIG. 4 can comprise further additional elements in relation to the exemplary embodiment of FIG. 1, which enable or at least positively influence the functionality of the device 100 with the mechanical separating means 60. The device 100 in the exemplary embodiment of FIG. 4 can also comprise fewer elements in relation to the exemplary embodiment of FIG. 1. For example, the exemplary embodiment according to FIG. 4 can be designed without chemical absorber 40. Therefore, the moisture control module 41 also no longer has to be comprised. In alternative embodiments, the device 100 can also be equipped with a mechanical separating means 60 and with a moisture control module 41 (not shown).

The device 100 can comprise at least one sweep gas module 70 and at least one sweep gas supply line 71 for providing, introducing, discharging, and supervising sweep gas 64 for the mechanical separating means 60 (see also below herein, FIG. 5). The sweep gas module 70 is configured and designed to provide sweep gas 64 for the separating means 60. Sweep gas 64 can be introduced into and/or discharged from the mechanical separating means 60 in a supervised manner via the sweep gas supply line 71. The sweep gas supply line 71 is pneumatically connected to the separating means 60 for this purpose.

The device 100 can moreover comprise at least one bypass 75. The bypass 75 can be designed as a gas-carrying line and can be pneumatically connected to the breathing gas line 4. At least a part of the breathing gas mixture 5 can be conducted via the bypass 75. The bypass 75 can permit or enable a bypass flow S1, the direction of which is shown indicated by a dashed line in FIG. 4. In the exemplary embodiment of FIG. 4, the bypass flow S1 can exist in addition to the main flow S of the breathing gas mixture 5.

The bypass 75 is configured to at least temporarily establish a connection conducting breathing gas from the inspiratory branch 1 to the expiratory branch 2 such that a second circuit K2 is formed, in which the breathing gas mixture 5 can be conducted. The second circuit K2 can at least partially correspond to the first circuit K1. In this case, the bypass 75 is arranged such that the fitting 93 for a patient interface is not arranged in the second circuit K2. The fitting 93 for a patient interface is only arranged in the first circuit K1. Moreover, the bypass 75 is arranged such that the mechanical separating means 60 is not arranged in the second circuit K2. The mechanical separating means 60 is only arranged in the first circuit K1.

The breathing gas mixture 5 can be conducted in the first circuit K1 with the main flow S and additionally can be conducted in the second circuit K2 with the bypass flow S1. Bypass flow S1 and main flow S of the breathing gas mixture 5 can run in parallel at least in some areas.

At least one valve 76 can be arranged in the bypass 75. The valve 76 can be designed as a check valve. The valve 76 can then at least specify the direction of the bypass flow S1. The valve 76 can in particular prevent a backflow of the bypass flow S1 into the inspiratory branch 1 of the breathing gas line 4. In some embodiments, the valve 76 can be designed as a simple check valve or as a loaded check valve (not shown).

In preferred embodiments, the valve 76 can be designed as a blockable check valve and therefore as a bypass shutoff valve 76. In the specific exemplary embodiment of FIG. 4, for example, the bypass shutoff valve 76 can be designed as a blockable check valve having a solenoid. The bypass shutoff valve 76 can therefore preferably be operated in at least two settings: In a first setting, for example in an idle setting, the bypass shutoff valve 76 can release the flow in one direction and block it in one direction. In this first setting, the bypass shutoff valve 76 can act as a simple check valve. In a second setting, for example in an active position, the bypass shutoff valve 76 can block the flow in both directions. The active position of the valve 76 can be established, for example, by energizing the solenoid.

The bypass 75 can branch off in the inspiratory branch 1. The bypass 75 can open into the expiratory branch 2. The bypass 75 can branch off between the fan 3 and the patient interface 91. The bypass 75 can preferably branch off between the fan 3 and the feed point 211 of the first O2 flush supply line 11i. In the specific exemplary embodiment of FIG. 4, the bypass 75 can branch off, for example, between the inspiratory flow sensor 17 and the feed point 211 of the first O2 flush supply line 11i.

To suppress a backflow from the oxygen module 10 into the bypass 75 and/or the fan 3, at least one further check valve, namely a third safety valve 77, can advantageously be comprised. The third safety valve 77 can be arranged in the breathing gas line 4 between the junction of the bypass 75 and the feed point 211 of the first O2 flush supply line 11i. For example, the third safety valve 77 is designed as a simple check valve. It is also conceivable in some embodiments that the third safety valve 77 is designed as a spring-loaded check valve.

The bypass 75 can open back into the breathing gas line 4 between the patient interface 91 and the fan 3. The bypass 75 can therefore open into the expiratory branch 2. The bypass 75 can preferably open back into the breathing gas line 4 between the separating means 60 and the fan 3. The bypass 75 can in particular open back into the breathing gas line 4 between the separating means 60 and the anesthetic module 8. In the specific exemplary embodiment of FIG. 4, the bypass 75 can open, for example, after the second safety valve 22 into the expiratory branch 2 of the breathing gas line 4. The second safety valve 22 can then prevent the bypass flow S1 from running counter to the main flow S and in the direction of the separating means 60. The bypass 75 can branch off from the breathing gas line 4 after the fan 3 and open back into the breathing gas line 4 at least before the anesthetic module 8.

The bypass flow S1 can be generated by the bypass 75. The bypass flow S1 is not conducted to the patient 90 in the exemplary embodiment of FIG. 4. The bypass flow S1 is not generated at the opening of the oxygen module 10 in the exemplary embodiment of FIG. 4. The bypass flow S1 is not conducted through the separating means 60 in the exemplary embodiment of FIG. 4. The second safety valve 22 prevents a flow of the bypass flow S1 in the direction of the separating means 60.

The bypass flow S1 runs in this embodiment at least from the fan 3 through the bypass 75 to the anesthetic module 8 and again to the fan 3. A second gas circuit can therefore be formed by the bypass 75 with the bypass flow S1, which can exist alternatively or additionally to the gas circuit of the main flow S. Bypass flow S1 and main flow S can run parallel in the breathing gas line 4 at least in some areas. Bypass flow S1 and main flow S may not run in a common line at least in some areas.

In the case of liquid dosing, one or more anesthetics VA are kept under pressure and accordingly in liquid form in the anesthetic module 8 and/or the anesthetic supply line 9 and injected from there into the breathing gas line 4. Upon reaching the breathing gas line 4, the anesthetics VA vaporize and mix with the breathing gas mixture 5. Because a permanent gas flow exists due to the bypass flow S1 in the vaporization element 8a, the anesthetics VA can particularly advantageously admix with the breathing gas mixture 5.

Due to this type of the liquid dosing, prior mixing of anesthetics VA and fresh gas is no longer necessary. Therefore, a mixing chamber separate from the breathing gas line 4 for separate mixing of fresh gas or breathing gas and anesthetics VA is also no longer necessary. The liquid dosing enables very exact, frugal dosing of anesthetics VA, which is independent of the supply of fresh gas and/or oxygen. The vaporization element 8a can replace a classic mixing chamber.

FIG. 5 shows a schematic structure of the mechanical separating means 60 having diffusion filter 61. The mechanical separating means 60 can in principle be designed as a two-chamber system 65 and can comprise at least one first chamber 62 and at least one second chamber 63. The first chamber 62 and the second chamber 63 are separated from one another by the at least one diffusion filter 61.

The mechanical separating means 60 can comprise a large number of such two-chamber systems 65, which can extend in parallel to one another.

In one exemplary embodiment, the two-chamber systems 65 are designed as gas-carrying pipes. The pipes can have a diameter of 0.1 mm to 10 mm, preferably of 0.1 mm to 5 mm, particularly preferably of 0.3 to 1 mm. For example, the pipes of the two-chamber systems 65 have a diameter of 0.5 mm. For example, the mechanical separating means 60 comprise several thousand of such pipes (not shown). The total diameter of the pipes and therefore of the separating means 60 advantageously corresponds to the diameter of the breathing gas line 4.

The diffusion filter 61 can be designed as a semipermeable membrane and can have pores 65 which are configured such that only molecules which fall below a specific size can pass the membrane. The diffusion filter 61 is preferably formed from a chemically inert material. The diffusion filter 61 is distinguished in that it can be cleaned, disinfected, and reused.

For example, the diffusion filter 61 is produced from plastic, ceramic, glass, metal, or combinations thereof. In exemplary embodiments, the material of the diffusion filter 61 is selected from the group polysulfone, polyethersulfone, cellulose, cellulose ester, cellulose acetate, cellulose nitrate, regenerated cellulose, silicone, polyamide, polyamide imide, polyamide urea, polycarbonate, ceramic, stainless steel, silver, silicon, zeolite, aluminosilicate, polyacrylonitrile, polyethylene, polypropylene, polytetrafluoroethylene, polyvinylidene fluoride, polyvinyl chloride, polypiperazinamide. Combinations of these materials are also possible.

The breathing gas mixture 5 can be conducted in the direction of the main flow S through the first chamber 62 of the two-chamber system 65. Preferably, expiratory breathing gas 5ₑₓₛₚ is conducted through the separating means 60 in order to separate CO2. The expiratory breathing gas 5ₑₓₛₚ can be conducted through the first chamber 62. For this purpose, the separating means 60 is preferably arranged in the expiratory branch 2. It is particularly advantageous if the separating means 60 is arranged very close to the patient 90. In some embodiments, the separating means 60 can alternatively or additionally also be configured to separate nitrous oxide (N2O). The separation of N2O can take place equivalently to the separation of CO2 described herein.

A sweep gas 64 can be conducted through the second chamber 63 of the two-chamber system 65. The sweep gas 64 is configured to wash at least CO2 out of the breathing gas mixture 5. The sweep gas 64 is conducted by a sweep gas flow S2 through the second chamber 63, the direction of which is indicated in FIG. 5 by an arrow S2. The direction of the sweep gas flow S2 is preferably selected counter to the direction of the main flow S of the breathing gas mixture 5, which is indicated here by an arrow S.

The device 100 can comprise at least one sweep gas module 70 and the at least one sweep gas supply line 71. Sweep gas 64 can be introduced into the separating means 60 via the sweep gas supply line 71. In particular, sweep gas 64 can be introduced via the sweep gas supply line 71 into the second chamber 63 of the separating means 60. Sweep gas 64 can also be discharged from the second chamber 63 via the sweep gas supply line 71. The sweep gas supply line 71 is pneumatically connected to the second chamber 63 for this purpose. The sweep gas supply line 71 can supply and/or discharge sweep gas 64 to or from the separating means 60.

The sweep gas 64 can in some exemplary embodiments be acquired from pressurized gas cylinders. The sweep gas 64 can alternatively or additionally also be acquired via the oxygen module and/or the fresh gas module 6.

The control unit 101 and/or the sweep gas module 70 can specify the composition of the sweep gas 64. The control unit 101 and/or the sweep gas module 70 can specify a flow and/or volume of the sweep gas 64. The sweep gas module 70 can be connected to the control unit 101 and/or can be controlled by the control unit 101.

The control unit 101 and/or the sweep gas module 70 can also be configured to suppress or end the sweep gas flow so that the device 100 can also be operated without sweep gas 64. This is advantageous, for example, when the device 100 is operated in a semi-open circuit, for example, in the TIVA mode. Without a sweep gas flow in the second chamber 63, the separating means 60 is inactive. The breathing gas mixture 5 can then flow through the mechanical separating means 60 without gas components such as CO2 being separated.

The sweep gas 64 is configured such that the concentration of CO2 is significantly below that of the breathing gas mixture 5. The sweep gas 64 is configured such that the CO2 concentration of the sweep gas 64 upon introduction into the second chamber 63 is less than 20%, preferably less than 10%, particularly preferably less than 5%. Ideally, the CO2 concentration of the sweep gas 64 upon introduction into the second chamber 63 is 0%. The sweep gas 64 is moreover advantageously free of anesthetics VA.

The sweep gas 64 is moreover configured such that the concentration of oxygen O2 and/or nitrogen N2 can preferably be at least equal to or greater than that of the expiratory breathing gas mixture 5.

The O2 concentration of the sweep gas 64 upon introduction into the second chamber 63 is greater than 0%, preferably greater than 20%, particularly preferably greater than 40%. For example, the O2 concentration of the sweep gas 64 upon introduction into the second chamber 63 can be 47%.

The N2 concentration of the sweep gas 64 upon introduction into the second chamber 63 is greater than 0%, preferably greater than 20%, particularly preferably greater than 40%. For example, the N2 concentration of the sweep gas 64 upon introduction into the second chamber 63 can be 53%.

Due to a concentration gradient across the diffusion filter 61, the breathing gas mixture 5 can be separated in the mechanical separating means 60. Particularly small molecules can be separated due to the composition of the diffusion filter 61. In particular, molecules can be separated which consist of few atoms, for example fewer than 5 atoms. In particular, CO2 modules can be separated. Oxygen O2 and nitrogen N2 can also pass the diffusion filter 61. Anesthetics VA which have more than 5 atoms, in contrast, cannot pass the diffusion filter 61 and remain in the breathing gas mixture 5.

It is possible to control via the initial concentration of the gas components of the sweep gas 64 which gas components are filtered out of the breathing gas mixture 5 and which gas components remain in the breathing gas mixture 5.

The following scenario can be advantageous with respect to carbon dioxide CO2:

If a sweep gas 64 is introduced into the second chamber 63, which contains a lower CO2 concentration than the breathing gas mixture 5 in the first chamber 62, CO2 can be filtered out of the breathing gas mixture 5. CO2 can thus be withdrawn from the breathing gas mixture 5. The breathing gas mixture 5 can thus again be suitable for inspiration. The breathing gas mixture 5 can therefore be guided in a closed circuit.

The following scenarios can be advantageous with respect to oxygen O2 and/or N2:

If sweep gas 64 is introduced into the second chamber 63 which contains the same and/or a higher O2 concentration than the breathing gas mixture 5 in the first chamber 62, O2 can remain in the breathing gas mixture 5. If the breathing gas mixture 5 is then again used for an inspiration, it contains oxygen in the concentration of the sweep gas 64.

If sweep gas 64 is introduced into the second chamber 63 which contains a lower O2 concentration than the breathing gas mixture 5 in the first chamber 62, O2 can be filtered out of the breathing gas mixture 5. This can offer the advantage that oxygen which was temporarily provided in excess to the patient can be reused and/or that the breathing gas mixture 5 can be reused in spite of temporary O2 excess.

The remaining or the withdrawal of N2 and/or N2O in or from the breathing gas mixture 5 can also be regulated according to the same principle.

The gas concentrations of the introduced sweep gas 64 can be controlled via the control unit 101 and/or via the sweep gas module 70. The volume and/or the flow of the introduced sweep gas 64 can also be controlled via the control unit 101 and/or via the sweep gas module 70. In some exemplary embodiments, the sweep gas flow through the separating means 60 is constant. In some embodiments, the sweep gas flow can also be adjusted adaptively to the ventilation and therefore, for example, to the breath volume or the minute volume. The control is preferably carried out automatically by specifications and values stored in the control unit 101, but can also be set manually by trained medical personnel if needed.

In some embodiments, the sweep gas flow and/or the sweep gas volume corresponds to the flow and/or the volume of the breathing gas mixture 5. In advantageous exemplary embodiments, the sweep gas 64 is introduced into the separating means 60 in excess in relation to the breathing gas mixture 5. For example, just as much sweep gas 64 is introduced into the second chamber 63 per breath as breathing gas mixture 5 flows into the first chamber 62. Advantageously, more sweep gas 64 is introduced into the second chamber 63 per breath than breathing gas mixture 5 flows into the first chamber 62.

The ratio of the amount of sweep gas 64 to breathing gas mixture 5 can be at least 1:1, preferably at least 1.2:1, particularly preferably at least 1.4:1. For example, the ratio of sweep gas 64 to breathing gas mixture 5 is 1.5:1 or more. A ratio of sweep gas 64 to breathing gas mixture 5 of at least 2:1 or at least 5:1 is also conceivable. The sweep gas is typically dosed such that it is 1.2 to 1.5 times the minute volume.

The ratio of the amount of sweep gas 64 to the breathing gas mixture 5 can be variably settable and adaptively adjustable to the corresponding ventilation situation. Preferably, the sweep gas flow can be controllable on the basis of the CO2 concentration of the expiratory breathing gas 5ₑₓₛₚ. The mechanical separating means 60 having the sweep gas module 70 offers the advantage that the CO2 concentration of the breathing gas mixture 5 is settable quickly, in a defined manner, highly dynamically, and cost-effectively. Since a high CO2 concentration stimulates the patient to spontaneous breathing, the CO2 concentration can influence, for example, the weaning of the patient from the machine ventilation.

The sweep gas supply line 71 can be constructed corresponding to the supply lines according to FIG. 2. The sweep gas supply line 71 can also have more or fewer elements than the supply lines according to FIG. 2. The sweep gas supply line 71 can comprise at least one valve 88i for dosing sweep gas 64.

The sweep gas module 70 can in particular be supplied with gas by the fresh gas module 6 and/or the oxygen module 10. The sweep gas supply line 71 can then comprise at least one bistable switching valve 87 and/or at least one bistable needle valve 88 and at least one flow sensor 85, via which the sweep gas introduction into the separating means 60 is regulated in a supervised manner (not shown).

The advantages of the mechanical separating means 60 in relation to a chemical separating means 40 are as follows:
Substantial savings of anesthetics VA, since they are not filtered out but rather remain in the circuit and therefore in the breathing gas mixture 5
The mechanical separating means 60 is chemically neutral or inert
No heat development and no water formation takes place during CO2 separation
No chemical hazardous waste thus results
The mechanical separating means 60 is designed for very long use (up to a year) and rarely requires a replacement of the separating means 60
The separating means 60 can therefore be replaced by a service employee
There is a lower risk of contamination due to the rarer replacement
The separating means 60 can optionally be reused after processing
The separating means 60 is more cost-effective than chemical separating means 40 due to its long life

Embodiments having a mechanical separating means 60 and a bypass 75 offer many advantages. A permanent gas flow (byflow) can be provided at least in some sections in the breathing gas line 4 by the bypass 75. In particular, a permanent gas flow can be provided by the bypass 75 in the area of the breathing gas line 4 into which the anesthetic supply line 9 opens. In particular, a permanent gas flow can be applied in the vaporization element 8a due to the bypass 75. Because a permanent flow (byflow) of the breathing gas mixture 5 prevails in the vaporization element 8a, the anesthetics VA can particularly advantageously be introduced and/or mixed into the breathing gas mixture 5 via liquid dosing.

The advantage of exemplary embodiments having at least one bypass 75 is that the byflow is not conducted through the entire system. The byflow can be restricted to the second circuit K2. The bypass 75 enables the byflow not to flow to the patient. Moreover, the bypass 75 enables the byflow not to flow through the mechanical separating means 60. This offers the special advantage that the expiratory breathing gas 5ₑₓₛₚ is introduced in undiluted form into the separating means 60. This significantly increases the efficiency of the mechanical separating means 60. On the one hand, due to the absent byflow in the separating means 60, the CO2 concentration is high upon introduction into the separating means 60, which advantageously influences CO2 separation. On the other hand, the dwell time of the expiratory breathing gas 5ₑₓₛₚ in the separating means 60 increases. In the expiratory plateau, no gas flow takes place in the separating means 60, which promotes the diffusion of CO2 in the sweep gas.

The connection of the bypass conducting breathing gas can be established or blocked by the bypass shutoff valve 76. Blocking of the bypass 75 can be advantageous if the concentration of the anesthetics VA or of the oxygen in the breathing gas mixture 5 in the inspiratory branch 1 is to be changed rapidly. It has to be noted in this case that in this time the efficiency of the diffusion absorber is reduced.

The device 100 of the second exemplary embodiment can be operated in the different operating modes M1, M2, M3, M4, M5, M6, M7 described for FIGS. 1A-1F. The operating modes can be set manually and/or specified automatically by the control unit 101.

Volatile anesthetics VA can also be applied, conducted, and in particular also deliberately discharged for disposal or for reuse using the device 100 in the second exemplary embodiment. In the second exemplary embodiment, ventilation without volatile anesthetics VA can also be carried out using the device 100.

The operating modes in the second exemplary embodiment can be implemented, for example, via the settings of the valves 25, 26, 27, 28, 29 and optionally also via the settings of the fan 3 and/or the pressure relief valve 30 and via the operation of the reservoir 12. The operating modes in the second exemplary embodiment can in particular also be influenced via the setting of the bypass shutoff valve 76.

The operating modes M1 to M7 of the second exemplary embodiment correspond in large parts to the operating modes of the first exemplary embodiment listed above. However, there are differences in particular due to the bypass and the differing separating means 40, 60, which are described hereinafter.

In the first operating mode M1 for manual ventilation with application of volatile anesthetics, the reservoir 12 also supplies the conveying energy for the breathing gas mixture 5 and therefore the breathing energy in the second exemplary embodiment. The inspiratory breathing gas 5ᵢₙₛₚ is conducted to the patient via the inspiratory branch 1 and the expiratory breathing gas 5ₑₓₛₚ is conducted away from the patient via the expiratory branch 2. Fresh gas switching valve 32 and O2 flush switching valve 31 are in their base positions. The fresh gas introduction can therefore take place in the flow direction after the check valve 21. The optional oxygen introduction via the O2 flush 10 can take place in the flow direction after the fan 3 and before the safety valves 23, 24. The anesthetic module 8 is active so that volatile anesthetics VA can be introduced via the anesthetic supply line 9 into the vaporization element 8a and added to the breathing gas mixture 5.

The expiratory breathing gas 5ₑₓₛₚ can be conducted away from the patient via the expiratory branch 2. In the expiratory branch 2, the expiratory breathing gas 5ₑₓₛₚ passes the mechanical separating means 60. The sweep gas module 70 is active in the first operating mode M1 and conducts sweep gas in the separating means 60. The separating means 60 is thus also active and can separate at least CO2 from the expiratory breathing gas 5ₑₓₛₚ. The expiratory breathing gas 5ₑₓₛₚ can then pass the active PEEP valve 30, which regulates to an individual exhalation pressure.

The breathing gas mixture 5 can then also take two different paths in the second exemplary embodiment:

The breathing gas mixture 5 can, on the one hand, be conducted in the direction of the main flow S in the circuit, since the shutoff valve 28 is subjected to a low operating pressure and releases the breathing gas line 4. In contrast to the first exemplary embodiment, the breathing gas mixture 5 does not have to pass a chemical CO2 absorber 40 and therefore also does not have to pass a moisture control module 41 in this case. Since the CO2 has already been separated by the mechanical separating means 60, the breathing gas mixture 5 can be guided in the circuit and supplied to the patient again. However, it is also conceivable that in the second exemplary embodiment, a moisture control module 41 is comprised in order to withdraw and/or supply moisture from or to the circuit (not shown).

On the other hand, the breathing gas mixture 5 can also be discharged at least in parts via the APL valve 29 through the conduction system 14 from the circuit.

In contrast to the first exemplary embodiment according to FIG. 1, in the second exemplary embodiment according to FIG. 4, a second circuit having a bypass flow S1 can exist. The bypass 75 is configured and designed to permit the second circuit if needed.

In the first operating mode M1, the bypass shutoff valve 76 is subjected to a low operating pressure or is in an idle position. The bypass shutoff valve 76 can act as a simple check valve in the first operating mode M1 without action of the solenoid. In the first operating mode M1, the bypass shutoff valve 76 releases the bypass 75 in the direction of the bypass flow S1.

The bypass 75 offers the advantage that a second circuit is created for the breathing gas mixture 5, which is not conducted to the patient. A nearly constant flow can be generated in the second circuit which is independent of the breathing phases of the patient. This nearly constant flow then also runs through the vaporization element 8a and promotes the liquid dosing of the anesthetics. In addition, the bypass 75 offers the advantage due to the creation of the second circuit that a lesser flow is conducted through the separating means 60 and therefore sweep gas 64 can be saved.

In the second operating mode M2 for machine ventilation with application of volatile anesthetics, the fan 3 also supplies the conveying energy for the breathing gas mixture 5 and therefore the breathing energy in the second exemplary embodiment. The reservoir 12 is used as a store for at least a part of the breathing gas mixture 5. The fresh gas switching valve 32 and the O2 flush switching valve 31 are in the base position. Fresh gas is introduced via the first fresh gas supply line 7i into the breathing gas line 4 and conveyed by the fan 3 to the patient. The anesthetic module 8 is active. In the second exemplary embodiment, the sweep gas module 70 and therefore also the separating means 60 are also active in the operating mode M2, so that at least CO2 can be separated and the breathing gas mixture 5 can at least partially remain in the circuit.

During operation of the fan 3, the fan 3 can aspirate the breathing gas mixture 5 (enriched with fresh gas and/or anesthetics) from the breathing gas line 4, so that a negative pressure can at least partially arise upstream before the fan 3. Breathing gas mixture 5 can thus be aspirated from the reservoir 12. If the negative pressure remains, for example, if the fresh gas module 6 and/or the reservoir 12 provides too little volume, the inlet valve 27 can open and supply ambient air to the breathing gas line 4.

The feed point for ambient air 227 can be arranged in the breathing gas line 4. The feed point for ambient air 227 can also be arranged in the reservoir line 13 in some exemplary embodiments. As an example, the feed point for ambient air 227 can be located in the reservoir line 13 in the second exemplary embodiment of FIG. 4. In this specific exemplary embodiment, the feed point for ambient air 227 can be located between the reservoir 12 and the reservoir feed point 213 in the breathing gas line 4. Ambient air can thus be fed via the reservoir line 14 directly into the breathing gas line 4.

The valves 26, 28, 29 are switched in the second operating mode M2 such that the breathing gas mixture 5 runs essentially in the circuit along the main flow direction S, in contrast to the first exemplary embodiment, however, the breathing gas mixture 5 in the second exemplary embodiment does not pass a chemical separating means 40. A moisture control module 41 can optionally be included, but can be superfluous due to the absent chemical absorber 40, however.

The bypass shutoff valve 76 of the second exemplary embodiment is in its idle position in the second operating mode M2. The bypass 75 is therefore active and can have flow through it in the direction of the bypass flow S1. In the operating mode M2 of the second exemplary embodiment, there is therefore a second circuit for the breathing gas mixture 5 as in the first operating mode M1.

The third operating mode M3 for manual ventilation without application of volatile anesthetics can be very similar in the first and in the second exemplary embodiment. The breathing gas mixture 5 can be guided identically or similarly, for example. This can be achieved in particular in that in the third operating mode M3, the bypass 75 and/or the sweep gas module 70 and/or the mechanical separating means 60 of the second exemplary embodiment are inactive.

In the third operating mode M3, the bypass shutoff valve 76 can be subjected to a high operating pressure and/or can be in an active position. The bypass shutoff valve 76 can act as a shutoff valve under the action of the solenoid in the third operating mode M3. In the third operating mode M3, the bypass shutoff valve 76 can block the bypass 75 such that the bypass 75 is inactive. In the third operating mode M3, the second circuit for the breathing gas mixture 5 is prevented by the active bypass shutoff valve 76.

In the third operating mode M3, the sweep gas module 70 can be inactive. An inactive sweep gas module 70 does not convey sweep gas 64 in the mechanical separating means 60. Because no sweep gas flow is applied in the separating means 60, the mechanical separating means 60 is inactive. Passive flow can occur through the separating means 60. The expiratory breathing gas mixture 5ₑₓₛₚ can flow through the separating means 60 without CO2 being separated in the third operating mode M3.

Since in general no volatile anesthetics VA are added to the breathing gas mixture 5 in the third operating mode M3, the expiratory breathing gas 5ₑₓₛₚ can be guided in a semi-open circuit as described above by blocking the shutoff valve 28 and opening the discharge valve 26.

The fourth operating mode M4 for automatic ventilation without application of volatile anesthetics can also be very similar in the first and in the second exemplary embodiment. The breathing gas mixture 5 can be guided identically or similarly, for example. This can be achieved in particular in that the bypass 75 and/or the sweep gas module 70 and/or the mechanical separating means 60 of the second exemplary embodiment are also inactive in the fourth operating mode M4. As in the first exemplary embodiment, the fan 3 is active and the overflow valve 25 is also subjected to a low operating pressure to activate the first line 14i in the second exemplary embodiment to implement the fourth operating mode M4. Moreover, the shutoff valve 28 is active to prevent the circuit and the discharge valve 26 is open to activate the third line 14iii. In contrast to the first exemplary embodiment, in the second exemplary embodiment the ambient air can be fed if needed directly into the reservoir line 13 through the inlet valve 27.

The device 100 according to the second exemplary embodiment can be operated for manual ventilation in a battery and/or error mode in the fifth operating mode M5. The fifth operating mode M5 can be similar in the first and in the second exemplary embodiment. An emergency supply via manual ventilation can be ensured in that the reservoir 12 supplies the conveying energy for the breathing gas mixture 5 and fresh gas can be fed with a constant flow via the first fresh gas supply line 7i into the breathing gas line 4. The fresh gas supply line 7i is open when deenergized.

The flow path of the breathing gas fundamentally runs as in the first exemplary embodiment from the reservoir 12 via the inspiratory branch 1 to the patient and from the patient via the expiratory branch 2 in the direction of the main flow S in the circuit and/or via the APL valve 29 and the second line 14ii to the outlet 14-A.

In contrast to the first exemplary embodiment, the bypass 75 can be active in the second exemplary embodiment. The bypass shutoff valve 76 can act as a simple check valve when deenergized. The breathing gas mixture 5 can then additionally be guided with a relatively constant flow in the second circuit.

In the fifth operating mode M5, the sweep gas module 70 is active when deenergized, so that sweep gas 64 can be introduced into the mechanical separating means 60 and CO2 can be separated. The sweep gas supply line is open when deenergized. The valve 88i for dosing sweep gas 64 can be configured to set the sweep gas dosing when deenergized to 1.2 to 2 times the last set minute volume, preferably to 1.5 times. The advantage of the second exemplary embodiment is that no particular moisture development arises due to the mechanical CO2 separation. Moreover, the CO2 separation is secured by the sweep gas module 70 which is active when deenergized.

The sixth operating mode M6 for a constant flow (HFOT), with or without application of volatile anesthetics, can be very similar in the first and in the second exemplary embodiment. The breathing gas mixture 5 can be guided identically or similarly, for example. This can be achieved in particular in that in the sixth operating mode M6, the bypass 75 of the second exemplary embodiment is inactive.

The bypass shutoff valve 76 can be subjected to a high operating pressure and/or can be in an active position in the sixth operating mode M6. The bypass shutoff valve 76 can therefore inactivate or block the bypass 75. An inactive bypass 75 in the sixth operating mode M6 means that the breathing gas mixture 5 cannot pass the bypass 75. The breathing gas mixture 5 is then conducted as described above in an open circuit. The breathing gas mixture 5 is conducted via the inspiratory branch 1 to the patient, so that the patient can be supplied with a constant flow.

The seventh operating mode M7 can be implemented similarly in the second exemplary embodiment to the first exemplary embodiment. The device 100 according to the second exemplary embodiment can therefore also be operated in a service mode. The seventh operating mode M7 is configured and designed in particular to remove condensed moisture from the device 100. Although in principle less moisture can arise in the device 100 due to the advantageous mode of operation of the mechanical separating means 60 in relation to the chemical separating means 40, it is possible to implement the seventh operating mode M7 in order to remove condensed moisture.

In the seventh operating mode M7, with operation of the fan 3 and an inactive fresh gas module 6, ambient air can be conveyed via the inlet valve 27 into the reservoir line 13 and from there into the breathing gas line 4. The gases can be discharged again via the outlet 14-A. In the seventh operating mode M7, the bypass 75 can be active and can have flow through it. It is also conceivable that the bypass 75 is inactivated by activating the bypass shutoff valve 76. In operation in the seventh operating mode M7, the sweep gas module 70 can be inactive and the mechanical separating means 60 can have passive flow through it.

Although the present invention was described in detail on the basis of the exemplary embodiments, it is self-evident for a person skilled in the art that the invention is not restricted to these exemplary embodiments. Rather, modifications are possible in such a manner that individual features are omitted or different combinations of the described individual features can be implemented, provided that the scope of protection of the appended claims is not departed from. The present invention includes all combinations of the presented individual features.

### LIST OF REFERENCE SIGNS

CO2 carbon dioxide
O2 oxygen
K1 first circuit
K2 second circuit
M1 first operating mode
M2 second operating mode
M3 third operating mode
M4 fourth operating mode
M5 fifth operating mode
M6 sixth operating mode
M7 seventh operating mode
N2 nitrogen
P1 first pressure
P2 second pressure
Pᵢₙₛₚ inspiration pressure
Pₑₓₛₚ expiration pressure
PEEP positive end-expiratory pressure
S main flow
S1 bypass flow
S2 sweep gas flow
S3 outlet flow
VA volatile anesthetics
CGS central gas system
1 inspiratory branch
2 expiratory branch
3 fan unit/fan
3a fan outlet
4 breathing gas line
5 breathing gas mixture
5ᵢₙₛₚ inspiratory breathing gas
5ₑₓₛₚ expiratory breathing gas
6 fresh gas module
7 fresh gas supply line
7i first fresh gas supply line
7ii second fresh gas supply line
8 anesthetic module
8a vaporization element
9 anesthetic supply line
10 oxygen module/O2 flush
11 O2 flush supply line
11i first O2 flush supply line
11ii second O2 flush supply line
12 reservoir
13 reservoir line
14 conduction system
14i first line
14ii second line
14iii third line
14-A outlet
15 inspiratory pressure sensor
16 expiratory pressure sensor
17 inspiratory flow sensor
18 expiratory flow sensor
19 sensor
20 multigas sensor
21 first check valve
22 second check valve
23 first safety valve
24 second safety valve
25 overflow valve
26 discharge valve
27 inlet valve
28 shutoff valve
29 APL valve
30 pressure control valve (PEEP valve)
31 O2 flush switching valve
32 fresh gas switching valve
35 manual bag
39 pressure sensor
40 chemical separating means
41 moisture control module
43 nitrogen oxide module
44 nitrogen oxide supply line
60 mechanical separating means
61 diffusion filter
62 first chamber
63 second chamber
64 sweep gas
65 two-chamber system
70 sweep gas module
71 sweep gas supply line
75 bypass
76 bypass shutoff valve
77 third safety valve
80 pressure regulator/pressure reducer
81 stenosis
82 filter
83 overpressure valve
84 pressure sensor
85 flow sensor
86 check valve
87 switching valve
88 dosing valve
89 supply source
90 patient
91 patient interface
92 tubing system
93 fitting for patient interface
94 filter
95 sensor
100 device
101 control unit
102 storage unit
103 primary power source
104 secondary power source
110 ventilation-specific parameters
111 patient parameters
207 fresh gas feed point
209 anesthetic feed
211 O2 flush feed point
213 reservoir feed point
214 connection point (conduction system)
227 ambient air feed point
800 tank
810 pressure supply unit
811 valve for loading
812 valve for relief
813 switching valve
820 receptacle unit
821 receptacle bay
830 selection unit
831 selection valve
840 dosing unit
841 dosing valve
850 safety unit
851 first valve (safety valve)
852 second valve (safety valve)
860 temperature unit
861 heating element

1.-55. (canceled)

56. A device for supplying breathing gas, wherein the device comprises at least one breathing gas line for conducting a breathing gas mixture, which breathing gas line comprises at least one outlet, via which the breathing gas mixture can be at least partially, at least temporarily discharged, an inspiratory branch, which is configured to conduct breathing gas to a fitting for a patient interface, an expiratory branch, which is configured to conduct breathing gas between the fitting for a patient interface and the outlet, at least one shutoff valve which is configured to at least temporarily establish a breathing gas conducting connection from the expiratory branch to the inspiratory branch being arranged in the breathing gas line.

57. The device of claim 56, wherein the shutoff valve is designed as a check valve and is configured to establish the breathing gas conducting connection in a flow direction from the expiratory branch to the inspiratory branch.

58. The device of claim 56, wherein the shutoff valve is designed as a blockable check valve and is configured to at least temporarily block the breathing gas line in both flow directions.

59. The device of claim 56, wherein the device further comprises a control unit and at least one power source, and wherein the shutoff valve is configured to block the breathing gas line in both flow directions when energized by the at least one power source.

60. The device of claim 56, wherein the shutoff valve is configured, when energized, to suppress the breathing gas conducting connection from the expiratory branch to the inspiratory branch and/or wherein the breathing gas mixture can be completely discharged via the outlet from the expiratory branch when the shutoff valve is energized.

61. The device of claim 56, wherein inspiratory branch and expiratory branch, when the shutoff valve is not energized, form at least one first circuit (K1), in which the breathing gas mixture can be conducted, the breathing gas mixture being be at least partially dischargeable via the outlet.

62. The device of claim 56, wherein the device further comprises at least one controllable pressure control valve, which is configured to control an inspiration pressure (Pinsp) and/or an expiration pressure (Pexsp), the pressure control valve being manually and/or electrically settable.

63. The device of claim 56, wherein the pressure control valve is designed as an APL valve for controlling the inspiration pressure (Pinsp), wherein the breathing gas mixture can be discharged via the outlet if the pressure in the breathing gas line exceeds the inspiratory pressure (Pinsp), the APL valve being configured as a variably loaded check valve, and/or comprises a stepping motor, via which the APL valve is settable, the valve position of the APL valve being set and/or maintained while energized and/or remains at the last set value without energizing.

64. The device of claim 63, wherein the pressure control valve is designed as a valve for controlling the expiration pressure (Pexsp), and is arranged in the expiratory branch and configured in particular to control an end-expiratory expiration pressure (PEEP), and is configured to regulate passively to a preset expiration pressure (Pexsp) without being energized, the preset expiration pressure (Pexsp) of the pressure control valve being 3 hPa to 10 hPa.

65. The device of claim 56, wherein the device further comprises at least one anesthetic supply line for introducing volatile anesthetics (VA) into the breathing gas line, the breathing gas line being configured such that, in operation of the device, a first pressure (P1) is applied in the breathing gas line, and the anesthetic supply line being configures such that, in operation of the device, a second pressure (P2) is applied in the anesthetic supply line, the first pressure (P1) being lower than the second pressure (P2).

66. The device of claim 65, wherein the anesthetic supply line is configured to conduct the volatile anesthetics (VA) in liquid form to the breathing gas line, the volatile anesthetics (VA) vaporizing upon introduction into the breathing gas line at a vaporization rate of 0 to 2 l/min.

67. The device of claim 56, wherein the device comprises at least one safety valve, which is configured and designed to block a supply of volatile anesthetics (VA), the safety valve being configured as a switching valve, which is electrically and/or manually settable without and is configured without energizing to block the introduction of volatile anesthetics (VA) into the breathing gas mixture.

68. The device of claim 56, wherein the outlet comprises at least one filter, which is placed such that breathing gas mixture discharged via the outlet completely passes the filter, the filter being or comprising an absorbent and being configured and designed to absorb at least volatile anesthetics (VA) and/or metabolites thereof, a double filter system having a first filter and a second filter being arranged at the outlet and the second filter being arranged after the first filter in flow direction.

69. The device of claim 56, wherein the device further comprises at least one sensor, which is arranged in or at the outlet and is designed and configured to detect a concentration of volatile anesthetics (VA) and/or metabolites thereof in flow direction at least before and/or after the first filter and transmit same to the control unit, the control unit being configured to generate an alarm if the concentration of volatile anesthetics (VA) and/or metabolites thereof exceeds a limiting value.

70. The device of claim 56, wherein the device further comprises a separating element, which is configured to separate at least CO2 from the breathing gas mixture and is a chemical separating element and/or a mechanical separating element, the mechanical separating element being arranged in the expiratory branch and comprising at least one diffusion filter, which is designed as a semipermeable membrane and is permeable at least to CO2 molecules and/or is not permeable at least to volatile anesthetics (VA).

71. The device of claim 70, wherein the separating element is designed as a two-chamber system, which comprises at least one first chamber and at least one second chamber, the first chamber and the second chamber being gas-conducting and being separated from one another by the diffusion filter, and/or the first chamber being configured to accommodate expiratory breathing gas (5ₑₓₛₚ) and the second chamber being configured to accommodate a sweep gas, wherein the sweep gas at least has a lower CO2 concentration than expiratory breathing gas (5ₑₓₛₚ).

72. The device of claim 70, wherein the first chamber is configured to guide expiratory breathing gas (5ₑₓₛₚ) in a direction (S) of a main flow, and the second chamber is configured to guide the sweep gas (S2) in a direction of a sweep gas flow, when the flow direction (S) of the main flow is opposite to the flow direction (S2) of the sweep gas flow.

73. The device of claim 56, wherein the device further comprises at least one sweep gas supply line for providing sweep gas and at least one valve for dosing sweep gas for the mechanical separating element and/or further comprises at least one valve for dosing fresh gas and/or oxygen O2, and/or the device further comprises at least one valve for dosing volatile anesthetics (VA), the dosing valves being configured as needle valves each having at least one stepping motor, via which the dosing valves are settable, a valve position of the dosing valves being set and/or maintained while energized, and without being energized remains at the last set value and/or falls to an open base position.

74. The device of claim 56, wherein the breathing gas line comprises a bypass, which branches off from the inspiratory branch and opens between a mechanical separating element and the outlet into the expiratory branch, the bypass being configured to at least temporarily establish a breathing gas conducting connection from the inspiratory branch to the expiratory branch such that a second circuit (K2) is formed, in which the breathing gas mixture can be conducted.

75. The device of claim 74, wherein the second circuit (K2) at least partially corresponds to the first circuit (K1), the fitting for the patient interface being only arranged in the first circuit (K1) and/or the mechanical separating element being only arranged in the first circuit (K1).

76. The device of claim 74, wherein the bypass comprises at least one bypass shutoff valve, which is designed and configured to at least temporarily establish the conducting breathing gas connection in a flow direction from the inspiratory branch to the expiratory branch.

77. The device of claim 76, wherein the bypass shutoff valve is designed and configured as a blockable check valve, to at least temporarily block the bypass in both flow directions, the bypass shutoff valve being configured, when energized by at least one power source, to block the bypass in both flow directions and/or the breathing gas mixture being be at least partially conductible in the second circuit (K2) without energizing of the shutoff valve and the bypass shutoff valve.

78. The device of claim 56, wherein the device is configured to conduct the breathing gas mixture in the first circuit (K1) with a main flow (S) and to conduct it in the second circuit (K2) with a bypass flow (S1), the main flow (S) being dependent on breathing phases of a patient to be ventilated and/or at least the bypass flow (S1) being independent of the breathing phases of a patient to be ventilated and/or the bypass flow (S1) of the second circuit (K2) being conducted through a PEEP valve.
